(19) United States
(12) Patent Application Publication
(10) Pub. No.: US 2026/0250685 A1
(43) Pub. Date: Aug. 27, 2026

(54) ANTISENSE OLIGONUCLEOTIDE AGENTS FOR TREATING CONDITIONS ASSOCIATED WITH NLRP3/NLRP1 EXPRESSION OR ACTIVATION

(71) Applicant: Molecular Axiom, LLC, San Diego, CA (US)

(72) Inventors: Lishan CHEN, San Diego, CA (US); Bohan JIN, San Diego, CA (US); Timothy WONG, San Diego, CA (US)

(21) Appl. No.: 19/163,099

(22) PCT Filed: Mar. 1, 2024
(86) PCT No.: PCT/US2024/018008

§ 371 Date: Sep. 8, 2025

Related U.S. Application Data
(60) Provisional application No. 63/449,178, filed on Mar. 1, 2023.

(51) Int. Cl. C12N 15/113

(52) U.S. Cl. CPC C12N 15/1137; C12N 2310/11; C12N 2310/313; C12N 2310/315; C12N 2310/321; C12N 2310/322; C12N 2310/3231

(57) ABSTRACT

The present disclosure provides antisense oligonucleotide compounds and compositions (e.g., pharmaceutical compositions) targeting NLRP3 and/or NLRP1 mRNA, and methods for treating diseases or conditions associated with NLRP3 and/or NLRP1 mRNA expression or NLRP3 and/or NLRP1 inflammasome activation. Exemplary conditions for which the compounds and compositions find use include inflammatory diseases such as neurodegenerative and autoimmune diseases, among others.

### PRIORITY

This Application claims the benefit of, and priority to, U.S. provisional application No. 63/449,178 filed Mar. 1, 2023, which is hereby incorporated by reference in its entirety.

### SEQUENCE LISTING

The instant application contains a sequence listing, which has been submitted in XML format via EFS-Web. The contents of the XML copy named “MAX-003PC_133279-5003 Sequence Listing”, which was created on Feb. 29, 2024, is 158,135 bytes in size, the contents of which are hereby incorporated by reference in their entirety.

### BACKGROUND

Inflammasomes are innate immune system receptors that detect pathogenic endo- and exogenous stressors and which activate the highly proinflammatory cytokines interleukin-1β and interleukin-18. Inflammasomes are not only involved in inflammation, but also in carcinogenesis and tumor progression

NLRP3 and NLRP1 are proteins that that are encoded by the NLRP3 and NLRP1 genes. NLRP3 and NLRP1 are components of the innate immune system that function as pattern recognition receptors (PRR) that recognize pathogen-associated molecular patterns (PAMPs). Danger signals are a hallmark of many inflammatory diseases, and these stimuli can function to activate NLRP3 or NLRP1. Once activated, NLRP3 or NLRP1 nucleates the assembly of an inflammasome, leading to caspase 1-mediated proteolytic activation of the interleukin 1φ (IL 1β) family of cytokines, which induces inflammation and can induce cell death. The activation of NLRP3 or NLRP1 contributes to various inflammatory diseases, including several neurodegenerative, inflammatory, and autoimmune conditions. Thus, there is a need for therapies to inhibit or reduce aberrant or persistent activation of the NLRP3 or NLRP1 inflammasome.

### DETAILED DESCRIPTION

The present disclosure provides antisense oligonucleotide compounds and compositions (e.g., pharmaceutical compositions) targeting NLRP3 and/or NLRP1 mRNA, and methods for treating diseases or conditions associated with NLRP3 and/or NLRP1 mRNA expression or associated with persistent or aberrant activation of NLRP3 and/or NLRP1. Exemplary conditions for which the compounds and compositions find use include neurodegenerative, inflammatory, and autoimmune diseases.

In one aspect, the disclosure provides an antisense oligonucleotide of 10 to 30 nucleotides in length, and comprising at least 8 contiguous nucleotides of any one of SEQ ID NOs:1 to SEQ ID NO:52, and which reduces the expression of a NOD-, LRR-, pyrin domain-containing protein 1 (NLRP1) mRNA and/or pyrin domain-containing protein 3 (NLRP3) mRNA. In embodiments, the oligonucleotide reduces expression of both NLRP3 and NLRP1 mRNA. In various embodiments, the antisense oligonucleotide is complementary to an equal length portion of human NLRP3 mRNA. In various embodiments, the antisense oligonucleotide is complementary (or also complementary) to an equal length portion of a human NLRP1 mRNA. Exemplary NLRP3 and NLRP1 transcripts are disclosed in PCT/US2022/042394, which is hereby incorporated by reference in its entirety. NLRP3 and NLRP1 transcripts are further provided herein as SEQ ID NOs: 53-65. In embodiments, the antisense oligonucleotide targets (e.g., is complementary to an equal length portion of) a transcript of SEQ ID NO: 53. In embodiments, the antisense oligonucleotide targets (e.g., is complementary to an equal length portion of) a transcript of SEQ ID NO: 60.

In embodiments, the oligonucleotide is at least 12 nucleotides in length or is at least 14 nucleotides in length. In some embodiments, the oligonucleotide is from 10 to 24 nucleotides in length, such as 12, 13, 14, 15, 16, 17, 18, 19 or 20 nucleotides in length. In some embodiments, including embodiments illustrated in Tables 1 and 2, the oligonucleotide is 14 nucleotides in length. Tables 4 and 5 illustrate embodiments where the antisense oligonucleotide is from 14 to 20 nucleotides in length.

In various embodiments, the oligonucleotide comprises at least 10 or at least 12 contiguous nucleobases of any one of SEQ ID NOs: 1 to 52, and the oligonucleotide is substantially (e.g., at least 90%) or entirely complementary to a segment (e.g., an equal length segment) of an NLRP3 mRNA (human) (e.g., an mRNA of SEQ ID NOS: 53-59). In various embodiments, the oligonucleotide comprises at least 10 or at least 12 contiguous nucleobases of any one of SEQ ID NOs: 1 to 52, and the oligonucleotide is substantially (e.g., at least 90%) or entirely complementary to a segment (e.g., an equal length segment of an NLRP1 mRNA (human) (e.g., an mRNA of SEQ ID NOS: 60-65). In embodiments, the oligonucleotide is substantially (e.g., at least 90%) complementary or entirely complementary to a segment (e.g., an equal length segment) of an NLRP3 mRNA and an NLRP1 mRNA. As used herein, the term “complementary” refers to Watson-Crick base pairing, with the proviso that up to four bases in the oligonucleotide can be replaced with Inosine (e.g., deoxyInosine). In certain embodiments, the oligonucleotide has a nucleobase sequence of any one of SEQ ID NOs: 1 to 52. For simplicity nucleobase sequences may be shown herein using DNA nucleotide sequences (i.e., including T nucleobases). It is understood from the context that when a nucleotide is intended to be RNA (such as in an mRNA transcript), T nucleobases are substituted with U nucleobases. In the context of oligonucleotides, T and U nucleobases can be interchangeable. In embodiments, one or more nucleobases designated as T in an oligonucleotide, are U nucleobases.

In some embodiments, the oligonucleotide has a nucleobase sequence selected from Table 1 or Table 4. For example, the oligonucleotide may have a nucleobase sequence selected from SEQ ID NO: 1 to SEQ ID NO: 52. Various antisense oligonucleotides based on these sequences (as shown in Table 2) exhibited at least 40%, at least 50%, or at least 60% NLRP3 knockdown efficiency in THP-1 cells after 24 hours post-transfection at a concentration of 40 nM. Other antisense oligonucleotides based on these sequences (as shown in Table 5) exhibited at least 40%, at least 50%, or at least 60% NLRP3 and/or NLRP1 knockdown efficiency in U87-MG cells after 24 hours post-transfection at a concentration of 25 nM. Various antisense oligonucleotides based on these sequences (as shown in Table 5) exhibited at least 40%, at least 50%, or at least 60% NLRP3 and NLRP1 knockdown efficiency in U87-MG cells after 24 hours post-transfection at a concentration of 25 nM. These oligonucleotides include STN-101016, STN-101017, and STN-101020. Such oligonucleotides and other oligonucleotides based on these nucleobase sequences may be selected according to the present disclosure.

Additional oligonucleotide sequences and antisense oligonucleotides, which may be used in accordance with this disclosure, are disclosed in PCT/US2022/042394, which is hereby incorporated by reference in its entirety.

In some embodiments, the binding (e.g., hybridization) of the antisense oligonucleotide to the target mRNA leads to degradation of the target mRNA or blocks translation of the target mRNA. In some embodiments, the binding of the antisense oligonucleotide to the target mRNA creates a duplex nucleic acid molecule, which then recruits an endogenous nuclease for degradation of the mRNA. In some embodiments, the antisense oligonucleotide has a stretch of DNA nucleotides sufficient to recruit RNaseH, and thereby trigger degradation of the target mRNA. For example, the antisense oligonucleotide may have a stretch (e.g., a central stretch) of at least 6 or at least 8 DNA nucleotides, and which is optionally a stretch of 9 or 10 DNA nucleotides. In some embodiments, one or more DNA nucleotides comprise a 2′ chemical modification independently selected from 2′-Fluoro, 2′-Methyl, and 2′-Ethyl. In embodiments, the DNA nucleotides in the antisense oligonucleotide do not have any 2′ modification, meaning that they have a 2′-H. For example, the antisense oligonucleotide may be a gapmer having a 5′ and a 3′ segment, each of the 5′ and 3′ segments being from 2 to 6 nucleotides or from 2 to 4 nucleotides, and where the 5′ and 3′ segments do not contain DNA nucleotides. In some embodiments, the gapmer is a 3-8-3 gapmer, having a central block of 8 DNA nucleotides and 5′ and 3′ segments of 3 RNA nucleotides each. In other embodiments, the gapmer is a 2-10-2 gapmer, having a central block of 10 DNA nucleotides and 5′ and 3′ segments of 2 RNA nucleotides each. In embodiments, the gapmer is a 3-8-2 gapmer, 3-9-2 gapmer, 2-8-3 gapmer, or 2-9-3 gapmer. In embodiments, the gapmer is a 3-10-3 gapmer, 3-10-2 gapmer, 2-10-3 gapmer, 3-12-3 gapmer, 2-12-3 gapmer, 3-12-2 gapmer, 2-15-3 gapmer, 3-15-2 gapmer, 3-11-3 gapmer, 3-11-2 gapmer, 2-11-3 gapmer, 3-13-3 gapmer, 2-13-3 gapmer, or 3-13-2 gapmer. In some embodiments, one or more nucleotides of the 5′ segment and the 3′ segment comprise 2′-O substituents, optionally where all of the nucleotides of the 5′ segment and the 3′ segment comprise 2′-O substituents. Exemplary 2′-O substituents are independently selected from 2′-0 methyl, 2′-0 ethyl, 2′-0 methoxyethyl (MOE), and a bridged nucleotide (e.g., a locked or bi-cyclic nucleotide) having a 2′ to 4′ bridge. In some embodiments, the bridged nucleotide has a methylene bridge (locked nucleic acid, or LNA) or a constrained ethyl bridge (cEt).

The term “gapmer” refers to an oligonucleotide having a central block of deoxynucleotides (also referred to herein as “DNA nucleotides”) with 5′ and 3′ segments (of at least 2 nucleotides) of RNA nucleotides. As used herein, the term “DNA nucleotide” refers to a nucleotide that is not an RNA nucleotide. DNA nucleotides typically have a 2′ H, but may alternatively have various 2′ chemical modifications, including 2′-halo and 2′-lower alkyl (e.g., C1-4). In some embodiments, the 2′ chemical modifications of DNA nucleotides are independently selected from 2′-Fluoro, 2′-Methyl, and 2′-Ethyl. An “RNA nucleotide” will contain a 2′ hydroxyl, or a derivative of the 2′ hydroxyl (i.e., a 2′-O substituent).

Locked nucleic acid (LNA) or “locked nucleotides” are described, for example, in U.S. Pat. Nos. 6,268,490; 6,316,198; 6,403,566; 6,770,748; 6,998,484; 6,670,461; and 7,034,133, all of which are hereby incorporated by reference in their entireties. LNAs are modified nucleotides that contain a bridge between the 2′ and 4′ carbons of the sugar moiety resulting in a “locked” conformation, and/or bicyclic structure. Other suitable locked nucleotides that can be incorporated in the oligonucleotides of this disclosure include those described in U.S. Pat. Nos. 6,403,566 and 6,833,361, both of which are hereby incorporated by reference in their entireties. In exemplary embodiments, the locked nucleotides are independently selected from a 2′ to 4′ methylene bridge and a constrained ethyl (cEt) bridge (see, U.S. Pat. Nos. 7,399,845 and 7,569,686, which are hereby incorporated by reference in their entireties).

In some embodiments, one or more RNA nucleotides in the compound is an unlocked nucleic acid (UNA), which lacks the C2′-C4′ bond normally found in ribonucleosides. UNAs are highly flexible, and are useful for fine-tuning the specificity and potency of the oligonucleotide. In various embodiments, the oligonucleotide comprises 1, 2, 3, 4, 5, or 6 UNA nucleotides. In some embodiments, 1, 2, 3, or 4 UNAs are used in the oligonucleotide along with LNAs.

In some embodiments, the compound is a FANA antisense oligonucleotides, that is, based on Fluoroarabinonucleic acid (i.e., 2′-deoxy-2′-fluoro-beta-D-arabinonucleic acid, or FANA). These nucleic acid analogs that can modulate gene expression by enzymatic degradation of a target RNA, which can be RNase H-mediated RNA cleavage.

In some embodiments, the antisense oligonucleotide has a modified backbone or modified internucleotide linkages. The term “internucleotide linkage” refers to the linkage between two adjacent nucleosides in a polynucleotide molecule. Naturally, the internucleotide linkage is a phosphodiester bond that forms between two oxygen atoms of the phosphate group and an oxygen atom of the sugar (either at 3′ or 5′ position) to form two ester bonds bridging between the two adjacent nucleosides. Modification of the internucleotide linkage may provide different characteristics, including but not limited to enhanced stability. For example, phosphorothioate or phosphorodithioate linkages increase the resistance of the internucleotide linkage to nucleases. Another example is phosphoacetate linkage (PACE), which may improve transfection characteristics and enhance nuclease resistance. Internucleotide linkages and oligonucleotide backbone modifications which may be employed in the oligonucleotides of the present description include, but are not limited to, phosphodiester, phosphorothioate, phosphorodithioate, methylphosphonate, alkylphosphonate, alkylphosphonothioate, phosphotriester, phosphoramidate, phosphoramidite, phosphorodiamidate, siloxane, carbonate, carboalkoxy, acetamidate, carbamate, morpholino, peptide nucleic acid, borano, thioether, bridged phosphoramidate, bridged methylene phosphonate, bridged phosphorothioate, and sulfone internucleoside linkages. In some embodiments, the antisense oligonucleotide comprises one or more phosphorothioate or phosphorodithioate nucleotides.

In some embodiments, the antisense oligonucleotide comprises one or more phosphorothioate or phosphorodithioate internucleotide linkages. In some embodiments, phosphorothioate or phosphorodithioate bonds can be introduced between the last three to five nucleotides at the 5′- and/or 3′-end of the oligonucleotide to reduce exonuclease degradation. In some embodiments, the antisense oligonucleotide has a combination of phosphodiester and phosphorothioate/phosphorodithioate linkages. In some embodiments, the antisense oligonucleotide contains at least two, at least three, at least four, at least five, at least six, at least seven, at least eight, at least nine, or at least ten phosphorothioate or phosphorodithioate internucleotide linkages. In some embodiments, the antisense oligonucleotide comprises substantially alternating phosphodiester and phosphorothioate internucleotide linkages. In some embodiments, the antisense oligonucleotide is fully phosphorothioate/phosphorodithioate linked (i.e., all bonds are either phosphorothioate or phosphorodithioate). In embodiments, the antisense oligonucleotide is fully phosphorothioate linked.

In some embodiments, such as where RNaseH recruitment is not desired, the antisense oligonucleotide has a morpholino backbone. Morpholino oligonucleotides do not generally trigger the degradation of their target RNA molecules, and can be effective for steric blocking of a target RNA sequence. Morpholino oligonucleotides and their synthesis are disclosed generally in U.S. Pat. Nos. 11,028,386, 10,947,533, and 10,927,378, each of which is hereby incorporated by reference in its entirety. In some embodiments, the antisense oligonucleotide comprises thiomorpholino nucleotides and/or other substituted or modified nucleotides such as those described, for example, in International Patent Application Publication No. WO/2019/060522 and International Patent Application Publication No. WO/2018/057430, each of which is hereby incorporated by reference in its entirety. For example, Langner et al. describe methods for synthesizing oligonucleotide analogs dubbed thiophosphoramidate morpholino oligonucleotides (TMOs) which incorporate morpholino nucleosides and phosphorothioate linkages (“Synthesis and characterization of thiophosphoramidate morpholino oligonucleotides and chimeras.” JACS 142.38 (2020): 16240-16253; see also Dumbović, Gabrijela, et al. “Nuclear compartmentalization of TERT mRNA and TUG1 lncRNA is driven by intron retention.” Nature Communications 12.1 (2021): 1-19; both of which are hereby incorporated by reference in their entireties). Thus, the antisense oligonucleotides described herein may comprise full or partial TMO-modified nucleotides, or may comprise chimeras of TMO-modified nucleotides and unmodified nucleotides and/or other nucleotides comprising different modifications (e.g., LNAs).

In some embodiments, the antisense oligonucleotide may contain one or more modified bases. In some embodiments, cytosine is replaced with 5-methylcytosine, which may enhance base pairing. Other modified bases (particularly of cytosine or guanine) can be employed to reduce immunogenicity, where needed. Other modified bases are described in U.S. Pat. No. 10,064,959, which is hereby incorporated by reference. In various embodiments, cytidine nucleobases in the antisense oligonucleotide are 5-methyl cytidine. Thus, it will be understood by the skilled person that where a sequence includes a cytidine nucleobase (“C”), the term includes 5-methyl C. Further, it is understood that where uracil bases are included in a sequence, the term “U” includes pseudouridine and N1-methylpseudouridine. Other modified versions of canonical bases are likely contemplated by the instant disclosure.

In some embodiments, the antisense oligonucleotide contains one or more Inosine bases, which in some embodiments are deoxyInosine, which can be employed in the central block of DNA nucleotides in gapmers. Inosine can take the place of any base, e.g., guanine, cytosine, adenine, or thymine nucleobase. In embodiments, the oligonucleotide has from one to four Inosine nucleobases, such as one, two, or three Inosine nucleobases.

The inosine modification can tune cleavage of the mRNA in some embodiments, and/or tune hybridization properties Exemplary antisense oligonucleotides according to the invention are shown in Table 2, which are 14-mer oligonucleotides with a 3-8-3 gapmer structure. Exemplary antisense oligonucleotides according to the invention are also shown in Table 5, having various lengths and gapmer structures. In embodiments, the antisense oligonucleotides are 13-mer oligonucleotides with a 3-8-2 gapmer. In embodiments, the antisense oligonucleotides are 14-mer oligonucleotides with a 3-9-2 gapmer. In embodiments, the antisense oligonucleotides are 13-mer oligonucleotides with a 2-8-3 gapmer. In embodiments, the antisense oligonucleotides are 14-mer oligonucleotides with a 2-9-3 gapmer. In embodiments, the antisense oligonucleotides are 15-mer oligonucleotides with a 3-10-2 gapmer. In embodiments, the antisense oligonucleotides are 17-mer oligonucleotides with a 2-12-3 gapmer. In embodiments, the antisense oligonucleotides are 20-mer oligonucleotides with a 2-15-3 gapmer. In embodiments, the antisense oligonucleotides are 17-mer oligonucleotides with a 3-11-3 gapmer. In embodiments, the antisense oligonucleotides are 16-mer oligonucleotides with a 3-10-3 gapmer. In embodiments, the antisense oligonucleotides are 19-mer oligonucleotides with a 3-13-3 gapmer. In embodiments, the antisense oligonucleotides are 18-mer oligonucleotides with a 3-12-3 gapmer. In embodiments, the antisense oligonucleotides are 18-mer oligonucleotides with a 3-13-2 gapmer.

In embodiments, the melting temperature of the antisense oligonucleotide hybridized to its target sequence is at least about 35° C. The Tₘ of an oligonucleotide is the temperature at which 50% of the oligonucleotide is duplexed with its perfect complement and 50% is free in solution. The Tm can be determined experimentally by measuring the absorbance change of the oligonucleotide with its complement as a function of temperature. The Tₘ can also be estimated using known publicly available Tₘ calculators. In some embodiments, the Tₘ of the oligonucleotide hybridized to its target sequence is at least about 40° C., or at least about 45° C., or at least about 50° C. In some embodiments, the Tₘ of the oligonucleotide hybridized to its target sequence is from about 35° C. to about 60° C. In some embodiments, the Tₘ of the oligonucleotide hybridized to its target sequence is from about 40° C. to about 60° C., or from about 50° C. to about 60° C.

In some embodiments, the antisense oligonucleotide further comprises a cell targeting or penetrating moiety, which in some embodiments is conjugated directly or indirectly to the 3′ end of the oligonucleotide, and optionally though a linker. In some embodiments, the composition further comprises a sterol conjugate (e.g., cholesterol conjugate) or fatty acid conjugate such as a palmitoyl or stearyl lipid conjugate, which is optionally conjugated to the 3′ end of the antisense oligonucleotide. These moieties can enhance cell penetration. See U.S. Pat. No. 9,012,225, which is hereby incorporated by reference in its entirety.

In embodiments, the cell targeting moiety is a N-acetylgalactosamine (GalNAc) ligand, which in embodiments can improve targeting of the compound to hepatocytes. Exemplary GalNAc ligands are described in U.S. Pat. Nos. 5,985,826 and 8,552,163, which are hereby incorporated by reference in their entireties.

In various embodiments, the targeting or cell penetrating moiety comprises an antibody or antigen-binding fragment thereof, an aptamer, a peptide, a biological ligand (e.g., including a glycoconjugate), lipid, sterol, cholesterol or derivative thereof, integrin, RGD peptide, or cell-penetrating peptide (CPP). More specifically, the targeting or cell penetrating moiety may be selected from a single-domain antibody, a single chain antibody, a bi-specific antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, and a peptide mimetic molecule. Various ligand-binding platforms are described in US Patent Nos. or Patent Publication Nos. U.S. Pat. No. 7,417,130, US 2004/132094, US 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties.

In some embodiments, the cell targeting moiety is an aptamer, which can be an aptamer described in U.S. Pat. Nos. 9,464,293; 10,550,394, or U.S. Pat. No. 11,261,449, which are hereby incorporated by reference in their entireties. In embodiments, the cell targeting moiety targets the antisense oligonucleotide to pancreas.

In another aspect, the present disclosure provides a pharmaceutical composition comprising a compound of the instant disclosure, and a pharmaceutically acceptable vehicle. In some embodiments, the composition comprises the antisense oligonucleotide encapsulated in a particle, such as a liposome, polymeric nanoparticle, lipid nanoparticle (LNP), or exosome. Exemplary polymeric nanoparticles can be formed of PLA, PLGA, or PEG copolymers thereof. In some embodiments, the particle comprises poly(P amino ester) polymers. In various embodiments, the particle comprises di- and tri-block copolymers, as described for example in U.S. Pat. Nos. 9,476,063; 9,505,867; and US Patent Application Publication No. 2017/0049801, which are each hereby incorporated by reference in their entireties. In various embodiments, the LNPs comprise a cationic or ionizable lipid, a neutral lipid, a structural lipid, and a PEGylated lipid.

In some embodiments, the compound is incorporated into an LNP that comprises a cationic or ionizable lipids, such as but not limited to those known as ALC-059 or SM-102. Other exemplary lipids and LNP compositions are described in U.S. Pat. No. 9,708,628 and US 2021/0023008, which are hereby incorporated by reference in their entireties.

Exemplary structural lipids can be selected from one or more of cholesterol, fecosterol, sitosterol, ergosterol, campesterol, stigmasterol, brassicasterol, tomatidine, ursolic acid, and tocopherols (e.g., alpha tocopherol). In some embodiments, the structural lipid is cholesterol.

In some embodiments, the LNP comprises one or more phospholipids. Exemplary phospholipids are selected from cardiolipins, sterol modified lipids (modified with a cholesterol moiety attached at the sn-2 carbon of the glycerol backbone), mixed-acyl glycerophospholipids, and symmetrical acyl glycerophospholipids. Head groups for acyl glycerophospholipids include, for example, phosphatidic acid, lysophosphatidic acid, phosphatidylcholine, phosphatidylethanolamine, phosphatidylglycerol, phosphoinositides, and phosphatidylserine. Exemplary phospholipids are selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the lipid nanoparticle further comprises one or more PEG lipids. A PEG lipid is a lipid modified with polyethylene glycol. Exemplary PEG lipids are selected from one or more of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol. A PEG lipid may be selected from PEG-c-DOMG, PEG-DMG, PEG-DLPE, PEG-DMPE, PEG-DPPC, PEG-Cholesterol, PEG tocopherol, and a PEG-DSPE lipid.

Lipid particle formulations that find use with embodiments of the present disclosure include those described in U.S. Pat. Nos. 9,738,593; 10,221,127; 10,166,298, which are hereby incorporated by reference in their entireties. In some embodiments, the liposomes or nanoparticles further comprise a targeting moiety (e.g., as described), targeting the LNP or other particle to a desired tissue or cell type. In embodiments, the cell targeting moiety is conjugated to a portion of the PEG groups.

In various embodiments, the antisense oligonucleotide is contained with exosomes for delivery. Exemplary exosomes and their preparation and/or use are described in U.S. Pat. No. 10,851,372; U.S. Patent Application Publication No. 2018/0193270; and U.S. Patent Application Publication No. 2020/00317746, which are hereby incorporated by reference in their entireties.

In various embodiments, the pharmaceutical composition is formulated for parenteral administration. For example, the composition may be formulated for a route selected from intravenous, subcutaneous, intradermal, intralymphatic, intramuscular, intratumoral, or intrathecal administration, or other suitable parenteral route. In some embodiments, other routes, including routes for local delivery closer to site of injury or inflammation are employed, such as but not limited to intranasal administration, topical administration, and pulmonary administration.

In another aspect, the present disclosure provides a method for treating a disease or condition associated with NLRP3 and/or NLRP1 expression or activation of the NLRP3 and/or NLRP1 inflammasome. In various embodiments, the method comprises administering an antisense oligonucleotide or pharmaceutical composition of the present disclosure to the subject. The subject is generally a mammal, such as a human. In embodiments, the disease or condition manifests as inflammation of one or more tissues, organs, or systems, such as central nervous system (e.g., brain), skeletal muscle, liver, pancreas, gastrointestinal system, lung, heart, skin, eyes, ears, mucosal membranes, joints, adipose tissue, etc. Compositions can be systemically or locally administered according to methods known in the art.

Dose, frequency, and duration of administration can be adjusted according to the needs to the subject. In some embodiments, the compositions are administered about daily, about weekly, about bimonthly (i.e., about every other week), about monthly, or about quarterly. In some embodiments, administration dose and frequency is determined based on the patient's condition or response. In some embodiments, the composition is administered at least once per month or at least once per week. In some embodiments, the subject receives at least 4 or at least 8, or at least 12 administrations of the composition. Compositions can be administered according to a route (either systemic or local) as already described.

In various embodiments, the disease or condition associated with NLRP3 and/or NLRP1 expression or activation of the NLRP3 and/or NLRP1 inflammasome comprises an inflammatory disease, an autoimmune disease, or neurodegenerative disease. For example, in some embodiments, the subject has a neurodegenerative disease, such as amyotrophic lateral sclerosis (ALS), Parkinson's disease, Alzheimer's disease, Huntington's disease, or multiple sclerosis.

In other embodiments, the disease or condition is an inflammatory disease or condition such as migraine, traumatic brain injury, stroke (e.g., acute ischemic stroke), myocardial infarction, viral or bacterial meningitis, or nerve injury or inflammation (e.g., neuritis). In various embodiments, the nerve injury comprises a peripheral nerve injury and/or a spinal cord injury.

In still other embodiments, the disease or condition is an autoimmune disease. Exemplary autoimmune diseases include but are not limited to diabetes mellitus, inflammatory bowel syndrome (IBD) (ulcerative colitis or Crohn's disease), arthritis (e.g., rheumatoid arthritis or osteoarthritis), multiple sclerosis, lupus, and dermatomyositis.

Other conditions or diseases that may be treated according to the instant disclosure include diseases or conditions associated with the immune system, the cardiovascular system, the endocrine system, the gastrointestinal tract, the renal system, the respiratory system, the central nervous system, cancer, or pathogen infection (e.g., infection by virus, bacterium, protist, worm, or fungus).

Non-limiting examples of viruses include influenza virus, cytomegalovirus, Epstein Barr Virus, human immunodeficiency virus (HIV), alphavirus such as Chikungunya and Ross River virus, flaviviruses such as Dengue virus, Zika virus, or papillomavirus. In embodiments, the virus is SARS-CoV-2 (e.g., in embodiments, the subject may have long covid). Non-limiting examples of pathogenic bacteria include Staphylococcus aureus, Helicobacter pylon, Bacillus anthracis, Bordatella pertussis, Corynebacterium dipthenae, Clostridium tetani, Clostridium botulinum, Streptococcus pneumoniae, Streptococcus pyogenes, Listeria monocytogenes, Hemophilus influenzae, Pasteureiia multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi, or Yersinia pestis. Non-limiting examples of protists include Plasmodium (e.g., cerebral malaria), Babesia, Giardia, Entamoeba, Leishmania, or Trypanosomas. Non-limiting examples of worms include helminths inclusive of schistisimes, roundworms, tapeworms, or flukes. Non-limiting examples of fungi include Candida or Aspergillus species.

In some embodiments, the disease or condition is caused at least partially by constitutively active inflammation, such as: cryopyrin-associated periodic syndromes (CAPS); Muckle-Wells syndrome (MWS); familial cold automflammatory syndrome (FCAS); or neonatal-onset multisystem inflammatory disease (NOMID).

In some embodiments, the disease or condition is an autoinflammatory disease, such as: familial Mediterranean fever (FMF); TNF receptor associated periodic syndrome (TRAPS); mevalonate kinase deficiency (MET); hyperimmunoglobulinemia D and periodic fever syndrome; deficiency of interleukin 1 receptor (DIRA) antagonist; Majeed syndrome; pyogenic arthritis; pyoderma gangrenosum and acne (PAPA); haploinsufficiency of A20 (HA20); pediatric granulomatous arthritis (PGA); PLCG2-associated antibody deficiency and immune dysregulation (PLAID); PLCG2-associated autoinflammation; antibody deficiency and immune dysregulation (APLAID); sideroblastic anemia with B-cell immunodeficiency; periodic fevers, and developmental delay (SIFD); Sweet syndrome; chronic nonbacterial osteomyelitis (CNO); chronic recurrent multifocal osteomyelitis (CRMO); synovitis, acne, pustulosis, hyperostosis, or osteitis syndrome (SAPHO).

In some embodiments, the disease or condition is an autoimmune disease, such as multiple sclerosis (MS); type I diabetes; psoriasis; rheumatoid arthritis; Behcet's disease; Sjogren's syndrome; or Schnitzler syndrome.

In some embodiments, the disease or condition is a respiratory disease, such as idiopathic pulmonary fibrosis (IPF); chronic obstructive pulmonary disorder (COPD); asthma; asbestosis; neutrophilic asthma; or silicosis and cystic fibrosis. In embodiments, the disease or condition is neutrophilic asthma.

In some embodiments, the disease or condition is a metabolic disease, such as type II diabetes; atherosclerosis; obesity; metabolic syndrome, gout; or pseudo-gout.

In some embodiments, the disease or condition is an ocular disease such as ocular epithelium, age-related macular degeneration (AMD) (wet or dry), diabetic macular edema, corneal infection, uveitis, or dry eye.

In some embodiments, the disease or condition is a kidney disease such as chronic kidney disease, oxalate nephropathy, or diabetic nephropathy.

In some embodiments, the disease or condition is a liver disease such as non-alcoholic steatohepatitis (NASH), alcoholic liver disease (ASH), hepatitis (e.g., HCV), or cirrhosis.

In some embodiments, the disease or condition is an inflammatory reaction in skin such as contact hypersensitivity or sunburn.

In some embodiments, the disease or condition is an inflammatory reaction in the joints such as osteoarthritis, systemic juvenile idiopathic arthritis, adult-onset Still's disease, or relapsing polychondritis.

In some embodiments, the disease or condition is polymyositis, stroke, myocardial infarction, Graft versus Host Disease, hypertension, colitis, celiac disease, abdominal aortic aneurism, wound healing, depression, psychological stress, pericarditis including Dressler's syndrome, or ischemia reperfusion injury.

In some embodiments, the disease or condition is cancer, such as a solid tumor or hematological malignancy (e.g., a leukemia such as AML).

As used herein, the term “about,” unless the context requires otherwise, means ±10% of an associated value.

All publications, patents, and patent applications mentioned in this specification are herein incorporated by reference to the same extent as if each individual publication, patent, or patent application was specifically and individually indicated to be incorporated by reference.

Other aspects and embodiments of the present disclosure will be apparent from the following Examples.

### EXAMPLES

### Example 1: Suppression of NLRP3 in THP-1 Human Monocyte Cells Following ASO Treatment

14-mer antisense oligonucleotides containing LNA modifications and fully phosphorothioated backbone were synthesized (Integrated DNA Technologies, Inc). Each oligo was transfected into THP-1 human monocyte cell line (ATCC, pre-differentiated with 300 ng/mL phorbol 12-myristate 13-acetate) at 100 nM, 40 nM, and 16 nM final concentration using 0.4 uL per well of Lipofectamine RNAiMax (Thermo-Fisher) in a 96 well format. After 24 hours, the cells were lysed with 0.05 mL per well of QUANTIGENE lysis mixture (Life Technologies) for gene expression assays. NLRP3 and PPIB housekeeping gene expression were assayed using the QUANTIGENE SINGLEPLEX assay kit (Life Technologies) according to the manufacturer's protocol for RNA capture, followed by subsequent signal amplification and detection by chemiluminescent reaction. The relative gene expression that is detected per well is quantified in relative light units (RLU) on the Spark luminometer (Tecan). The extent of NLRP3 knockdown is determined by the following formula:

$$\% {KD}_{sequence X} = 1 - [ ( RLU NLRP {3}_{sequence X} / RLU {PPIB}_{sequence X} ) / ( RLU NLRP {3}_{mock control} / RLU {PPIB}_{mock control} ) ]$$

TABLE 1
lists the nucleobase sequences assigned SEQ ID NO:
1 to 36.

| ASO Nucleobase Sequence | SEQ ID NO: |
|---|---|
| TGGAATATATCGAG | SEQ ID NO: 1 |
| GGAATATATCGAGC | SEQ ID NO: 2 |
| GAATATATCGAGCA | SEQ ID NO: 3 |
| AACTATTAAGCAAC | SEQ ID NO: 4 |
| ACTATTAAGCAACG | SEQ ID NO: 5 |
| CTATTAAGCAACGG | SEQ ID NO: 6 |
| GTATTACAGTTTAC | SEQ ID NO: 7 |
| TATTACAGTTTACG | SEQ ID NO: 8 |
| ATTACAGTTTACGG | SEQ ID NO: 9 |
| CCTTTTCGAATTTG | SEQ ID NO: 10 |
| CTTTTCGAATTTGC | SEQ ID NO: 11 |
| TTTTCGAATTTGCC | SEQ ID NO: 12 |
| CTTAATTTCTTCTC | SEQ ID NO: 13 |
| ACTTAATTTCTTCT | SEQ ID NO: 14 |
| AACTTAATTTCTTC | SEQ ID NO: 15 |
| CAACTTAATTTCTT | SEQ ID NO: 16 |
| GCAACTTAATTTCT | SEQ ID NO: 17 |
| TGCAACTTAATTTC | SEQ ID NO: 18 |
| TTGCAACTTAATTT | SEQ ID NO: 19 |
| CTTGCAACTTAATT | SEQ ID NO: 20 |
| TCTTGCAACTTAAT | SEQ ID NO: 21 |
| ATCTTGCAACTTAA | SEQ ID NO: 22 |
| AGATCTTGCAACTT | SEQ ID NO: 23 |
| AGAGATCTTGCAAC | SEQ ID NO: 24 |
| TGAGAGATCTTGCA | SEQ ID NO: 25 |
| GCTGAGAGATCTTG | SEQ ID NO: 26 |
| TTGCTGAGAGATCT | SEQ ID NO: 27 |
| TTTGCTGAGAGATC | SEQ ID NO: 28 |
| ATTTGCTGAGAGAT | SEQ ID NO: 29 |
| GATTTGCTGAGAGA | SEQ ID NO: 30 |
| TGATTTGCTGAGAG | SEQ ID NO: 31 |
| CTGATTTGCTGAGA | SEQ ID NO: 32 |
| CCTGATTTGCTGAG | SEQ ID NO: 33 |
| GCCTGATTTGCTGA | SEQ ID NO: 34 |
| AGCCTGATTTGCTG | SEQ ID NO: 35 |
| CAGCCTGATTTGCT | SEQ ID NO: 36 |

TABLE 2
Lists the chemically modified ASOs targeting NLRP3 mRNA

| Oligo | | Nucleobase |
|---|---|---|
| Identifier | ASO Sequence | sequence |
| STN-100867 | +T*+G*+G*A*A*T*A*T*A*T*C*+G*+A*+G | SEQ ID NO: 1 |
| STN-100868 | +G*+G*+A*A*T*A*T*A*T*C*G*+A*+G*+C | SEQ ID NO: 2 |
| STN-100869 | +G*+A*+A*T*A*T*A*T*C*G*A*+G*+C*+A | SEQ ID NO: 3 |
| STN-100870 | +A*+A*+C*T*A*T*T*A*A*G*C*+A*+A*+C | SEQ ID NO: 4 |
| STN-100871 | +A*+C*+T*A*T*T*A*A*G*C*A*+A*+C*+G | SEQ ID NO: 5 |
| STN-100872 | +C*+T*+A*T*T*A*A*G*C*A*A*+C*+G*+G | SEQ ID NO: 6 |
| STN-100873 | +G*+T*+A*T*T*A*C*A*G*T*T*+T*+A*+C | SEQ ID NO: 7 |
| STN-100874 | +T*+A*+T*T*A*C*A*G*T*T*T*+A*+C*+G | SEQ ID NO: 8 |
| STN-100875 | +A*+T*+T*A*C*A*G*T*T*T*A*+C*+G*+G | SEQ ID NO: 9 |
| STN-100876 | +C*+C*+T*T*T*T*C*G*A*A*T*+T*+T*+G | SEQ ID NO: 10 |
| STN-100877 | +C*+T*+T*T*T*C*G*A*A*T*T*+T*+G*+C | SEQ ID NO: 11 |
| STN-100878 | +T*+T*+T*T*C*G*A*A*T*T*T*+G*+C*+C | SEQ ID NO: 12 |
| STN-100879 | +C*+T*+T*A*A*T*T*T*C*T*T*+C*+T*+C | SEQ ID NO: 13 |
| STN-100880 | +A*+C*+T*T*A*A*T*T*T*C*T*+T*+C*+T | SEQ ID NO: 14 |
| STN-100881 | +A*+A*+C*T*T*A*A*T*T*T*C*+T*+T*+C | SEQ ID NO: 15 |
| STN-100882 | +C*+A*+A*C*T*T*A*A*T*T*T*+C*+T*+T | SEQ ID NO: 16 |
| STN-100883 | +G*+C*+A*A*C*T*T*A*A*T*T*+T*+C*+T | SEQ ID NO: 17 |
| STN-100884 | +T*+G*+C*A*A*C*T*T*A*A*T*+T*+T*+C | SEQ ID NO: 18 |
| STN-100885 | +T*+T*+G*C*A*A*C*T*T*A*A*+T*+T*+T | SEQ ID NO: 19 |
| STN-100886 | +C*+T*+T*G*C*A*A*C*T*T*A*+A*+T*+T | SEQ ID NO: 20 |
| STN-100887 | +T*+C*+T*T*G*C*A*A*C*T*T*+A*+A*+T | SEQ ID NO: 21 |
| STN-100888 | +A*+T*+C*T*T*G*C*A*A*C*T*+T*+A*+A | SEQ ID NO: 22 |
| STN-100889 | +A*+G*+A*T*C*T*T*G*C*A*A*+C*+T*+T | SEQ ID NO: 23 |
| STN-100890 | +A*+G*+A*G*A*T*C*T*T*G*C*+A*+A*+C | SEQ ID NO: 24 |
| STN-100891 | +T*+G*+A*G*A*G*A*T*C*T*T*+G*+C*+A | SEQ ID NO: 25 |
| STN-100892 | +G*+C*+T*G*A*G*A*G*A*T*C*+T*+T*+G | SEQ ID NO: 26 |
| STN-100893 | +T*+T*+G*C*T*G*A*G*A*G*A*+T*+C*+T | SEQ ID NO: 27 |
| STN-100894 | +T*+T*+T*G*C*T*G*A*G*A*G*+A*+T*+C | SEQ ID NO: 28 |
| STN-100895 | +A*+T*+T*T*G*C*T*G*A*G*A*+G*+A*+T | SEQ ID NO: 29 |
| STN-100896 | +G*+A*+T*T*T*G*C*T*G*A*G*+A*+G*+A | SEQ ID NO: 30 |
| STN-100897 | +T*+G*+A*T*T*T*G*C*T*G*A*+G*+A*+G | SEQ ID NO: 31 |
| STN-100898 | +C*+T*+G*A*T*T*T*G*C*T*G*+A*+G*+A | SEQ ID NO: 32 |
| STN-100899 | +C*+C*+T*G*A*T*T*T*G*C*T*+G*+A*+G | SEQ ID NO: 33 |
| STN-100900 | +G*+C*+C*T*G*A*T*T*T*G*C*+T*+G*+A | SEQ ID NO: 34 |
| STN-100901 | +A*+G*+C*C*T*G*A*T*T*T*G*+C*+T*+G | SEQ ID NO: 35 |
| STN-100902 | +C*+A*+G*C*C*T*G*A*T*T*T*+G*+C*+T | SEQ ID NO: 36 |

“+” designates a Locked Nucleic Acid (LNA) nucleotide, but which in embodiments is replaced with 2′-OMe, 2′-MOE, or other bridged nucleotide such as cEt.
“*” designates a phosphorothioate linkage

TABLE 3
NLRP3 mRNA knockdown in THP-1 human monocyte cells following treatment with
antisense oligonucleotides at concentrations of 100 nM, 40 nM, and 16 nM.

| | % KD | | | | | |
|---|---|---|---|---|---|---|
| | 100 nM | | 40 nM | | 16 nM | |
| Sequence Identifer | Mean | % CV | Mean | % CV | Mean | % CV |
| STN-100867 | 70.07% | 2.22% | 61.60% | 4.86% | 35.52% | 8.39% |
| STN-100868 | 79.80% | 2.91% | 65.60% | 5.20% | 33.51% | 16.90% |
| STN-100869 | 81.98% | 3.12% | 66.86% | 8.50% | 30.60% | 17.73% |
| STN-100870 | 21.90% | 6.88% | 10.70% | 38.68% | −8.64% | −96.39% |
| STN-100871 | 70.01% | 4.09% | 43.34% | 10.41% | 32.26% | 9.98% |
| STN-100872 | 71.58% | 0.41% | 54.50% | 4.77% | 32.32% | 10.59% |
| STN-100873 | 40.27% | 3.53% | 24.13% | 6.37% | −0.53% | −1684.4% |
| STN-100874 | 49.21% | 2.14% | −5.27% | −73.22% | −6.75% | −65.93% |
| STN-100875 | 43.67% | 4.66% | 22.04% | 4.13% | 0.96% | 26.00% |
| STN-100876 | 66.13% | 1.27% | 26.95% | n/a | −3.31% | −97.15% |
| STN-100877 | 68.98% | 6.23% | 29.57% | 2.53% | −6.86% | −55.89% |
| STN-100878 | 68.36% | 3.30% | 43.35% | 2.43% | 6.00% | 90.70% |
| STN-100879 | 48.82% | 0.08% | 36.69% | 0.63% | 25.00% | 6.88% |
| STN-100880 | 43.58% | 2.26% | 40.08% | 0.78% | 26.51% | 5.86% |
| STN-100881 | 46.68% | 3.95% | 39.34% | 3.21% | 31.10% | 4.22% |
| STN-100882 | 49.21% | 4.15% | 30.63% | 30.19% | 25.21% | 12.83% |
| STN-100883 | 56.01% | 12.26% | 34.48% | 4.30% | 17.38% | 48.70% |
| STN-100884 | 65.06% | 4.25% | 38.19% | 13.59% | 11.11% | 46.89% |
| STN-100885 | 57.55% | 8.52% | 36.06% | 3.52% | 24.76% | 19.98% |
| STN-100886 | 66.32% | 7.10% | 43.77% | 6.60% | 37.18% | 16.34% |
| STN-100887 | 39.76% | 12.64% | 22.31% | 52.01% | 24.47% | 27.47% |
| STN-100888 | 46.56% | 3.35% | 37.69% | 18.29% | 25.87% | 6.99% |
| STN-100889 | 55.88% | 3.61% | 34.78% | 23.97% | 27.13% | 26.50% |
| STN-100890 | 58.09% | 3.92% | 37.05% | 16.61% | 29.72% | 27.12% |
| STN-100891 | 37.89% | 3.19% | 30.97% | 31.34% | 27.05% | 6.34% |
| STN-100892 | 47.67% | 9.59% | 21.48% | 1.06% | 34.07% | 2.43% |
| STN-100893 | 40.13% | 11.55% | 14.12% | 143.52% | 28.09% | 1.43% |
| STN-100894 | 20.59% | 3.51% | −2.60% | −24.36% | −1.94% | −44.24% |
| STN-100895 | −2.38% | −340% | −16.10% | −51.93% | −6.71% | n/a |
| STN-100896 | 25.67% | 41.56% | 24.41% | 15.64% | 16.69% | 32.28% |
| STN-100897 | 38.18% | 7.55% | 8.72% | 145.85% | 22.19% | 9.42% |
| STN-100898 | −4.39% | −177.42% | 10.81% | 2.97% | 18.95% | 8.89% |
| STN-100899 | 0.43% | 2833% | 15.17% | 70.61% | 22.73% | 13.05% |
| STN-100900 | 13.57% | 99.70% | 15.66% | 9.29% | 23.14% | 4.49% |
| STN-100901 | 1.52% | 279.46% | 10.41% | 29.94% | 24.34% | 35.29% |
| STN-100902 | −26.58% | −20.91% | 20.79% | 191.73% | −6.29% | −250.20% |
| STN-100146 | 65.10% | 1.66% | 62.30% | 3.16% | 54.66% | 3.75% |
| STN-100121 | 49.50% | 1.79% | 56.82% | 0.22% | 17.87% | 26.67% |

### Example 2: Suppression of NLRP1 and NLRP3 in U87-MG Cell Line Following ASO Treatment

Antisense oligonucleotides containing LNA and fully phosphorothioated backbone (as disclosed in Table 5) were synthesized by Integrated DNA Technologies, Inc. Certain oligonucleotides includes from one to three deoxyInosine bases. Each oligo was transfected into U87-MG glioblastoma cell line (ATCC) at 100 nM, 25 nM, and 6.25 nM final concentration using 0.4 uL per well of Lipofectamine RNAiMax (Thermo-Fisher) in a 96 well format. After 24 hours, the cells were lysed with 0.05 mL per well of Quantigene lysis mixture (Life Technologies) for gene expression assays. NLRP1, NLRP3, and PPIB housekeeping gene expression were assayed using the Quantigene singleplex assay kit (Life Technologies) according to the manufacturer's protocol for RNA capture, followed by subsequent signal amplification and detection by chemiluminescent reaction. The relative gene expression that is detected per well is quantified in relative light units (RLU) on the Spark luminometer (Tecan). The extent of NLRP1 (or NLRP3) knockdown is determined by the following formula:

$$\% {KD}_{sequence X} = 1 - [ ( RLU {NLRP}_{sequence X} / RLU {PPIB}_{sequence X} ) / ( RLU {NLRP}_{mock control} / RLU {PPIB}_{mock control} ) ]$$

Tables 5 and 6 demonstrate the extent of NLRP1 and NLRP3 mRNA reduction in the U87-MG cell line following treatment with antisense oligonucleotides.

TABLE 4
Sequences of NLRP1/3 targeting antisense
oligonucleotides

| ASO Nucleobase Sequence | SEQ ID NO: |
|---|---|
| AGGCAGGCAGAXXAC | SEQ ID NO: 37 |
| CTXCCTGGCCAGXXTTG | SEQ ID NO: 38 |
| GAXGCCTCGGGAAG | SEQ ID NO: 39 |
| TCATCXGXGAAXTA | SEQ ID NO: 40 |
| GTGGTGGTCTTGGA | SEQ ID NO: 41 |
| TCXGCAGCCAXAGAGCAGAG | SEQ ID NO: 42 |
| GCAGCCAXAGAGCAGAG | SEQ ID NO: 43 |
| CCATGXTTCCTGAGGTC | SEQ ID NO: 44 |
| GTGXATGAAGCTGTAG | SEQ ID NO: 45 |
| ACATXGCXGCAAAGAACTC | SEQ ID NO: 46 |
| GCAAAGAACTCXTGGAAA | SEQ ID NO: 47 |
| CAGGCCXAAXAGGAAACGT | SEQ ID NO: 48 |
| TCTCGTACAAXCAGTXGA | SEQ ID NO: 49 |
| AGXACXGAXAAGAG | SEQ ID NO: 50 |
| CTCAGGTCCAGCTC | SEQ ID NO: 51 |
| AGXTCCTGXCAXCA | SEQ ID NO: 52 |

“X” designates deoxyinosine

TABLE 5
Sequences of NLRP1/3 targeting antisense oligonucleotides

| Oligo | | Nucleobase |
|---|---|---|
| Identifier | ASO Sequence | sequence |
| STN-101008 | +A*+G*+G*C*A*G*G*C*A*G*A*/ideoxyI//ideoxyI/+A*+C | SEQ ID NO: 37 |
| STN-101010 | +C*+T*/ideoxyI/C*C*T*G*G*C*C*A*G*/ideoxyI//ideoxy | SEQ ID NO: 38 |
| | I/+T*+T*+G | |
| STN-101011 | +G*+A*/ideoxyI/G*C*C*T*C*G*G*G*+A*+A*+G | SEQ ID NO: 39 |
| STN-101012 | +T*+C*+A*T*C*/ideoxyI/G*/ideoxyI/G*A*A*/ideoxyI/+T | SEQ ID NO: 40 |
| | *+A | |
| STN-101013 | +G*+T*+G*G*T*G*G*T*C*T*T*+G*+G*+A | SEQ ID NO: 41 |
| STN-101014 | +T*+C*/ideoxyI/G*C*A*G*C*C*A*/ideoxyI/A*G*A*G* | SEQ ID NO: 42 |
| | C*A*+G*+A*+G | |
| STN-101015 | +G*+C*+A*G*C*C*A*/ideoxyI/A*G*A*G*C*A*+G*+A | SEQ ID NO: 43 |
| | *+G | |
| STN-101016 | +C*+C*+A*T*G*/ideoxyI/T*T*C*C*T*G*A*G*+G*+T*+C | SEQ ID NO: 44 |
| STN-101017 | +G*+T*+G*/ideoxyI/A*T*G*A*A*G*C*T*G*+T*+A*+G | SEQ ID NO: 45 |
| STN-101018 | +A*+C*+A*T*/ideoxyI/G*C*/ideoxyI/G*C*A*A*A*G*A | SEQ ID NO: 46 |
| | *A*+C*+T*+C | |
| STN-101019 | +G*+C*+A*A*A*G*A*A*C*T*C*/ideoxyI/T*G*G*+A*+ | SEQ ID NO: 47 |
| | A*+A | |
| STN-101020 | +C*+A*+G*G*C*C*/ideoxyI/A*A*/ideoxyI/A*G*G*A*A | SEQ ID NO: 48 |
| | *A*+C+G*+T | |
| STN-101021 | +T*+C*+T*C*G*T*A*C*A*A*/ideoxyI/C*A*G*T*/ideox | SEQ ID NO: 49 |
| | yI/+G*+A | |
| STN-101022 | +A*+G*/ideoxyI/A*C*/ideoxyI/G*A*/ideoxyI/A*A*+G*+ | SEQ ID NO: 50 |
| | A*+G | |
| STN-101023 | +C*+T*+C*A*G*G*T*C*C*A*G*+C*+T*+C | SEQ ID NO: 51 |
| STN-101024 | +A*+G*/ideoxyI/T*C*C*T*G*/ideoxyI/C*A*/ideoxyI/+C*+A | SEQ ID NO: 52 |

“+” designates a Locked Nucleic Acid (LNA) nucleotide, but which in embodiments is replaced with 2′-OMe, 2′-MOE, or other bridged nucleotide such as cEt.
“+” designates a phosphorothioate linkage.
“/ideoxyI/” designates deoxyinosine

TABLE 6
NLRP3 mRNA knockdown in U87-MG cell line following treatment with ASO

| | % NLRP3 KD | | | | | |
|---|---|---|---|---|---|---|
| | 100 nM | | 25 nM | | 6.25 nM | |
| Sequence Name | Mean | % CV | Mean | % CV | Mean | % CV |
| STN-101008 | 65.0% | 1.9% | 9.4% | 0.7% | 0.1% | 1350% |
| STN-101010 | 45.6% | 9.9% | −18.2% | −23.2% | −17.2% | −4.6% |
| STN-101011 | 14.6% | 21.6% | −0.8% | −494.6% | −10.5% | −21.7% |
| STN-101012 | 11.1% | 25.5% | −5.0% | −7.8% | −4.4% | −15.6% |
| STN-101013 | 83.0% | 2.5% | 11.0% | 31.7% | −0.8% | −208.5% |
| STN-101014 | 11.5% | 59.4% | −10.0% | −16.7% | −5.8% | −40.2% |
| STN-101015 | 78.6% | 0.5% | 39.6% | 8.1% | −3.4% | −75.2% |
| STN-101016 | 86.8% | 0.6% | 79.7% | 2.3% | 39.7% | 2.8% |
| STN-101017 | 77.9% | 1.8% | 67.9% | 0.7% | 35.0% | 5.1% |
| STN-101018 | 75.1% | 2.6% | 39.5% | 4.3% | 6.5% | 24.8% |
| STN-101019 | 57.8% | 7.9% | 36.5% | 8.9% | 10.9% | 10.5% |
| STN-101020 | 83.6% | 0.4% | 69.9% | 0.9% | 26.5% | 5.9% |
| STN-101021 | 40.9% | 7.9% | 4.1% | 0.0% | 2.7% | 65.4% |
| STN-101022 | 11.5% | 53.0% | −10.8% | −135.9% | −0.2% | −1087% |
| STN-101023 | 47.4% | 8.9% | 5.1% | 40.7% | −4.9% | −14.8% |
| STN-101024 | −7.1% | −66.1% | −2.1% | −74.6% | −0.5% | −124% |
| STN-100146 | 84.3% | 4.8% | 73.2% | 9.5% | 59.5% | 1.4% |
| (NLRP3 only) | | | | | | |
| Nontarget Oligo | −31.2% | −9.6% | −29.9% | −30.6% | −6.4% | −0.1% |

TABLE 7
NLRP1 mRNA knockdown in U87-MG cell line following treatment with ASO

| | % NLRP1 KD | | | | | |
|---|---|---|---|---|---|---|
| | 100 nM | | 25 nM | | 6.25 nM | |
| Sequence Name | Mean | % CV | Mean | % CV | Mean | % CV |
| STN-101008 | 68.8% | 0.4% | 8.0% | 47.7% | 1.1% | 773% |
| STN-101010 | 76.3% | 1.6% | 48.4% | 3.3% | 11.7% | 33.1% |
| STN-101011 | 63.6% | 2.9% | 7.9% | 29.2% | −2.8% | −55.4% |
| STN-101012 | −7.5% | −207.5% | −35.8% | −0.2% | −6.9% | −20.7% |
| STN-101013 | 90.7% | 0.6% | 70.1% | 5.9% | 43.5% | 5.2% |
| STN-101014 | 49.3% | 3.7% | 9.6% | 17.5% | −2.7% | −150% |
| STN-101015 | 66.5% | 0.7% | 70.3% | 0.5% | 40.9% | 6.2% |
| STN-101016 | 49.1% | 4.0% | 54.7% | 4.1% | 18.9% | 5.0% |
| STN-101017 | 90.3% | 0.4% | 73.7% | 0.9% | 47.2% | 3.7% |
| STN-101018 | 62.3% | 6.4% | 14.0% | 64.1% | −10.7% | −38.5% |
| STN-101019 | 84.3% | 4.4% | 75.5% | 3.6% | 32.2% | 3.4% |
| STN-101020 | 72.0% | 0.5% | 68.3% | 0.2% | 35.9% | 5.2% |
| STN-101021 | 57.4% | 0.2% | 27.3% | 0.0% | 7.8% | 4.3% |
| STN-101022 | 1.5% | 11.5% | −19.1% | −70.7% | −8.0% | −18.7% |
| STN-101023 | 65.8% | 5.6% | 51.5% | 6.7% | 26.8% | 21.4% |
| STN-101024 | 17.6% | 20.5% | −1.3% | −7.9% | −4.6% | −99.6% |
| STN-100146 | 14.4% | 33.7% | −27.0% | −122% | −1.0% | −456% |
| (NLRP3 only) | | | | | | |
| Nontarget Oligo | 15.1% | 59.1% | −9.0% | −105% | −8.1% | −27.2% |

TABLE 8
NLRP3 (SEQ ID NO: 53 to 59) and NLRP1 (SEQ ID NOs: 60 to 65) mRNA
Sequences

| SEQ ID | |
|---|---|
| NO: | Nucleic acid sequence |
| 53 | GCATTTCCTCTCTAGCTGTTCCTGAGGCTGGCATCTGGGTAAGTCCAGCTCCGCGGG |
| | GCAGTGTGGGCGACAGGACCGGGAGAGCAGTGGGGGAGATTGTGTTCAGAGGGCA |
| | GAACTTGTCCTGGTTAATGCACTAGGAATTTCTTCTGGCGTCTCCAAGGTTGTGCTTC |
| | AACAACATTTCTGGAATGTTCTTTTATCATTATTTGGGCTCTACGTGTGCAAGTTCGT |
| | GTGTGTGTATGTGGCGGGGGGAAGCGGGGAGGACAGCGGCAGCAACCATAGTTTAC |
| | ACCCACGACTTCAGCAGATTTGCCCAGTATCCCTCCCAGGGGAGAAGATGGATGGG |
| | ATATACAGAAGGAAACAAACCTCCTTAAAGACCAAAGGGCAGCAGATTTTTGTGCT |
| | GGTGTTAGGGTGGAAGGAGCACAGAAGAAAGGCAGAATCCTGATGCAGATGGAAG |
| | GACTGATGGGGCAGGAGCTGGGAGGGTATGCGGAGCTAATAGGGCTGTCTGGAGA |
| | GCAGTCACCTCAGGATGGGCAGGAGCTGGGAGGGTATGCGGAGCTAATAGGGCTGT |
| | CTGGAGAGCAGTCACCTCAGGATGCACAGGGCTGGGTTTGCAGGGCTCAGGAGGCG |
| | AGGAGTAGATAGGCAGGAATGGAGATGGGACGGGAGAGATGAGTGAGTCTGAGGC |
| | ATCTTGACATTGAACTGCAGTCACAGAAATTCCAATTTGGAGAGCTCCACCGTTTTC |
| | CAGGCTCTCTGCCTCCCTTGAGTGTTATTTATGGCTTTAGGTTGAGGTGCTTTGCCAA |
| | ATAGAGTATAATCAAGGGATCAAGGAGGGAACAATTCCAAACAAAGCCAACTTGTT |
| | CATCCATCCGGACTCTAGGAGATATGGAAGATCTAAAGATACAGCAGGTGATTGCT |
| | AGTTTTTGTGGAACTCACAGTCTAGTTGGGAAGACTATGTTGGTGAATGCTGCAGTG |
| | ATACACCTGTGATAAATGCTGGGGAAGTGTGTCTTTTAGTCATCTATTTTGTTGTCTT |
| | TGTCTTTGTCTGACTGAAACAAGTGATGGAAGACATGGTCTCATATCTCTGGTCAAA |
| | ATCCTGGCTCTGTTCCTTCCTGGTTCTAAACCCCTCGGCAGGCTTCCTAGTCCCCCTA |
| | AGACTCAGTTTATGCATCTATAAAATGGGGCTAGGAATAGGTGCACCTCATAGAGC |
| | TCTGTGCAGGTTAGATGGGGAAAAACATCAGTGAGTGCCTAGCCTGTGGAAAGCTC |
| | TAAATTAATGTGAATTATAATGATAACAATTGCGAATTTTGCAAGGAGACATTAAA |
| | GGATGTATGTTTTTATTTATTTTATTTTATTCTATTTTATTTTTTGAGATGGAGTTTTG |
| | CTCTTATTGCCCAGGCTGGAGTGCAGTGGTGTGATCTTGGCTCATCGCAACCTCCGC |
| | CTCCTGGGTTCAAGCGATTCTCCTGCCTCAGCCTCCCGAGTAGCTGGGATTACAGGT |
| | GCCTGGTACCATGCCCGGTTAATTTTTGTATTTTTAGTAGAGACGGGATTTCTCCCTG |
| | TTGTTTGGTGAGCCTGGTCTCCAACTCCTGACCTCAGGTGATCCGCCCACCTCGGCC |
| | TCCCAAAGTGCTGGGATTACAGGGATGTATGTTTTTATTATTCTCCATTATGCACTCC |
| | CAGCTTCATCTCACTTCAATCCACTGGTTGATAGAAGTGCAGTCAAAGACTGTCCCC |
| | TCCTCTGCAAGCTTTTATGTTCTCTCTCCTCTCTCCCTAGCCACGTCCTGTTCTGTTAT |
| | CAACACACTTACTCACCTCCTGGCCTTCTCCTTCTATTGTCTCTCATCCCTCTTCCCTC |
| | ACAAAAACAGAAGCAAAGAGCCAGAGCCTTCAGTTTGGAGGAACTGAAAACATTCT |
| | CTTCTGCTTTCTCATTTTGTAGATGAGGAAACTGAAGTTGAGGAATAGTGAAGAGTT |
| | TGTCCAATGTCATAGCCCCGTAATCAACGGGACAAAAATTTTCTTGCTGATGGGTCA |
| | AGATGGCATCGTGAAGTGGTTGTTCACCGTAAACTGTAATACAATCCTGTTTATGGA |
| | TTTGTTTGCATATTTTTCCCTCCATAGGGAAACCTTTCTTCCATGGCTCAGGACACAC |
| | TCCTGGATCGAGCCAACAGGAGAACTTTCTGGTAAGCATTTGGCTAACTTTTTTTTTT |
| | TTGAGATGGAGTCTTGCTGTGTCGCCTAGGCTGGAGTGCAGTGGCGTGATCTTGGCT |
| | CACTGCAGCCTCCACTTCCCGGGTTCAATCAATTCTCCTACCTCAACTTCCTGAGTAG |
| | CTGGGATTACAGGCGCCCGCCACCACACCCGGCTCATTTTTGTACTTTTAGTAGAGA |
| | CACAGTTTTGCCATGTTGGCCAGGCTGGTCTTGAATTCCTCAGCTCAGGTGATCTGC |
| | CTGCCTTGGCCTCTCAAAGTGCTGGGATTACAGGCGTGAGCCACTGTGCCCGGCCTT |
| | GGCTAACTTTTCAAAATTAAAGATTTTGACTTGTTACAGTCATGTGACATTTTTTTCT |
| | TTCTGTTTGCTGAGTTTTTGATAATTTATATCTCTCAAAGTGGAGACTTTAAAAAAGA |
| | CTCATCCGTGTGCCGTGTTCACTGCCTGGTATCTTAGTGTGGACCGAAGCCTAAGGA |
| | CCCTGAAAACAGCTGCAGATGAAGATGGCAAGCACCCGCTGCAAGCTGGCCAGGTA |
| | CCTGGAGGACCTGGAGGATGTGGACTTGAAGAAATTTAAGATGCACTTAGAGGACT |
| | ATCCTCCCCAGAAGGGCTGCATCCCCCTCCCGAGGGGTCAGACAGAGAAGGCAGAC |
| | CATGTGGATCTAGCCACGCTAATGATCGACTTCAATGGGGAGGAGAAGGCGTGGGC |
| | CATGGCCGTGTGGATCTTCGCTGCGATCAACAGGAGAGACCTTTATGAGAAAGCAA |
| | AAAGAGATGAGCCGAAGTGGGGTGAGTGGAAGGAAGACTTTTAAAAAAAATTGTG |
| | GCCAAGTGCACATAGTGTACAATTTTCCATCTTTATATATATATATATATATATTTTT |
| | TTTTGAGACGGAGTTGCTCTTGTTGCCCAGGCTGGAGTGCAATGGTGAGATCTTGGC |
| | TCACTGCAACCTCCACCTCCCGGGTTCAAGCGATTTTCCTGCCTCAGCCTCCCAAGT |
| | AGCTGGGATTACAGGCATTGCACCTCCACGCCCGGCTAATTTTGTATTTTTAGTAGA |
| | GATGGGGTTTCTCCATGTTGGTCAGGTTGGTCTCAAACTCCCGACCTCAGGTGATCC |
| | ACCCGCCTCGGCCTCGCAAAGTGCTGGAATTACAGGTGTGAGCCACCGCGCCCGGC |
| | CGTAATAATTTTTAAATGTACAGTTCAGTAGTGTTAGGTACACTCACATTGTGCAAC |
| | CAGTCTCCAGAACTGTTTTCATCTTGCAAAACTGAAACTCCTCACCCATTGAATGAT |
| | AACTCCTCATTGCCTCATTCTCCCAGCCCTGGTAACCACCATTCTATTTGCCATCTCT |
| | GTGAATCTTACTACTCTGGGTACCTCATATAAGTGGAAACATACAGCATTTTTTCCTT |
| | TATGAGTAGTTTATTTCACTCAGCATAATGTCCTCCGGGTTCAACTATGTTGTAGCAT |
| | GTGTCGGAATTCCCTTCCTTTAAAAAAGACTGTATAAATACTCCATGGTACCATATA |
| | CCAAATTTTGTTTAGTTATCTGTTGATGAACACCTGGATTACTTCTACCTTTTTGTTA |
| | TTGTTAATGCTGTTATGAGTGTTGGTGCACAAATATCTCTTCAAGACCCTGCTTTGAA |
| | TTCTCTGGGGTATATACTCAGAAGTGAAATTTCTGAATCATACGGTAATTCTGTTAA |
| | ATATTTTGAAGAACTGCTCTACTGTTTTAGATACCATCTGCACCATTTCCCACCAACA |
| | GCACACAAGGGTTCCAATTTCTCCATATCCTAAGAACACGTTATTTTTATTTATTTTT |
| | TCTTTTTTATAGTAGCCATTCTAATGAGTGTGAGGTGGTATCTCACTGTAGTTTTGAT |
| | TTCCGTTTCCCTAATGATAAGTGATGCTGAGCACATTTTCACTGCTTGTTGGCCATTT |
| | ATAGAGCTTCTTCAGAGAAAGTTCTGTTCAAGTTCTTTTCTCATTTTCAGACCAGGTT |
| | TGGCTTTTGTTGTTGCCTTGTAGGAGTTCTTTATGTATTCTAGATATTAACCCCATAT |
| | CAGATATATGATTTACAAATATTTTCTCCTATTCTGTGGGTTGTCTTTTCATTTGGTT |
| | AACTGTGTCCTTTGATGCACAGAAGTTAGTATGATTTTTTTTTTTTTAAATCATGGCC |
| | GGGCATGGTGGCTCATGCCTGTAATTCCAGCACTTTGTGAGGCTGAGGTGGGTGGAT |
| | CACCTGAGGTCAGGAGTTCAACACCAGCCTGGCCAACATGGCAAAACCCCATCTCT |
| | ACTAAAAATACAAAAAAAATTAGCCGGGTGTGGTGGTGGGCACCTGTAATCCCAGC |
| | TACTCAGGAGGCTGAGGTAGGAGAATTGCTTGAACATGGGGAGTGGAGGTTGCAGT |
| | GAGCTGAGATTGCACCACGGCACTCCAGCCTGGGCTAAAGAGCAAGACCCTGTTTC |
| | AAAAAAAAATTTTTTTTTCTTGTATTGTCTTGGTTTTTTCTCTTTTCCCCTCTCACGGT |
| | AGTGCTATGAGAAGGTGGAGGCAAACTGTTTGCTTTCTCTAACTCCTTTAAAAAGCT |
| | GATGCAGACTGTACCTACCCCCTGGGCACCTGCCATGATTAGTCCCTCTTTATCTGA |
| | ATCTCAGCAGTGCCTAGACCTGCCTCCAATTCTCCATCCAGCTTCTCAGACCACATG |
| | TTTTGTCCAGTATCTGCTGTGTGGTCAGCAGGCTTGGGCCTCTGCTTCCTATGGCCCA |
| | GAGGGCAGCCTCTCTCCTTGAAGAACCGTGGAGTTTGGGGCAGAAGCCTTTCCTTTC |
| | ACCACCACTTTGGGAATTGGAATGTAAAAGAAATTCTCTTAATCCCCATGTACACAT |
| | TGGGGATAAGGTTCTTTCTTTTGTCTTGTGTTCTAGGCAAAGGTAGGGCTCAGAGAA |
| | GGGCAGGGTATTGCAGGATGATGATGTAGAGCTGGTGGTGATGGGAAGGAGGAGT |
| | ACAAATGAGTCAGTGGATAGGGAGACACATGTTAAGGCAGGGCAGCAGTGTGGGT |
| | GTAATGACACTGGGGAAACAGACAGCAGTGTGCAGCCGGGGGAGGTCAGAAACCA |
| | GTTGCTGCAATGGCTCTCACCTGTAGTCCCAGCTACTCAGGAGGCTAAGGCAGGAG |
| | GATCGCTTGAGCCCAGGAGCTCAAAACCAGCCTGAGCAACATAGGGAGACCTCATC |
| | TCTAAGTAAATAAAGCAATTATCATTGTGTCAGTACTTATATACTCACATACCCAAA |
| | TGAAAGTGTCATAATCACAACTGTAGAAGTACTTTGCTAAGCAGTTTTTATGGATTT |
| | TATGGCACATTCAAGAAACCCATGAAACAGCTTCTGGTATTATCCCTCCTGCAAGTG |
| | AATGAGGTGAGCCTGGAAGAGATCCTAACTGGCCCATGGTTTATTTCTAGCCATAAC |
| | CAGAGCTGGGGTAAAAAGCAGGGCATGTCTCTGATCAAAGCCTGGCTCTTAACCAC |
| | TACACCGTCTTTGCTTCCATATCTGGATTTTGAGGCAGAAAAGTTATAACTAGGGTT |
| | AGAAGTTTGTTTGTTTGTTTCCTGGGGTGGGGTTAGGTTTTAGGTAATAGACTTTGGC |
| | TAATTTACTGTCATGGCTCCTACCTGAGTCCCAGTTACTCAGGTGGCTGAAGTGGGT |
| | GCATCACTTGAGCTCAGGAGTTCCAGGCTGGAGTGAGCTATGACCATGCCACTGCCC |
| | TCCAGCTTTGGAAACAAAGCAAGACTCTGTCTCTTAAAAAAAAAGAATAAGATTTT |
| | GGTTTTGGCTTAGATTTTTTGGATTTTATTTTGTGATATGGTTACCAAGTAGACAGCT |
| | GTCAGAGGTCTTTTATGGGCTGGGCGCAGTGGCTTATGCCTGTAATCCCAGCACTTC |
| | AGGAGGCCAAGATGGGAGGATTGCTTGAGCCCAGGAGTTTGAGACCAGCCTGGGCA |
| | ACATAGTGAGAACCTGTCTCTACAAAAAATGCAAACATTAGTTAGGTGTGGTGGCA |
| | CGTGTCAATAGTCCCAGCTGTTCTGGAGGCTGAGGTGGGAGGATCACTTGAGCCCA |
| | GGAGGTTGAGGCTGCAGGGAGCCATGATTGTGTCATTGCACTCCAGCCTGGGCAAT |
| | AGAGTGAGGCCCTGTCTCAAAAAAAAAAAAAAAATCATTTATGTTACTTGTAGATT |
| | CCTGCCCACCTGAAGTTATCTGTCTTATATATTTCCCCTAGTGACTAACTCTTGTTTT |
| | ACCTAACCTGAATTTCTCCTATAGAATGCCTTCTTAAGATATGCTATCTAGTGACTGT |
| | TTGGATTTGTGATTCTCAACACAGATGCATGTTGGAATCACCTGGGAGCTTTTAGTA |
| | TGTGATGGTTCCTGGGCCTCACCTCAGAGGGACTGAAAAAGAACCTCTGGGCGTGC |
| | AGTCCTGAAGTCCATATTTTTAAAGCTTCCTGGGTGATTCTAAAGTCAGCCAGGATT |
| | CCCAACCACTAGATTAGGCACGAAATAGACTGGTCGTTCTCACTCTTGAAGAACCA |
| | GACATCTAGACTTGACAATAGAAGACGTGTGTAAAATGGATTGCAGGGAACTGCGG |
| | TCAGGGAAGGGTGACCTTTCTTAGCCCACAGCCGTGTTGGGGATATAGGAGAGCTT |
| | GGAGATGCCCTCACAATCAGGCTGCCAGGGGCAGGCAAAAGCAAACGAGGGTAAA |
| | AGAAACTTAGCCTGCTTCGCATGTTTGCTGATTGATTTCCAGGCCTGAGGAAGAGAT |
| | TCTGAGACTGGGGACAGTGGGAACACATGCTTGGCAGGTGGACAGCAGAAGTTGTT |
| | TTGAAACTAGGAGTGCAGAAATGCTCCCAACCAGACTTTTGACTGGATACCTGTCCT |
| | CTGCAACTCAGAGCCATGTTTTGATTTGGGGGATGTTTGGGGTCTCCTCTCTCATGCC |
| | AAATATTAGGCCAGGTTTCAATTGCATCCTCTGTGATAATAGTTCTGGGTTTTGACA |
| | CCTTTTTTTTCCCTTTTAGGTTCAGATAATGCACGTGTTTCGAATCCCACTGTGATAT |
| | GCCAGGAAGACAGCATTGAAGAGGAGTGGATGGGTTTACTGGAGTACCTTTCGAGA |
| | ATCTCTATTTGTAAAATGAAGAAAGGTAAGCGACTGGGGTGGTGCTTCTAGTTGAGT |
| | TTTAAGACCTGGTTCAGGAGGCTCTGGGAAGCTGAGCAGCTGGGTAACTTGGCCAT |
| | ACTATAGGTTCCTGCTCTTTGGAATGCAAAGGCCTGTCTCAGGAAATCCATTGTCAG |
| | TTGAAGAAACAAATTCATGTTTGTGTGGCTCTTGCAGTCCCAGAGCCAGCGGCGGTG |
| | ATTAAGCGTTTCAAGTTTGATAAGGCATAGGATGCAGATTACTTAAAGGAATAAGA |
| | TTGATTCAAATGATCTTTGGGCTGGTAGCTACACATAGAGATGGGCCTTCTGCTCAG |
| | CTGTGTATCTTAAAAACTTGACACCAGAGATTCTCGGCACCTTTCCTACCCAGTGGG |
| | GAGGAGCTGCACCTTCCATTTCTCCATTCCGGTTACCACTCGCTTCCGATGACAGGC |
| | CCCAGCTGAGAGCGCATCTCAGGTGGATGTGTGTATACTTTCCCCCTAACTTCCTGT |
| | CTTTGCCGTAGATTACCGTAAGAAGTACAGAAAGTACGTGAGAAGCAGATTCCAGT |
| | GCATTGAAGACAGGAATGCCCGTCTGGGTGAGAGTGTGAGCCTCAACAAACGCTAC |
| | ACACGACTGCGTCTCATCAAGGAGCACCGGAGCCAGCAGGAGAGGGAGCAGGAGC |
| | TTCTGGCCATCGGCAAGACCAAGACGTGTGAGAGCCCCGTGAGTCCCATTAAGATG |
| | GAGTTGCTGTTTGACCCCGATGATGAGCATTCTGAGCCTGTGCACACCGTGGTGTTC |
| | CAGGGGGCGGCAGGGATTGGGAAAACAATCCTGGCCAGGAAGATGATGTTGGACT |
| | GGGCGTCGGGGACACTCTACCAAGACAGGTTTGACTATCTGTTCTATATCCACTGTC |
| | GGGAGGTGAGCCTTGTGACACAGAGGAGCCTGGGGGACCTGATCATGAGCTGCTGC |
| | CCCGACCCAAACCCACCCATCCACAAGATCGTGAGAAAACCCTCCAGAATCCTCTTC |
| | CTCATGGACGGCTTCGATGAGCTGCAAGGTGCCTTTGACGAGCACATAGGACCGCT |
| | CTGCACTGACTGGCAGAAGGCCGAGCGGGGAGACATTCTCCTGAGCAGCCTCATCA |
| | GAAAGAAGCTGCTTCCCGAGGCCTCTCTGCTCATCACCACGAGACCTGTGGCCCTGG |
| | AGAAACTGCAGCACTTGCTGGACCATCCTCGGCATGTGGAGATCCTGGGTTTCTCCG |
| | AGGCCAAAAGGAAAGAGTACTTCTTCAAGTACTTCTCTGATGAGGCCCAAGCCAGG |
| | GCAGCCTTCAGTCTGATTCAGGAGAACGAGGTCCTCTTCACCATGTGCTTCATCCCC |
| | CTGGTCTGCTGGATCGTGTGCACTGGACTGAAACAGCAGATGGAGAGTGGCAAGAG |
| | CCTTGCCCAGACATCCAAGACCACCACCGCGGTGTACGTCTTCTTCCTTTCCAGTTTG |
| | CTGCAGCCCCGGGGAGGGAGCCAGGAGCACGGCCTCTGCGCCCACCTCTGGGGGCT |
| | CTGCTCTTTGGCTGCAGATGGAATCTGGAACCAGAAAATCCTGTTTGAGGAGTCCGA |
| | CCTCAGGAATCATGGACTGCAGAAGGCGGATGTGTCTGCTTTCCTGAGGATGAACCT |
| | GTTCCAAAAGGAAGTGGACTGCGAGAAGTTCTACAGCTTCATCCACATGACTTTCCA |
| | GGAGTTCTTTGCCGCCATGTACTACCTGCTGGAAGAGGAAAAGGAAGGAAGGACGA |
| | ACGTTCCAGGGAGTCGTTTGAAGCTTCCCAGCCGAGACGTGACAGTCCTTCTGGAAA |
| | ACTATGGCAAATTCGAAAAGGGGTATTTGATTTTTGTTGTACGTTTCCTCTTTGGCCT |
| | GGTAAACCAGGAGAGGACCTCCTACTTGGAGAAGAAATTAAGTTGCAAGATCTCTC |
| | AGCAAATCAGGCTGGAGCTGCTGAAATGGATTGAAGTGAAAGCCAAAGCTAAAAA |
| | GCTGCAGATCCAGCCCAGCCAGCTGGAATTGTTCTACTGTTTGTACGAGATGCAGGA |
| | GGAGGACTTCGTGCAAAGGGCCATGGACTATTTCCCCAAGATTGAGATCAATCTCTC |
| | CACCAGAATGGACCACATGGTTTCTTCCTTTTGCATTGAGAACTGTCATCGGGTGGA |
| | GTCACTGTCCCTGGGGTTTCTCCATAACATGCCCAAGGAGGAAGAGGAGGAGGAAA |
| | AGGAAGGCCGACACCTTGATATGGTGCAGTGTGTCCTCCCAAGCTCCTCTCATGCTG |
| | CCTGTTCTCATGGGTAAGGAAACTCGGCTTCCAGGTGCTTCCTCCTGCTTCCTCGCCA |
| | GCTTCTTCTTGGCGCTTGCCTCCTCTCATCTCTTTTCAACTATCTTCCAAATACTGTTG |
| | CCACAGCTACATCATAATGCCACCACTGTCTGTTTGAGACTCCTTCATGAGCAAAGA |
| | TTGATGTATGGTAGGTGGATAAATGGGATGAGGAAAAAAAAAATAAAACAAGGAA |
| | CAAATGTTTGGGGAATGCCAGTTTAGCACAAGGTATTAAGTAGGATGTAGGATTGC |
| | CTACAAATATTTGTCAACTGAAATTTCTTGTAAGCTACTTGGAGCACTAGTGCCTAA |
| | GAGTCAGTCAATGTCTGTGGGGCAGAACAATGCCAGAGAGTCTGTGTCCCAGGAGC |
| | GAGGGGACAGAGGGCCTCAGAGCTGGAGGCTGGGGGGATGTTTCTTAGAAATGTGC |
| | AATCTAAGCCTCCTTTAAGGAATGGTGGGACCTGGGCGCATAGAGAAGAAATTAAA |
| | GGAATTCTGCACTGAAGACTGGTATGAGCCAAATCATGAATGCAGTCTGAGAAAAA |
| | GTGTGTGCAGGTGACAGTGGAGAGAGACACAGTTTCCTTGGAAAAAAACATGATGA |
| | AGGTAAATCAAGGGAAATAGAGCAGAAATGTGGAGACCCTCTTTCTAGGCCCCCAT |
| | GTTGCTCTGCACTGGGCATTGCCAAAACCCCGTTTTCGGGTCTGTTCACACACTTGCT |
| | GGGTGGTTGGAAGCTAGTGGCCAAGTCTGTGCAATGAAGTGGTTGAGTCACCTGAC |
| | TTCTGAGTCCCTTTTGGGTTTATCGTTCAGTGCCTCCTGCAGAAGGACCACCCACAG |
| | TGGTTGTAGGAAGGTGGCCACCCTGTGATGTGTGAGTTTAGCCTGGATAGAAGACA |
| | CTTGACGTGGGGCAAAATTAAGGGAAACAGGAGGCTTGGCCCTTCCACAGGCAACA |
| | ATGAGTTTCAGCTTTCTTGATCCGTTGACATTACCATGACCAGGACCACCTTCCTAGT |
| | CATGGTTTGGCCATGAAGGACTGACTGCCCACAGGGCCGCAGTGGTGGTGAAGACC |
| | CAGGTCCTGGTCACAGGGACTTGAGGTCTGGCTGTGGGACAGTCCGGAAACAAGTG |
| | CAGATGTCGGGGAGGGGGTCATGGGCTCTGACAAGGAAACTTGCAGAGTATCCAAA |
| | GCACAAGGGGACGAACCTGTTCACCAGCGGGGCACGGGTGGGTGTCCCCGCTCAGC |
| | CAGCTTCCGGAGGATGGGCAGTGAGAGAGTGAGAGAAGGAAAGGGGCAGCCAGGC |
| | TGGGGGCTTACGGTGTTGAGAGAGAGGGCAGGTGTGGGAAAGCAGGGTACGGTTTC |
| | TACGGCAGCAAACAGTTCCGAGTGACTGGAAAAAAGACAGTTGTCTTCATTCATCA |
| | GGTTGCCATAACAAAACACTGCAGGCTGGTTGGCTTATAAGCAAAAGGAAGGTTCA |
| | CCTTACGGTTCTGGAGGCTGGGAAGTCCATGATCCAGGCTCTGGCAGATCTGGTGTT |
| | TGGTGAGGGCCTGTTCCCTGGTCGATGGTGCCTTCTCACTGTGTCCTTACATGGTGA |
| | AGAGGCAAGGCAGCTCTCTGGGGTCCCTTTCCTAAGGACAGCAATCCCACTCCTGA |
| | GGGCTCCACCCTCATAACTTAACCACACCTCAAAGGCCCCATCGCCTGATACGTCAT |
| | GTTGAGGGTTAGGATTTCAGCGTATGGATTTTGGGGCACACAAACATTCAGACCATA |
| | GAAATGGGAGAAGAAGAGTAAGACAGAGAGAAAAGTAGGCAGAACTACTCATTAA |
| | GAGTGAGGTGGAAGGTGGAGAGGGCAGGGAGACCTTATGAAGATCACACATCTGT |
| | GAGGAAGGTTTGCACACTCTCACTGAAGCCCCGGTAGGAGGCTCAGCACCCATTTCT |
| | CTAGATAGCACCTTCCTTCCTCTGAGGACAGATTCTGACTGGAGCCCTGGTAGGGGG |
| | CTCAGCACCCATCCCTCTAGATGGCACCTGCCTTACTCTGAGGACAGACTCTCACTG |
| | AAGCCCCGGTAGGAGGCTCAGCACCCATTTCTCTAGATAGCACCTTCCTTCCTCTGA |
| | GGACAGACTCTGATTGGAGCCCTGGCAGGGGGCTCAGCACCCATTTCTCTAGATAG |
| | CACCTTCCTTCCTCTGAGGACAGACTCTCACTGAAGCCCCGGTAGGAGGCTCAGCAC |
| | CCATTTCTCTAGATAGCACCTTCCTTCCTCTGAGGACAGACTCTGATTGGAGCCCTG |
| | GCAGGGGGCTCAGCACCCATCCCTCTAGATGGCACCTGCCTTACTCTGAGGACAGA |
| | CTCTCACTGAAGCCCCGGTAGGAGGCTCAGCAACCATTTCTCTAGATAGCACCTTCC |
| | TTCCTCTGAGGACAGACTCTGATTGGAGCCCTGGCAGGGGGCTCAGCACCCATTTCT |
| | CTAGATAGCACCTTCCTTCCTCTGAGGACAGACTCTCACTGAAGCCCCGGTAGGAGG |
| | CTCAGCACCCATTTCTCTAGATAGCACCTTCCTTCCTCAGGACAGACTCTGACTGGA |
| | ACCCTGGTAGGGGGCTCAGCACCCATCCCTCTAGATGGCACCTGCCTTACTCTGAGG |
| | ACAGACTCTGACGGGAGCCCTGGTAGGGGGCTCAGCACCCATCCCTCTAGATGGCA |
| | CCTGCCTTACTCTGAGGACAGACTCTGACTGGAGCCCTGGTAGGGGGCTCAGCACCC |
| | ATCTTTCTAGATGGCAGCTGCCTTACTCTGAGGGCAGGAGCTTGCTTTGTATGTTCC |
| | AGTCCCTTCTGAGAACCAGAAGCCTGAGCATCTATTAGAAATGCAGGATTGTGGAC |
| | CCAATCTTAGACCTAGAGAGCTAGACTTTGCATTTGAATGAGAGCTACAGGTGATTC |
| | AAAGGTGACTGCATGTGTGGGTGCAGTGGCTCTTGCCTGTAATCCTAGGTTTTGGGA |
| | GGCCAAGATGGGGGGATTGCTTGAAGCCAGGAATTCAAGTCCTACCTGAGCAGCAA |
| | AGTGAGAGCACCTCTCTACAGAAAAACAAAAGCAAAAATGTGACTGCATGTTGGAG |
| | CCACCTGGCTTTTTTTAAATAAAAAAGATCCATATAGCTGGACACAGTGACTCGTGC |
| | CTGTAACCTCAGCTACTCGGGAGGCTGAGGCAAGAGGATTGCTTGAGCCCAGGAGT |
| | TTGAGGCTGCAGTGAGCTATGATCATGGCACTGCATTCCAGCTTGGGTTTCATCTGA |
| | AAAAAAATTTAAAAAATTCATCTAGTTCCACCTCAGAGCTTCTGATTTTATTTCCAT |
| | GGATGTGGCCTGGGCTTGCGATTTTCTTTTTCTTTTTTTTTTTTTTGAGATGAAGTCTT |
| | GCTCTGTTGCCCAGGCTGGAGTGCTGTGGCATGATCTTGGCTCACTGCAACTTCCAC |
| | CTCCCAGGTTCAAGTGATTCTCCTGCCTCAGCCTCCCTAATAGCTGGGATGACAGGC |
| | TCGTGCCACCACACCCAATTAATTTTTGTATTTTTAGTAGAGAGAGGGTTTCACCAT |
| | GTTGGCCAGGCTGATCTTGAACTCCTGACCTCAGGTGATCTGCCCGCCTCGGCCTCC |
| | CAAAGTGCTGGGATTACAGGTGTGAGCCACCATGCCCAGCCTGGCCTGAGGATTTTT |
| | GTGAGCACTCCTGATGCTGTTGTGTAGCCAGGAGTGAGGACTGCCCTCTGATCACCC |
| | AGCACAGAACGTTCAGGGCCATGTTGGGACAGTGGACTGGTAAGGGCGAGAGGAG |
| | GACACTGGGGAGCAGGCTGTAGGGTGCCACAGTCCGCCACAGTCACAGGCTCATGC |
| | TCTCCGTCCGAGAGGAGGCAGAACTGCTTTTACCAAGGCTCATCCAGCCCTCTTCAT |
| | TTCTCTGGGAACAATTTTTGGTTTCGTGACCCATTTCTTAATCCCCGGAAGGCATTTC |
| | TCTGAACTGGTGCCAGGCACCCCGGCCCCCAGCTCCAGTTAGTTATTATTCGAGGCT |
| | GATTTCTTTTCTGTCTGTCTTCCTTCTAATTCCTAGATTGGTGAACAGCCACCTCACT |
| | TCCAGTTTTTGCCGGGGCCTCTTTTCAGTTCTGAGCACCAGCCAGAGTCTAACTGAA |
| | TTGGACCTCAGTGACAATTCTCTGGGGGACCCAGGGATGAGAGTGTTGTGTGAAAC |
| | GCTCCAGCATCCTGGCTGTAACATTCGGAGATTGTGGTGAGTCCCCGTGCATGTGAT |
| | CTGTGTGAGTGCAAGTTCATATGAGAGAGAGAGAGAGAAACAGACTGGAGAAAGA |
| | TCTTCAGGACCAGGTTGCTGGTGTGGTTTCTTTCTTTTCTTTTTCTTTTTTTCTTTTTTT |
| | GAGGCAGAGTTTTGCTCTTATTGCCCAGGCTGGAGTGCAATGCCGTGACCTCGGCTC |
| | ACTGCAATCTCTGCCTCCTGGGTTCAAGTGATTCTCCTGCCTCAGCCTCCTGAGTAGC |
| | TGGGATTACAGGCACCCACCACCATACCCAGCTAATTTTTTGTATTTTTAGTAGAGA |
| | TGGGGTTTCATCATGTTGTCCAGGCTAGTCTCGAAGTCCTGACCTCAGGTGATCCGC |
| | CCGCCTCGGCCTCCCAAAATGCTGGGATTACAGGCATGAGCCACCGCACCCGGCCG |
| | GTGGTGTGGTTTCTGCTGTAACAAAGTGCCACAGACTGAATGGCTTCAACAACAAA |
| | AATTCATTTTCTTGCAGTTCTGAGGCCGAAAGTCTAAGATCAAGGTGTCAGCAGGGT |
| | TGGTTTCTTCTGAGGCCGCCCTCCTTGGTTTGTAGATGCCCCCTTCTCCCTGTGTACT |
| | GATGCCATCTTGCCTTGGGGGGAAGAGTGGCCTAGTCTCCTCTTCTTACAGGGACAG |
| | CAGTCCTGATGGATTAGGCACAGTCAGGCATGACTACACAAGACCTAATGTTAATCT |
| | AGTCACTCCTTTAAAGACCCCATTTCCCAAAACAGCCACATTATGAGGTCTTGAGGA |
| | TTAGGGCTTCAACATGTAAATTTTTGGGGGGGCACAGTTCAGCCCAGAACAAGTGA |
| | GAAGGTAGAATGGGTTGGAGGCCAGCTGGGGAAGTTTTGTGTGGGGAAGAATGAAT |
| | GAGGAACTAAAAAAAAAGTCATTTCTTGAAACAACCATGATGCTCGATATATTCCA |
| | TTTCCTCATGGGCCATGTTCATAGATGAGAGTTGGAGAATTTGCCATTTACAACAAT |
| | GGCAACCTCACCTAAAAACTATGAAAATACTTTTTTTTTTTTTCTTTGAGACAGGACC |
| | TCTCTCTGTCACCCAGGCTGGAGTGTGTGATCACAGCTTACTGCAGCCTCGACCTCC |
| | CAGGCTCAAGCAATCCTCCCACCTCAGCTCCCCCGAGTAGCTGGGACTACAGGCAT |
| | GCACCACCATGCTGGCTAATTTTTGTATTTTTTTTGTAGAGATGTGGTCTCATCACGT |
| | TGCCCAGCCTGGTCAGAATGCTTTGTATGGGTTGAAATGTCACAGGGAATCTCCTGT |
| | GACTTTATTAATAATGTGCTTGCTCACACCTGTAGTCCCAGCTACTCGGGAGGCTGA |
| | GGCAGGAGAACTGTGTGTACCCGGGAGGCGGAGATGGCAGTGAGCCGAGATCGTG |
| | CCATTGCACTCCAGCCTGGGTGACAGAGCGAGATTCCATTTCAAAATAATAATAATA |
| | ATAATAATAATAAAAAATGTGCTTGCTTTCTCCCACACCATGAATCAGATTCACACG |
| | CCCCACCGCGCTGGTGCCTGGGCCGCCTCACTAGCTCTGTTTCATTCCCTGCTCTGTG |
| | GACGCATGCAGGCGTCTGCATTTTTGAGGAGCTGCACAGCAAGTGTTAAAGTCCGC |
| | CACTCTAGGACATTTTGCTACCTCCCTCTCCGGCTCAACACACTCACCTTGTCACCAG |
| | TCTTATCAATTTTTCCTCCTACCCGAGCTGTTTTCCACTTTTCCTCGTCCTCACCCCAC |
| | CAGCCCAGGTCCAGCAACTGTCACCACTTGCCTGGACACGCAGTGACCTCCTAGACC |
| | ATCAGCTCCCCGGGGATGGGGTTTGCGTTTGTTTGTCTTGCTGTCATCTTTGCCACGC |
| | CTGGACAGACATCTTAGTACCCATCAGTGGTCAAAAGCACGCACTGAATGAATGTT |
| | GGCTCTTTGGTCTCCCTGTGTCTGCCTTTTTCCCTCACCCGACCATTCTCTGCACAAA |
| | AGTAAGATGGTCCTTTAGAAAATTCTGATTCGATTGTGCTGTTCGGTGGCTTAAATG |
| | CTTTCTGTCTGCTCTGCTCTTTATTTTTTACTTTATGTATTTATTTTTGTAGATGTAGG |
| | GGGCGCAAGAGCAGCTTTGTTTCATGGATATGTTACTGCACAGTGGTAGTGTCTGGG |
| | CTTTTACTGTAACTACCCCTTTTTTTTTGAGATGGACTCTCGCCGTGTTGCCCAGGCT |
| | GGAGTACAGTGGAGTGATCTCGGCTCACTGCAACATCCACCTCCTGGGTTCAAGCA |
| | ATTCTTCGAGTAGTTGGGATTACAGGCATGGGCCACCACGCCCAGCTGATTTTTGTA |
| | TTTTTAGTAGGGACAAGGTTTCACCGTATTGGCTAGGCTGGTCTCAAACTCCTGACC |
| | TCGTGATCCACCCGCCTCGGCCTCCCAAAGTGCTGGGATTACAGGCGTGAGCCACCG |
| | TGCCCGGCCTTAGTGTAACTCTTACCCAAATAGTGAACATTGTACCCAATAAATAAT |
| | TTTCCAGCCCTCACCTGGCTCCCACCCTCCCACCCTTCTGAGGCTCTAGTGTCTATCA |
| | TTCCATGCTCTATGGATGTTACCCTTTGTTTAGCTCCCACTTGTAAGTGAGAACGTGT |
| | GGTATTTGACTCTGTTTCTGAGTTGTTTCACTTAAGGTAATGGCCTCCAGTTCCATCT |
| | GTGTGGCTTCAAAGATGTGACTTCATTCTTTTCTATGGCTGAGCAGTATTCCATTATG |
| | TATATAGATCACACTCTTCATCTTTGCTCTTTTGGTAAATTTCAAATCGTAGTGCCAT |
| | GGAGACTGGTTGTTTGGGACATTTAACCTTATTTCTCCAACTTCATGCCCAAATTCTC |
| | TCCCCACTGCCATTGCCCCTGTATGATTCTGGAGAGGCAGACACAAACAGGGAAGG |
| | GTAGGGGACAGTGAGATGCTTCGCAGAGAAGTAGAGATCCATTTGTCCATTTGCTTG |
| | AAGTTGGTGTAAGGATAGGGACAGAAAGGAAGGTGGTCTAACAGGCTCCAGGACCT |
| | ATGAACTATTGCCCTGGAAAGAAGCTAGAATGTTGTTCTTGCCCCCATGACAGGGAT |
| | AAAGTATAGTTTTTGTTGCTGGTGGTGGTGGTGACGGTGGTAGTGATGTGCGTGTGT |
| | GTTTGTGTGCATGTGTGTGACGGGGAAACTAATCAAGAATCAGAGCTGCAGCGGGA |
| | TGTGGAATCCCAGCACTTTGAGAGGCTGAGGTGGGCAGATCACCTGAGCCCAGGAG |
| | TTCAAAACCAGCCTGAGCAAGCTGGTGAGACCTTGTCTTTAGAAGATAATAATAAT |
| | AATAAAAATAAAATATAAATAAAATAAAATGGCTAAGCATGGTAGCACATGCCTGT |
| | ATTTGCAGCCACTTGGGAGGCTGAGGCAGGTGGATCCCTTGAGCCCAGGAGTTCGA |
| | GGTGGCAGTGAGCTATGATCTCGCCAGTGCACTCCAGCCTGTGCAACAAAGTGAGA |
| | CCCTGTCTTTAAAAAAAGAAAAGAAAAGAAAAGAAAAAGAATCAGAGCTGCTTTTC |
| | CTTTCAATATAGATGGAGGGCTGGGATGAAATCGTCAAACACAATGGGGTGACAAT |
| | GTTGGTGAAGACAAGAAATAGCTGAGTGCAGCACTGGCGAGGGCAGTGCCGACTGC |
| | GGGCACGTCAATGGTTAGGAACACGAGGGAAGGAGGTGGGTGCACAGGCCCACAC |
| | CGCCCGCTTCACTTACAAACCAGGAGCGCACCATTCACATGTCTGGGGGGTGATCTC |
| | AATGCACCCGGCACTGTTTACGGCCGGGAGTTCACTGACAGGTGATCCCTCTGCTGT |
| | GACTGGGGAGACTGGGGGCTGACCTCCCAATGTGCCTTGTGCTGTATTCCGGAGCTC |
| | TCTGGTCAGGTGTATTCTGATGCTTTCTCTATTCCAGAGCTCTCTGGTCAGGTGTGTC |
| | CTGATGCTTTCTCTATTCCAGAGCTCTCTGGTCAGGTGTGTCCTGATGCTTTCTCTAT |
| | TCCGGAGCTCTCTGGTCAGGTGTGTCCTGATGCTTTCTCTATTCCGGAGCTCTCTGGT |
| | CAGGTGTGTCCTGATGCATTCTCTATTCCAGAGCTCTCTGGTCAGGTGTGTCCTGATG |
| | CTTTCTCTATTCCAGAGCTCTCTTGTCAGGTGTGTCCTGATGCTTTCTCTATTCTGGA |
| | GCTCTCTGGTCAGATGTGTTCTGATGCTTTCTCTATTCCGGAGCTCTCTGGTCAGGTG |
| | TGTTCTGATGCTTTCTGTATTCCGGAGCTCTCTGATCAGATGTGTTCTGATGCTTTCT |
| | CTATTCCGGAGCTTCCTGATCAGGTGTGTCCTGATGCTTCCTCTGTTCTGGAGCTCTC |
| | TGGTCAGGTGTGTTCTGATGCTTTCTGCCTCTGTTCTTGGCATGAAGGTTGGGGCGCT |
| | GTGGCCTCTCGCATGAGTGCTGCTTCGACATCTCCTTGGTCCTCAGCAGCAACCAGA |
| | AGCTGGTGGAGCTGGACCTGAGTGACAACGCCCTCGGTGACTTCGGAATCAGACTT |
| | CTGTGTGTGGGACTGAAGCACCTGTTGTGCAATCTGAAGAAGCTCTGGTGAGTCGA |
| | GCCCGTTCCCCTAAGGAAGTTCTGCCAGCGAGGCGTGCTGCGCACTCTGGCTTCAGT |
| | GAGGCCCGGTGGGCTGGGGTTTGAGTGAGAATGGCCTTGGCGGCTCGGGTGACTGC |
| | GTGTGCTTGTCTTGGGGTGTCTAGCATTGCGGTCAGTGAGTGTTTGTCGTGGGGAAA |
| | GCCACACACTCAACCCTGACAAGGAGCATAAATAATAACAAAAAGAAGATGTCTGC |
| | CTTTAGGGTGCCTACTGCCTAGCTGACAAAAAGAAAAAATCAGTGTAATGCATTGCT |
| | TTAGAGGAGTTTTAGAACTTCATGACAAGCCCTGGGTCCTCATATTGTATAAGAAAA |
| | ATGGTGCTAAAGCAATGCAGACCCTCGATGTTGTATGCAGCCCTTCCCTGAGTGCTT |
| | CTGGGAGACTTGCATTTTGAAGGAGAGAAAAAACCACTTAAAAATGGAAGGGCCAT |
| | TTCTCACCCATTAGGATGGCTATTATCAAACAAAAGCAAGAAAACTAGAAAACAAC |
| | AAGTATTGGTGTGGGTGCGGAGAAATGGAAGCCATTGTGTGTTGCTGGTGGGAATG |
| | TAAATGATGCAGCCACTATGGAAAACAGCACAGCAATTCCTAAAAAATAAATACAG |
| | AATTACTACTGGAGCCAGCATTTCCACTGCTGGGTATATCTGCAAAAGAGTTCAAAG |
| | CAGGAACTCAGCCAGGAGCGGTGGCTCACACCTGTAATCCCAGCATTTTGGGAGGC |
| | TGAGGCAGGTGGATCACTGGAGGTCGGGAGTTCGAGACTAGCCTGGCCATCATAGT |
| | AAAACCTTGTCTCTACTAAAAATACAAAAATTAGCCAGGCATGGTGGTGCATGCCT |
| | GTAATCCCAGTTACTCGGGGTGGAGGCAAGAGAATCTTTTGAACCTGGGAGGCGGG |
| | GGTTGCAGTGAGTCAAGATCCCGTCACTGTACTCCCGCCTGGGTGACAGAGGAAGC |
| | ATTAAAAAAAACAGAAGGCAGGAACTCAAACATGTTATTTGTACATCTATGTCCAT |
| | AGCAACACTATTAGAAATAGCCAAAAGGTGGAAATAACCAAACGTCCTTCAACGGA |
| | TGAAGGGTGTTGAAAAGTGCTATATATGTACATTCAGCTTTAAAAAGAATTCTAGTA |
| | CACACTACAACATGGATGAGCCTCGGGGACATTATGCTAAGTAAGATAAGCTAGAC |
| | TCGAAAGGGCAAATACTCTGATTGTCCTTATAGGAGGTGTCTAGAACAGACAGATT |
| | CATAGAGACAGGAAGAAACGTGGTTGCAGGCAATGGGAGGGCGGAGAGTGGGAAG |
| | ACATTGTTTAATGGTTATGGAGTTTCAGTTCTGCAAGATGAAAGGAGTTCTGGAGAT |
| | GGTGGTGGTGATGGTTGCACAACAATGTGAAGTGCTTAAGGCCATTAATTGTGCACT |
| | TAAAAATGGTTAAAATGGTAAATTTTATTTTACACGTATTTTACCACAATAAAGCTG |
| | AATGTAGGGAGCTGGGAAGATGTAGTATTGGTGGGAGCTTGGAGAATGCTTACTTG |
| | TTATTCCACCAAATGGAAAAGGAAATCAGAAGTGTACATAGAGCTTGTGTCCACTC |
| | CCTTTCCATGTGTAAACTGGAGTAGAGGCAGTGGCAGGTACGGGTGCTTCCTTGTCC |
| | ATGGTGGAGCGTGGGTGAGAGACATGACTGACATTCTGCCATCTCTATGGAAGGTT |
| | GGTCAGCTGCTGCCTCACATCAGCATGTTGTCAGGATCTTGCATCAGTATTGAGCAC |
| | CAGCCATTCCCTGACCAGACTCTATGTGGGGGAGAATGCCTTGGGAGACTCAGGAG |
| | TCGCAATTTTATGTGAAAAAGCCAAGAATCCACAGTGTAACCTGCAGAAACTGGGG |
| | TAAGTCTTCATGGGTGTCTTACCAGAAAAGTAACTTCTTTTCAGAGGTGAATTATTT |
| | GGAAAATGTGGATGGGGGTTACTTTGGTCAGGCAGAGCTGAAGGTAGAGTAAGGAG |
| | GTAAAAACATCAGAAATGCTTTGAAGGACTGGAGGCATATAATGTATACTGCTGCT |
| | AATAAGATAATTTATGCTAATTTATGATTATAGAACTAGAGTTTCTTAGTGTTGGAA |
| | GAAACCTTAGAGATGGTGCAGCCCAATTGCCTGTTGAGGGAAAAACTTGCTTCCAA |
| | GACACTCACATGAATAGTCACCCATTTTATGTGTGCATGCTTGCAAGCATGCAAGTT |
| | ATCGCATAGTGCATGCAGCCTCTACACTAGGAAAGAGCTCTCACCCTGCAGCCTCTA |
| | CACTAGGAGAGGACTCTTACACTGCAGCCTCTATATCCTCAACAGTGTCATGGCTCA |
| | GCACCTTCACCTATGTAGCCATGCTCTCCCAGGTACACAGGCTGGTTTGTTAGTGTC |
| | TGTCTTTGAAATGTGGTGCCCAGACCTGATGATGCATGGTGGATGTTTTACCTCCTG |
| | TTGCAGAGAACTATGGTTTCACTGGTGGAATCTCTGATTTCCTTGTTTTTTTTGGCAG |
| | TTACATAAGCCTTTTGATTCATGTTGGAATTCTGCAGTAAACAGTAGTAGGGAATAC |
| | CCACTAATAATCATGCACTTTCCCTGTACCACTTTCCTGTATCACTTTCCCTGTATCA |
| | CCTGCTCTTTCATGTTCTTACATGGATTTTCTCAGTTGGCTTCAGAAAGATGCTATAG |
| | GGTAGGGTATCTGTGTCTCCAATTTAAAGAGGAGAAAATAAAATTCAGGGAGCCTC |
| | ATAACTTTGTCCAAGGACACTCAGAGAGAAGGTAGTGGAACTCAGTGTAGACAAAC |
| | GTCTGTGACCTCCCAAGCCACTGCCTTAATTCCCAGTGCATTGCCTTAGGAGATGTT |
| | GCTAATGGGCTCTCTACAGGGCTTCTTCATCTGGCTCTCAGCTTAAGGGTGGGACCT |
| | CCAATTTAGCTTTGCTAAACCTCATCTTACCTGTCTTTTGACCCATTGACTAGGCAGT |
| | TTTGAATCTTCATTCTGACAATAATATCTACAAATACAATAAGCAAATTGGATAGGT |
| | CTTCATTCATTCACATTATTAATATATATACCAGCCAGATCAGCATTCAAGACAAAA |
| | TTTGGCAGGAAAGGTCTTCCCAGAGGAGTAATCCACTAATCCATAATTAACAACCA |
| | ACCTCTGTTCACAAAGCTCTGATACTTCCTTGGACCTCTCCCATCAGCCAATCCTCAG |
| | ATGTGACCATATCTCTTTCTATGAAGTCTTCCTGCAGTGCCTTCATTTCCTCGCCTTA |
| | GGGTATTACCCTTCCTTAGCAAATCGCCTTAGAATATTACTCTTCCTTGGCAATTTGA |
| | CTTCTGCTTCTCTAAGTTTGTTCAACAGACATGATTTTGTCCCTTCATTTTGCAGGCC |
| | CTCTGGGGGCTCTGTGGTGGGCACTGACAGCAGCAGTGAGTAAAACAGACCAAAAT |
| | CCTGTTTTCACAACCGTTCTTTTAGGGAAGTGATAACAGATAATAGACATGCTAAAC |
| | AATATACCCTGTGTATAAGAGCTATAAGAAAAACTGGAGTGACGGCGAGGGGTGCT |
| | TCTCTGCTCACGTTTGGCGAGTGCCTCTCCGAGCGGGTGACATTGGAGCAGGTGCTC |
| | AGTGAAGCGTGGGATGGGCTGTGAAGGAGCCGGAAGAGCCCTCCAGGCAAAGCAG |
| | ATGGAACCGAAGAGGCCCAGGCGTAGGCACTCAGGTGACTCTCAGTGATCCTGGTT |
| | CCAAATTCCACATGACAAACAAGCCAATTACAAATGTGCCTGATGCATCTGTAATAT |
| | GCTCTATGTAGTTACTATTGTCAATACCTAACCTGGTAGAGCTGCTGTAATAAAGTA |
| | TCACAAACAGAGCGGCCTAAACAACAAGAGTTTATTGCCTCTCAGTTCAGGAGGCC |
| | AAAGTCTGAGACTGAGGCATCAGCAGGCTGGGTCCTTATAGGTCGTACAGGTCCTTC |
| | CCAGTCCTTACTTTAGTTGACATCTGCTGCCTTTAATTCATTTCACTCTTCCTCTTTGG |
| | AAGCCTTCTTCGCTCTTATTTTTAGGTCACTACTTAGTCTTTCCTGCTAATGTTATAG |
| | CTTCCCCCTCCCTGGATCCTGCATTGGCTTCTCTTCCTGTGGTCTGGGGATTTCACGA |
| | CTGGTTTAGTTGTGTTTTTTTTTTTTTTTTTTTTTTTGAGTTGGAGTCTTACTCTGTCAC |
| | CCAGGCTAAAATGCAGTGGCGCAATCTTGGCTCACTGCAACCTCTGCCTCCCGGGTT |
| | CCAACAATTCTTCTGCCTCAGCCTTCTGAGTAGCAGGGACTACAGGTGTGCACTGCC |
| | ATGCCTGGCTAATTTTTGTATTTTTAGTAGATACGGGGTTTCACCATGTTTGCCAGGC |
| | TGGTCTCGAATTTCTGACCTCAGGTGATCTGCCTGCCTTGGCCTCCCAAAGTGTTGG |
| | GATTACAGGCGTGAGCCACACACCCGCCTGGTTTACCTTTTTTGTTGTTGTTGTTACT |
| | AAATATTTCTAAACTCCCTGAGAATTCTCATATACCTCTCAATTTCAACAACAGTAG |
| | GTCTCAAACCCAGATATCATTCACAAACTTTTTATTAGAAAAGTAACACATGCTTGG |
| | TGTAAACATATCATAGTACAGACAAAGTGGAAAGTGAAAGCTCCTTCTCAGCCCTCT |
| | TCTGAGAGAGCCATTTACAATAGTCTGATGAACAACCATCCAGACTTTTTTTAATAC |
| | AATAGATTGTTATATAATCCATTCATCTGTCCATCCATCCATCCATCCATCATCCATC |
| | CCTCTATTTTCCATCCGTCAATCCATCCATCCATCCATCCATCCATCCATCCATCCAT |
| | CCATCAATCCATCCAAGGGAGTATCAGAGCTTTGTGAACATATATTGGTTGCTAATT |
| | ATGGACAAATGGATTTCCTGTATATGCTGATCTGGGTGGTATTTATATCAGTAATGT |
| | GAATGAAGACCTATGTGATTTTCATGTTGCATTTGTAATACATTGTTATGTAGTATTT |
| | TAAATGCCTAGTCTGTTAGAGCTACTGTAATAAAGTATCACAAACAGAGCAGCTTA |
| | AACAACAGAAATTTATTGTCCCTCAGTTCTGGAGGCCAGAAGTGTGAGATTAAGAT |
| | GCTAGCAGGCTGGGTCCTTCTGAGGGCTGTGGGGAATCTGTTCATGCTTCTCTTTGG |
| | CTTCTGGTGGAATGTCATCTTGCTGACACATTTTGCCATTCCTTGACCTGTAGATGCC |
| | TCTGCCACACCATTGCCTTTATCTCTACGTGGTGTCTTCCCTGTGTCTGTGTGTGTCTT |
| | TCTCCATGTCCCAAGTTTCCCTGTGAAAGAACACAAGTCATGTTAGATTTGTAACGA |
| | CCTGATCTTAACTAATTACATTTTCAGTAACCGTATCACCAAATAAGGTCACAATCT |
| | GAAGTACTGGGGCCTAGGACTTCCACATATAAAGTCTTAGAGGAAACAATTCAATC |
| | CATAACACCCTATCTATTGAAACTAGAACCATAATATAAATATTTTTCTGTGATATC |
| | CTTTTTTCATTTATCATGGATAAATTTCCATGCTGATTTGTGTAGATCTATCGATTGG |
| | GGCATAATATCCCATAGTGTGCTTTACCATAATTTATTTAATCAAATCATGCATTTTG |
| | GGATAACTGTTCTTTTCCACTATTACAAACAACTCTGCAAAAAGCACATTTGTATAT |
| | ATAGTTTTATGCATGTGTATTTGCTTTGAGATAGGTATCTATAAGTGGCATTAATCA |
| | GTCGAGACTTTTATGCATTTTATGTGTTAGGTACAGCCATACATGTGGCTGTGTGTTA |
| | GGTATAGCCTATGGGTTAGGCATAGCCTGTGTGTTAGGTATAGCCTATGGGTTAGGT |
| | ATAGCCTACGTGTTAGGTATAGCCTATGTGTTAGGTATAGCCTATGTGTTAGGTATA |
| | GCTACATTTTTTTCTCTCGAACTTTGGGTCCTCATCTCTGGCTGCATATTGTGTACTG |
| | CCTGTCTCAATACTGCTCAAGGGTCACACATGCTGAGCATTTGCAACATTGGTTTAA |
| | TTATCATACCCCACCTTCCACCCCCGTTCTCTGACCTGCTCCTGCCCTCTTCCTCCTG |
| | CCTCATTGAATAGTGGGGCTTTCCAGGAGGCTGCTGAGTGAGTAAACGGGGGACGG |
| | TCCTAAATGCTCTGCTTTCGGTTCCCTCTACCACTCATCACTACACCAGAGGGAACTT |
| | TTTAAGATGCAAATTTGAAAATATTGACCCTTTGTTCAAAAATCTTCAAAAGTTCTT |
| | CTCTGCAGTCAGAGTGACATCTTAGCTTGGCACACAAGGCTGTCCCTGATACTGCCC |
| | TCTCTCCTGCCATACCTTGCACTCCATTTTGAGTCATATTGGATGAGTTTTGTTTTGC |
| | TGTTTTAGAGATGGGGACTTGCTATGATGCTCAGGCTGAATTTGAATTCCTGGGTTC |
| | ATCTACAGCCATACCACCCAGAGTGCCTGATCTCATCTGCACTCCTGGGCTCAAGCA |
| | ATCCTCCCGTCTCAGCCGCCCAAGTAGCTGGGACTATAGGTGGACATCACTGTGCCC |
| | AGCTATGAATTAGTTTTGAAACATCTTGGTGGTTCTTTTAATCTTCCTTATTCTTGCT |
| | ACTCTTTTCCTTTTGCCTGGAAGGTTACCCCCACTTTCCCACTTTCTACTATCCATCTA |
| | AGAAGCTCCTACTTATTTTTTAATTACTGTGATCCTCATTTTGTCCATTTAAAAGCAG |
| | GGATAAAACTAACATTCCCATCTTGAGGTTGTGATCATTCAATGAGATAACCCTGTT |
| | TAAAGTCGTCATCCATGTCAAGCACACAATAAATACTCAAAAAATGTTTGATCTTCT |
| | TCTTCAGATTCCTTCCTTCCTTCCTTCCTTCTTCCTCCTCCTCCTTTCTTTCTCTCTTTT |
| | TCTTTTTCTCTTTCTTTCTTTCTTTCTCTCTCTCTCTCTTTCTTTCTTTCTCTTTCTCCCT |
| | TCCCTTCCCCTCCCCCCCCCCTTTCCCTTTCCCCTTTCCTTTCCTTATTTTCCTTTCTTT |
| | TCTTTTCTTTTTTTAAGACAGGGTTTTGCTCTGTGGCTCAGACTGGAGTGCAGTGGTA |
| | GGATCATAGCTCACTGTAGCCTTGAACTCTCCTGGGCTCAAGCAATCTTCCTGCCTC |
| | AGCCTTCTGACTAGCTAGGACTACAGGTGTGTACTACCATGCCTGGGTAATGTTTAA |
| | AATGTTTAAAATTTTTTATGTAGAGACAGGGTCTTGCTATGTTGCCCAGGCTGGTCT |
| | CAAACTCCTGGCCTCAAGTGGTCCTCCCACCTTGGCCTCCCAAAGCACTGGGATTCA |
| | GTTTTCATTATGAGACAGTTTTGTGACATAGGCAGGTAGTTAATAAATAAAAATCCC |
| | CAACCAATTAACTTAATACAGTACATGGCAAGATATCCGTGCTTGGTAAATGTAGA |
| | GCTCAGTTAGGTGGATTGGTTGAAATCGAGATGTCAATCACATTCCTCTAATCCAAG |
| | GATTGGTTGGTGGGACATGGTTGTTTGATTCTTTAAACATTTTATTATGTGTTCAGTG |
| | TAACCGTTGCTTAATAGTTACCAGCATTTTCACAATTTGGATATGACGTTTTCTTAAC |
| | AAAACTGCACCGTCTGTTGCAATTTCTACTACTACTGTGTAGTGCTCCCCAAACATC |
| | TGTTGAATAATCCATTCTACTCCTTTTTGGAAATAGTGGTAAAGCCTGCATAACTTTA |
| | ATTACATAATTTAAAAAATATTTTCTCTTTTCCTCTTTGAAGACCATCCCCATTCTAC |
| | TAGAATGTCCCTGGCCTTTGCCAGGAATGCTACTAATCCCTTCTGCATGTTAATTAA |
| | CAACAACCCTCTGATAGAATTCTTACCGGGGATGAGAGCTTAGTAGAAGCCACTCA |
| | AAACTTGCTTTGTTTTTGTCTGTCTCTCTTGGGCTTCATTTGCCTATTAGTCTTACTTA |
| | CTGTGTCTTTTCAGTTTGACTCTTCCTTAATTGGGAAGATAGAAACCAGTGGATGGA |
| | TAATCACTTTTTCCTGCTGGTCATCTGTTCATGCTTTACTGTCTGAATACACTTTTTCT |
| | GTATTTCTATTTTTCCTGCCTCGTAGACACCAAAGAGCTTAAAAAACATGCCATTGC |
| | CAAGGACTCTGGCAGGAAGGAGGCAATCACCTGCGCTATTCCTGGCTTCATGGAAT |
| | AGAAGCATCTGTGCTCCTAGGAATTACGCCACCAGTGATTTTAGAGCTCTTATGCTC |
| | TTCCTGGGTGATAGAATGGGCTTAATTTGACTCAAGAATAGTACATCAAAACTCTTA |
| | ATTCCATCGAGATTGATGCATTTTTGGGGTTTTATGCTCTAAAAGGCCTTTTGCTGTG |
| | CAGAAGGAAGAGAGAGGTGGACAGAAAGGGCAGTGGGGAGAAGATGAGACTAAA |
| | AGGTGGAAAGTCCAGGTCTGGATCCCTCCCCACCACACACCTGCTCTGTGGCCTTGA |
| | GAGAAACATGTAATTTTTATAAGCCTTAGATTTCTAATATGTAAAATGTATGTAATA |
| | GTTTTAGCTGCACTCCTTCCTTGCCAGGTTGATATAATAATCAAATAAGACTGGTCT |
| | CTGGGTCAGGCACAGTGGCTCATGCCTGTAATCCCAGCACTTTGGGAGGCCAAGGT |
| | GGGTGGATTGTCTGAGCCCAGGAGTTTAAGACTGCAGTGAGCTGTGCACTTGGCCTG |
| | GGTGACTGAGCAAGAGCCTGGCTTTTTCTATTTATTTATTTATTTTTGAGACAGAGTC |
| | TCACTTTGTTGCCCAGGTTGAAGTGCAGTGGCAATCTCGGCTCACTGCAACCTCTGC |
| | CTCCCAGGTTCAAGCAATTCTCCTGCCTCAGCCTCCCAAGTAGCTGGGATTACAGGT |
| | GCCCACCACCACCCTAACTAATTTTTATATTTTTAGTAGAGATGGGCTTTCACCATGT |
| | TGGCCAGGCTGGTTTTGAACTCCTGACCTCGTGATCCACCCTCCCAAAGTGCTGGAC |
| | CTCCCAAAGTGCTGGGATTACAGGCATAAGCCACTGCACCTGGCCTTATTTATTTAT |
| | TTATTTAGGCAGGGTCTCACTCTGTCACCCAGGCTAGAGTGTGGCAGCGCAATCATG |
| | GCTCACTGCAGCCTCGACTTCCTGGACTCAAGCGATCCTCCCACCTCTCCAACGTGG |
| | TTGAGGGCTGCAGCGAGAAAGATGTGAAAGGGGCATTTGGGGGCGCAGGAAGGCG |
| | GGAGGTGGCAATTGCTAAGGGCCCCGCCGTGTTCCTGTCACAGCCCTAGCTACAGCT |
| | TAGAAGGTCACAATGGACGTTCACCTGTTGTCTTTGATGTTTAAATCAGCACCGACT |
| | TCCCATGAGGAGGATTTTTTTTTCCTGTCTCTCTTAAAAAAAAAAAAAAAGGCCCCT |
| | GCAGTGTGGGTGTCTGCAGTGTTACGGGGATTATTAGTTGAGGATAACCACTGGGAT |
| | GTGTTTCTGGAAGAAGTGGTGCTGAGGAGCACTCCTGGCCGGAGGAAAGCTGTGTG |
| | AGGAGGAGGAGGAGTATGGGGTAACTGGCTCAGTTTGCCTTGGCTCTTTCTGTCGGA |
| | CTCATACAGCAAAGCATCTGCTGAGACATCATTTGTGTCTCCTGTGACAGCCAGAGA |
| | CAGCAGGTCTTGCTCTCCCCAGATCATATCTGAGATGCTGGCTCCTGCTCTCTGAGC |
| | TGAAGGCTGCAGCTGCTGAGAGAGGACGAGGCACTGAGTCAAAGCAGCTGCACAAT |
| | GTTGGGGAACAGCTGGGTACTGAGGACTCTTCTCCCTGTCCTTCTACAGGTTGGTGA |
| | ATTCTGGCCTTACGTCAGTCTGTTGTTCAGCTTTGTCCTCGGTACTCAGCACTAATCA |
| | GAATCTCACGCACCTTTACCTGCGAGGCAACACTCTCGGAGACAAGGGGATCAAAC |
| | TACTCTGTGAGGGACTCTTGCACCCCGACTGCAAGCTTCAGGTGTTGGAGTAAGTCC |
| | TTTGGTTTATTACAGCAATGAGAACACATGGTGGCCAAGGGTGGAGGGACGTTTAG |
| | GCAGAGTGGCCACAAGATAATCTGTATCTTAGAAGTGAGGTGTCTTCTTAGTGTTTG |
| | CCTTGTTACAGGATCATGGGACTGGGAGGAGTCCTTCCAGATGATATAGATGTAGG |
| | AACTTGAGTCCTTACCATCACAGTTTTCTGTACCACAAAACAAGGAGGGCAGTATGA |
| | CACTTACATTTTATGGAAATGTAGGGAAAGGCACAGAATTTGGTAAATCAATGAAC |
| | AAACCACTGTTACTAATTTGATCTGTGCAATGCAGTGGATTTGAAAAAGAGAGAGA |
| | AGAATAATTACCAGCATACAAGGCTGCTTGAAGCTAGTTAGTCCTGTGCTCCTGTGC |
| | TTTTTTTTTTTCCACCTGAAACAAGACAGGCTAAGATGCTAAAATCTTGGGGAGCTA |
| | GGGGGATGGTTAAGGGGACATTTTCTTTAAATCACCCCCTTTTTGCAGATTAGACAA |
| | CTGCAACCTCACGTCACACTGCTGCTGGGATCTTTCCACACTTCTGACCTCCAGCCA |
| | GAGCCTGCGAAAGCTGAGCCTGGGCAACAATGACCTGGGCGACCTGGGGGTCATGA |
| | TGTTCTGTGAAGTGCTGAAACAGCAGAGCTGCCTCCTGCAGAACCTGGGGTGAGTG |
| | TGCTCTGCAGAGATGCCCGTGGTGGGACTCTGAGTTCTCAGGAAACGTTGACTGTTA |
| | TCAAAATCCAGGATGGCTCTCGCTTCATTTGCAGCCACTCAAGATTAGGTTGGGCCT |
| | GGGTCAGTTGTGGTGGATATTTTAAAATAGAGAATATGGGGTTAAACTACACATTCC |
| | ACTTCACTGAGATGGTTAGAATCACCAAGGCTAAGCTGGATTTCACCCACATTCCCC |
| | AAAGAAACACAAGCCCTCATGCGATCATGGCCAAGGAGGATTCTGTTCTACACAAG |
| | GAACCGAACTTCGTAGAACTCGTCTATGGGTCATCCAAATTGGAAGGGTCTTTACAT |
| | AGTGGGGATCTTGTTGTTTTGTTTCTGAGGATTTATTTTTTTTTTCCTTGAGACAGAG |
| | TCTTGCTCTGTCGCCCAGGCTGGAGTGCAGTGGCATGATCTCGGCTCACTGCAAGCT |
| | CCGCCTCCCGGGTTCACGCCATTCTCCTGCCTCGGCCTCCCAAGTAGCTGGGACCAC |
| | AGGCGCCTGCCACCACACCCGGCTAATTTTTTGTATTTTTAGTAGAGTTGGGGTTTC |
| | ACTGTGTTAGCCAGGATGGTCTCCATCTCCTGACCTCGTGATCTGCCTGCCTCGGCCT |
| | CCCCAAGTGCTGGGATTACAGGTGTGAGCCTCCGTGCCTGGCGGTTTCTGAGGATTT |
| | CTAAATGGGAAAAGAGTGCTCCAGGAATAGCAAGATGGGAAATTACACGAAATTCG |
| | ACAGTGTGTCAGGATATTGCCAGCCCACCTCTGTGATACTACTGAGGTAGTCGTCTC |
| | CTCTCTAGCAAGAGCTTCCTGGCGAGTCCCACGGTTCTGTCAGCCGTGATGATTGCA |
| | CTTTTCCCTGAAGACGTTTTCACTGCTTGGCTTTAAGGCCAACATGCTCTCCTGGGTT |
| | TTCTCCTATTTCATCAGATCCTTCTACTCAGTCTCCTTTCTGGCCTCTTCTTCATCATC |
| | CTGACCCTTATGTGTTGAATCCTCAGAGCTTAGTCGCTGGGCTAATTTTTCTTCCCTA |
| | CACTTTCTCCCTCATTTAAATTATCTGGTCTTCTGGCTTCATGATATGTGTGCTGTGG |
| | CCTCCAGATTATATGTCCATAAATTCTGTGTCTCCGTGCTTGGCTGATCTTTCTTCTT |
| | GAAGGTTTAATAAACCTCTCAAACGTCACATCTAGATCCACACCCTTGCTACTCTCT |
| | TCTACCCGAAACCTGATTCTTCCCCATTTCAGTCGATGGTCACTTCCTCCAAGTTGCT |
| | CGGGCCAAAACATTTCTGGTTCTGTCGTCACTGTCTCTTTTTCCTTATAATCATGTCC |
| | AATGTGTCAGCACATCCTGTGAGCTCTGCCTTCCCAGCTTGGCCACTCCCTGACCAC |
| | TTCAGGCCAGCTTCACGGCTTCTTCCTTGGCTCAAGCCACCATCACCTGGTGTCTGG |
| | ATTGTCATGGTGGCATTCTGACAGGTCACCCTGCATCTGTTATTCTGATTTTAGCAGA |
| | CAAGGGATTCAGCTAAAACGTAACTCGCATCATATCACTCTTGTCAACATCGTCTTC |
| | TTATCACTTTCATAATCAGAGCAACAAACTCATGGTGGCTTCCATGCCCCACATAAT |
| | CCAGCCTCTCCTGCTTCCGAAGATTCCGCTTCTCCCAACCCTTGCTTATTCTGCCCCA |
| | GTACAGTGATCTCCTTGCTGTTTTGCAAGAACACTGTCATCTCTGGATTTTTGCACTT |
| | GCTTTTCTTCTGGCCTAGAACTCTCTCCCCTTCAGTATCCACCTGCATTATTCCCTGT |
| | ACCTTCCTAAATTCTCTGCTTAAAGGTCTTCTCTGCGAGGCTGTCTTTGACTATTCTA |
| | CTGAAAATAGAAGCACTTTTTCTTCTCTTCTCATTCTCCTCTGCAGTGTAGCACATCT |
| | TATGTGCTATGTCATTTTCTTGTTTATTTATTATCTATCTCTGCTCCCAAAATATAAAT |
| | TCCATTAGTCCAGGGACTTCTGTCCATATTAATGATCTCAAGGCTGAGAACTTGTTG |
| | GAATGCAGGTTTGAATAAATACAAGTTTGAATCAGCCTTGCATCCTTGTGCCAACAT |
| | AATGGAGCACATTTGCTGCAGGACAAGCCAATATTCCCAGAGCTAAGAGAGATGGG |
| | GGAACAAACAAAGGATTGGTCAGGTCAAAGCTGGGGACACTGCTACATGTAGCATC |
| | ACTGACCTAGCCCTGAGGGACATTCTGGACCCAGGCTGAGGGACAGGCTCAAAATA |
| | GATTGAAGAATCCATGAATGCAATCAACCCGTTTTCTCAGGCTGCGATAACAAGCTG |
| | CTTTAGACTGTGTGACTCAAACACAGATTTATTATCTCACAATACTGGAGGCTAGAA |
| | AGTCCAAAATCAAGTCTCTGATGAATTTGGTTGCTGGCGAGAGTCCTCTTCCTGATT |
| | TGCAGATGGCTACCTTCTTGTTATGTGATTGCATGGAGGGAGGGGAGGCAAGGGAG |
| | AGAAGACAGGGGAGAAAGAACAAACTCTCTGATATCTCTCCTGTAAGGGCACTCAC |
| | CCCATACTGAGGGCCCCACCCTCACAACTTCATCTAACTCTAACTACTTCCCAAGGG |
| | CCTCACCTTTAAGTACCGTACATTAGGGGTTCGGGCTTCATCATGGGAATTGTTAAG |
| | GAAGACAAACATTCAGTATATAACACCACCAAATTGAGATGGTTATAAATGGTGCA |
| | GGACAAATGGGTGGATTTATTAGTTAAGTTGCAAGGGAAACTTTATTAAAGAGGAG |
| | GAAGAACTTACATCGAAGATGTGAGAGGCATCCTGGGCAAATGCAATGTGTAGAAT |
| | TGATCAGGTTAGTGACTAAATGAACATAAGGTAAAAAAATTTACAAGACAGTTGAG |
| | GAAGCGTAAATGCTGACTGGACATACAGTGATACTAAGAAATTATTGTTAAACGAT |
| | TTTGGTGTGATAGTGACATTGTAATTATATTTTAGTGTCACAAAGTGAATTATTGAT |
| | AGATGCATTAAATGCAGTATTTAAAATAATCCATGGGTGGGAGGAGTGGGTGGGCA |
| | ATAGAAGAAACAAGTTGACATGACCTGTTTAATTGTCGAACTTGGGTAATGGACAC |
| | AGGGGGTTCATTATACTATTTTCTATACTTTTATGTATGCTTAACATTTTCCCAACTA |
| | GATAGTTAAAAATATAACAGGAACCACATTGGGCACTTAGAGCTTTTTTTTTTTTTTC |
| | CCCACATTTTTATTGGGAGCCGTGGGAGGGGCCTCCTCTGTCATTGGAGGTGCTCAC |
| | AGTTTCTTCAGCCACTCCAGGCTGGGGTCCTGGGGGTCTGAGGCTCCCAGTGACGAA |
| | GGACACGACGAGCACCGGCTCCTTGGCCCAGGCATTCTTGAAGAAGGTGCGGAGTC |
| | TCGCGGCCATCTTGGTCTCGGCGGCGGCGACGGCGGCGAGGACGCGGAGCACTCTG |
| | GGAGTTGTGGTCCCTCTTTTTTTTTTTTTTTTTTTTTTACATTTTAGAAGTGTGTGGAG |
| | TTTAGGGAAATGAAGAGATGAGACTTTTAAGGTTAACGACTCTGACAGAGTTATCT |
| | GAAGAGTGCAACCCAGGCTTTCTATTTGCTTTTACAGGTTGTCTGAAATGTATTTCA |
| | ATTATGAGACAAAAAGTGCGTTAGAAACACTTCAAGA |
| 54 | GTTCCTGAGGCTGGCATCTGGATGAGGAAACTGAAGTTGAGGAATAGTGAAGAGTT |
| | TGTCCAATGTCATAGCCCCGTAATCAACGGGACAAAAATTTTCTTGCTGATGGGTCA |
| | AGATGGCATCGTGAAGTGGTTGTTCACCGTAAACTGTAATACAATCCTGTTTATGGA |
| | TTTGTTTGCATATTTTTCCCTCCATAGGGAAACCTTTCTTCCATGGCTCAGGACACAC |
| | TCCTGGATCGAGCCAACAGGAGAACTTTCTGGTAAGCATTTGGCTAACTTTTTTTTTT |
| | TTGAGATGGAGTCTTGCTGTGTCGCCTAGGCTGGAGTGCAGTGGCGTGATCTTGGCT |
| | CACTGCAGCCTCCACTTCCCGGGTTCAATCAATTCTCCTACCTCAACTTCCTGAGTAG |
| | CTGGGATTACAGGCGCCCGCCACCACACCCGGCTCATTTTTGTACTTTTAGTAGAGA |
| | CACAGTTTTGCCATGTTGGCCAGGCTGGTCTTGAATTCCTCAGCTCAGGTGATCTGC |
| | CTGCCTTGGCCTCTCAAAGTGCTGGGATTACAGGCGTGAGCCACTGTGCCCGGCCTT |
| | GGCTAACTTTTCAAAATTAAAGATTTTGACTTGTTACAGTCATGTGACATTTTTTTCT |
| | TTCTGTTTGCTGAGTTTTTGATAATTTATATCTCTCAAAGTGGAGACTTTAAAAAAGA |
| | CTCATCCGTGTGCCGTGTTCACTGCCTGGTATCTTAGTGTGGACCGAAGCCTAAGGA |
| | CCCTGAAAACAGCTGCAGATGAAGATGGCAAGCACCCGCTGCAAGCTGGCCAGGTA |
| | CCTGGAGGACCTGGAGGATGTGGACTTGAAGAAATTTAAGATGCACTTAGAGGACT |
| | ATCCTCCCCAGAAGGGCTGCATCCCCCTCCCGAGGGGTCAGACAGAGAAGGCAGAC |
| | CATGTGGATCTAGCCACGCTAATGATCGACTTCAATGGGGAGGAGAAGGCGTGGGC |
| | CATGGCCGTGTGGATCTTCGCTGCGATCAACAGGAGAGACCTTTATGAGAAAGCAA |
| | AAAGAGATGAGCCGAAGTGGGGTTCAGATAATGCACGTGTTTCGAATCCCACTGTG |
| | ATATGCCAGGAAGACAGCATTGAAGAGGAGTGGATGGGTTTACTGGAGTACCTTTC |
| | GAGAATCTCTATTTGTAAAATGAAGAAAGATTACCGTAAGAAGTACAGAAAGTACG |
| | TGAGAAGCAGATTCCAGTGCATTGAAGACAGGAATGCCCGTCTGGGTGAGAGTGTG |
| | AGCCTCAACAAACGCTACACACGACTGCGTCTCATCAAGGAGCACCGGAGCCAGCA |
| | GGAGAGGGAGCAGGAGCTTCTGGCCATCGGCAAGACCAAGACGTGTGAGAGCCCC |
| | GTGAGTCCCATTAAGATGGAGTTGCTGTTTGACCCCGATGATGAGCATTCTGAGCCT |
| | GTGCACACCGTGGTGTTCCAGGGGGCGGCAGGGATTGGGAAAACAATCCTGGCCAG |
| | GAAGATGATGTTGGACTGGGCGTCGGGGACACTCTACCAAGACAGGTTTGACTATC |
| | TGTTCTATATCCACTGTCGAGAGGTGAGCCTTGTGACACAGAGGAGCCTGGGGGAC |
| | CTGATCATGAGCTGCTGCCCCGACCCAAACCCACCCATCCACAAGATCGTGAGAAA |
| | ACCCTCCAGAATCCTCTTCCTCATGGACGGCTTCGATGAGCTGCAAGGTGCCTTTGA |
| | CGAGCACATAGGACCGCTCTGCACTGACTGGCAGAAGGCCGAGCGGGGAGACATTC |
| | TCCTGAGCAGCCTCATCAGAAAGAAGCTGCTTCCCGAGGCCTCTCTGCTCATCACCA |
| | CGAGACCTGTGGCCCTGGAGAAACTGCAGCACTTGCTGGACCATCCTCGGCATGTG |
| | GAGATCCTGGGTTTCTCCGAGGCCAAAAGGAAAGAGTACTTCTTCAAGTACTTCTCT |
| | GATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCA |
| | GATGGAGAGTGGCAAGAGCCTTGCCCAGACATCCAAGACCACCACCGCGGTGTACG |
| | TCTTCTTCCTTTCCAGTTTGCTGCAGCCCCGGGGAGGGAGCCAGGAGCACGGCCTCT |
| | GCGCCCACCTCTGGGGGCTCTGCTCTTTGGCTGCAGATGGAATCTGGAACCAGAAA |
| | ATCCTGTTTGAGGAGTCCGACCTCAGGAATCATGGACTGCAGAAGGCGGATGTGTC |
| | TGCTTTCCTGAGGATGAACCTGTTCCAAAAGGAAGTGGACTGCGAGAAGTTCTACA |
| | GCTTCATCCACATGACTTTCCAGGAGTTCTTTGCCGCCATGTACTACCTGCTGGAAG |
| | AGGAAAAGGAAGGAAGGACGAACGTTCCAGGGAGTCGTTTGAAGCTTCCCAGCCG |
| | AGACGTGACAGTCCTTCTGGAAAACTATGGCAAATTCGAAAAGGGGTATTTGATTTT |
| | TGTTGTACGTTTCCTCTTTGGCCTGGTAAACCAGGAGAGGACCTCCTACTTGGAGAA |
| | GAAATTAAGTTGCAAGATCTCTCAGCAAATCAGGCTGGAGCTGCTGAAATGGATTG |
| | AAGTGAAAGCCAAAGCTAAAAAGCTGCAGATCCAGCCCAGCCAGCTGGAATTGTTC |
| | TACTGTTTGTACGAGATGCAGGAGGAGGACTTCGTGCAAAGGGCCATGGACTATTT |
| | CCCCAAGATTGAGATCAATCTCTCCACCAGAATGGACCACATGGTTTCTTCCTTTTG |
| | CATTGAGAACTGTCATCGGGTGGAGTCACTGTCCCTGGGGTTTCTCCATAACATGCC |
| | CAAGGAGGAAGAGGAGGAGGAAAAGGAAGGCCGACACCTTGATATGGTGCAGTGT |
| | GTCCTCCCAAGCTCCTCTCATGCTGCCTGTTCTCATGGATTGGTGAACAGCCACCTC |
| | ACTTCCAGTTTTTGCCGGGGCCTCTTTTCAGTTCTGAGCACCAGCCAGAGTCTAACT |
| | GAATTGGACCTCAGTGACAATTCTCTGGGGGACCCAGGGATGAGAGTGTTGTGTGA |
| | AACGCTCCAGCATCCTGGCTGTAACATTCGGAGATTGTGGTTGGGGCGCTGTGGCCT |
| | CTCGCATGAGTGCTGCTTCGACATCTCCTTGGTCCTCAGCAGCAACCAGAAGCTGGT |
| | GGAGCTGGACCTGAGTGACAACGCCCTCGGTGACTTCGGAATCAGACTTCTGTGTGT |
| | GGGACTGAAGCACCTGTTGTGCAATCTGAAGAAGCTCTGGTTGGTCAGCTGCTGCCT |
| | CACATCAGCATGTTGTCAGGATCTTGCATCAGTATTGAGCACCAGCCATTCCCTGAC |
| | CAGACTCTATGTGGGGGAGAATGCCTTGGGAGACTCAGGAGTCGCAATTTTATGTG |
| | AAAAAGCCAAGAATCCACAGTGTAACCTGCAGAAACTGGGGTTGGTGAATTCTGGC |
| | CTTACGTCAGTCTGTTGTTCAGCTTTGTCCTCGGTACTCAGCACTAATCAGAATCTCA |
| | CGCACCTTTACCTGCGAGGCAACACTCTCGGAGACAAGGGGATCAAACTACTCTGT |
| | GAGGGACTCTTGCACCCCGACTGCAAGCTTCAGGTGTTGGAATTAGACAACTGCAA |
| | CCTCACGTCACACTGCTGCTGGGATCTTTCCACACTTCTGACCTCCAGCCAGAGCCT |
| | GCGAAAGCTGAGCCTGGGCAACAATGACCTGGGCGACCTGGGGGTCATGATGTTCT |
| | GTGAAGTGCTGAAACAGCAGAGCTGCCTCCTGCAGAACCTGGGGTTGTCTGAAATG |
| | TATTTCAATTATGAGACAAAAAGTGCGTTAGAAACACTTCAAGAAGAAAAGCCTGA |
| | GCTGACCGTCGTCTTTGAGCCTTCTTGGTAGGAGTGGAAACGGGGCTGCCAGACGCC |
| | AGTGTTCTCCGGTCCCTCCAGCTGGGGGCCCTCAGGTGGAGAGAGCTGCGATCCATC |
| | CAGGCCAAGACCACAGCTCTGTGATCCTTCCGGTGGAGTGTCGGAGAAGAGAGCTT |
| | GCCGACGATGCCTTCCTGTGCAGAGCTTGGGCATCTCCTTTACGCCAGGGTGAGGAA |
| | GACACCAGGACAATGACAGCATCGGGTGTTGTTGTCATCACAGCGCCTCAGTTAGA |
| | GGATGTTCCTCTTGGTGACCTCATGTAATTAGCTCATTCAATAAAGCACTTTCTTTAT |
| | TTT |
| 55 | GTTCCTGAGGCTGGCATCTGGATGAGGAAACTGAAGTTGAGGAATAGTGAAGAGTT |
| | TGTCCAATGTCATAGCCCCGTAATCAACGGGACAAAAATTTTCTTGCTGATGGGTCA |
| | AGATGGCATCGTGAAGTGGTTGTTCACCGTAAACTGTAATACAATCCTGTTTATGGA |
| | TTTGTTTGCATATTTTTCCCTCCATAGGGAAACCTTTCTTCCATGGCTCAGGACACAC |
| | TCCTGGATCGAGCCAACAGGAGAACTTTCTGGTAAGCATTTGGCTAACTTTTTTTTTT |
| | TTGAGATGGAGTCTTGCTGTGTCGCCTAGGCTGGAGTGCAGTGGCGTGATCTTGGCT |
| | CACTGCAGCCTCCACTTCCCGGGTTCAATCAATTCTCCTACCTCAACTTCCTGAGTAG |
| | CTGGGATTACAGGCGCCCGCCACCACACCCGGCTCATTTTTGTACTTTTAGTAGAGA |
| | CACAGTTTTGCCATGTTGGCCAGGCTGGTCTTGAATTCCTCAGCTCAGGTGATCTGC |
| | CTGCCTTGGCCTCTCAAAGTGCTGGGATTACAGGCGTGAGCCACTGTGCCCGGCCTT |
| | GGCTAACTTTTCAAAATTAAAGATTTTGACTTGTTACAGTCATGTGACATTTTTTTCT |
| | TTCTGTTTGCTGAGTTTTTGATAATTTATATCTCTCAAAGTGGAGACTTTAAAAAAGA |
| | CTCATCCGTGTGCCGTGTTCACTGCCTGGTATCTTAGTGTGGACCGAAGCCTAAGGA |
| | CCCTGAAAACAGCTGCAGATGAAGATGGCAAGCACCCGCTGCAAGCTGGCCAGGTA |
| | CCTGGAGGACCTGGAGGATGTGGACTTGAAGAAATTTAAGATGCACTTAGAGGACT |
| | ATCCTCCCCAGAAGGGCTGCATCCCCCTCCCGAGGGGTCAGACAGAGAAGGCAGAC |
| | CATGTGGATCTAGCCACGCTAATGATCGACTTCAATGGGGAGGAGAAGGCGTGGGC |
| | CATGGCCGTGTGGATCTTCGCTGCGATCAACAGGAGAGACCTTTATGAGAAAGCAA |
| | AAAGAGATGAGCCGAAGTGGGGTTCAGATAATGCACGTGTTTCGAATCCCACTGTG |
| | ATATGCCAGGAAGACAGCATTGAAGAGGAGTGGATGGGTTTACTGGAGTACCTTTC |
| | GAGAATCTCTATTTGTAAAATGAAGAAAGATTACCGTAAGAAGTACAGAAAGTACG |
| | TGAGAAGCAGATTCCAGTGCATTGAAGACAGGAATGCCCGTCTGGGTGAGAGTGTG |
| | AGCCTCAACAAACGCTACACACGACTGCGTCTCATCAAGGAGCACCGGAGCCAGCA |
| | GGAGAGGGAGCAGGAGCTTCTGGCCATCGGCAAGACCAAGACGTGTGAGAGCCCC |
| | GTGAGTCCCATTAAGATGGAGTTGCTGTTTGACCCCGATGATGAGCATTCTGAGCCT |
| | GTGCACACCGTGGTGTTCCAGGGGGCGGCAGGGATTGGGAAAACAATCCTGGCCAG |
| | GAAGATGATGTTGGACTGGGCGTCGGGGACACTCTACCAAGACAGGTTTGACTATC |
| | TGTTCTATATCCACTGTCGAGAGGTGAGCCTTGTGACACAGAGGAGCCTGGGGGAC |
| | CTGATCATGAGCTGCTGCCCCGACCCAAACCCACCCATCCACAAGATCGTGAGAAA |
| | ACCCTCCAGAATCCTCTTCCTCATGGACGGCTTCGATGAGCTGCAAGGTGCCTTTGA |
| | CGAGCACATAGGACCGCTCTGCACTGACTGGCAGAAGGCCGAGCGGGGAGACATTC |
| | TCCTGAGCAGCCTCATCAGAAAGAAGCTGCTTCCCGAGGCCTCTCTGCTCATCACCA |
| | CGAGACCTGTGGCCCTGGAGAAACTGCAGCACTTGCTGGACCATCCTCGGCATGTG |
| | GAGATCCTGGGTTTCTCCGAGGCCAAAAGGAAAGAGTACTTCTTCAAGTACTTCTCT |
| | GATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCA |
| | GATGGAGAGTGGCAAGAGCCTTGCCCAGACATCCAAGACCACCACCGCGGTGTACG |
| | TCTTCTTCCTTTCCAGTTTGCTGCAGCCCCGGGGAGGGAGCCAGGAGCACGGCCTCT |
| | GCGCCCACCTCTGGGGGCTCTGCTCTTTGGCTGCAGATGGAATCTGGAACCAGAAA |
| | ATCCTGTTTGAGGAGTCCGACCTCAGGAATCATGGACTGCAGAAGGCGGATGTGTC |
| | TGCTTTCCTGAGGATGAACCTGTTCCAAAAGGAAGTGGACTGCGAGAAGTTCTACA |
| | GCTTCATCCACATGACTTTCCAGGAGTTCTTTGCCGCCATGTACTACCTGCTGGAAG |
| | AGGAAAAGGAAGGAAGGACGAACGTTCCAGGGAGTCGTTTGAAGCTTCCCAGCCG |
| | AGACGTGACAGTCCTTCTGGAAAACTATGGCAAATTCGAAAAGGGGTATTTGATTTT |
| | TGTTGTACGTTTCCTCTTTGGCCTGGTAAACCAGGAGAGGACCTCCTACTTGGAGAA |
| | GAAATTAAGTTGCAAGATCTCTCAGCAAATCAGGCTGGAGCTGCTGAAATGGATTG |
| | AAGTGAAAGCCAAAGCTAAAAAGCTGCAGATCCAGCCCAGCCAGCTGGAATTGTTC |
| | TACTGTTTGTACGAGATGCAGGAGGAGGACTTCGTGCAAAGGGCCATGGACTATTT |
| | CCCCAAGATTGAGATCAATCTCTCCACCAGAATGGACCACATGGTTTCTTCCTTTTG |
| | CATTGAGAACTGTCATCGGGTGGAGTCACTGTCCCTGGGGTTTCTCCATAACATGCC |
| | CAAGGAGGAAGAGGAGGAGGAAAAGGAAGGCCGACACCTTGATATGGTGCAGTGT |
| | GTCCTCCCAAGCTCCTCTCATGCTGCCTGTTCTCATGGGTTGGGGCGCTGTGGCCTCT |
| | CGCATGAGTGCTGCTTCGACATCTCCTTGGTCCTCAGCAGCAACCAGAAGCTGGTGG |
| | AGCTGGACCTGAGTGACAACGCCCTCGGTGACTTCGGAATCAGACTTCTGTGTGTGG |
| | GACTGAAGCACCTGTTGTGCAATCTGAAGAAGCTCTGGTTGGTGAATTCTGGCCTTA |
| | CGTCAGTCTGTTGTTCAGCTTTGTCCTCGGTACTCAGCACTAATCAGAATCTCACGC |
| | ACCTTTACCTGCGAGGCAACACTCTCGGAGACAAGGGGATCAAACTACTCTGTGAG |
| | GGACTCTTGCACCCCGACTGCAAGCTTCAGGTGTTGGAATTAGACAACTGCAACCTC |
| | ACGTCACACTGCTGCTGGGATCTTTCCACACTTCTGACCTCCAGCCAGAGCCTGCGA |
| | AAGCTGAGCCTGGGCAACAATGACCTGGGCGACCTGGGGGTCATGATGTTCTGTGA |
| | AGTGCTGAAACAGCAGAGCTGCCTCCTGCAGAACCTGGGGTTGTCTGAAATGTATTT |
| | CAATTATGAGACAAAAAGTGCGTTAGAAACACTTCAAGAAGAAAAGCCTGAGCTGA |
| | CCGTCGTCTTTGAGCCTTCTTGGTAGGAGTGGAAACGGGGCTGCCAGACGCCAGTGT |
| | TCTCCGGTCCCTCCAGCTGGGGGCCCTCAGGTGGAGAGAGCTGCGATCCATCCAGG |
| | CCAAGACCACAGCTCTGTGATCCTTCCGGTGGAGTGTCGGAGAAGAGAGCTTGCCG |
| | ACGATGCCTTCCTGTGCAGAGCTTGGGCATCTCCTTTACGCCAGGGTGAGGAAGACA |
| | CCAGGACAATGACAGCATCGGGTGTTGTTGTCATCACAGCGCCTCAGTTAGAGGAT |
| | GTTCCTCTTGGTGACCTCATGTAATTAGCTCATTCAATAAAGCACTTTCTTTATTTT |
| 56 | GTTCCTGAGGCTGGCATCTGGGGAAACCTTTCTTCCATGGCTCAGGACACACTCCTG |
| | GATCGAGCCAACAGGAGAACTTTCTGTGTGGACCGAAGCCTAAGGACCCTGAAAAC |
| | AGCTGCAGATGAAGATGGCAAGCACCCGCTGCAAGCTGGCCAGGTACCTGGAGGAC |
| | CTGGAGGATGTGGACTTGAAGAAATTTAAGATGCACTTAGAGGACTATCCTCCCCA |
| | GAAGGGCTGCATCCCCCTCCCGAGGGGTCAGACAGAGAAGGCAGACCATGTGGATC |
| | TAGCCACGCTAATGATCGACTTCAATGGGGAGGAGAAGGCGTGGGCCATGGCCGTG |
| | TGGATCTTCGCTGCGATCAACAGGAGAGACCTTTATGAGAAAGCAAAAAGAGATGA |
| | GCCGAAGTGGGGTTCAGATAATGCACGTGTTTCGAATCCCACTGTGATATGCCAGG |
| | AAGACAGCATTGAAGAGGAGTGGATGGGTTTACTGGAGTACCTTTCGAGAATCTCT |
| | ATTTGTAAAATGAAGAAAGATTACCGTAAGAAGTACAGAAAGTACGTGAGAAGCA |
| | GATTCCAGTGCATTGAAGACAGGAATGCCCGTCTGGGTGAGAGTGTGAGCCTCAAC |
| | AAACGCTACACACGACTGCGTCTCATCAAGGAGCACCGGAGCCAGCAGGAGAGGG |
| | AGCAGGAGCTTCTGGCCATCGGCAAGACCAAGACGTGTGAGAGCCCCGTGAGTCCC |
| | ATTAAGATGGAGTTGCTGTTTGACCCCGATGATGAGCATTCTGAGCCTGTGCACACC |
| | GTGGTGTTCCAGGGGGCGGCAGGGATTGGGAAAACAATCCTGGCCAGGAAGATGAT |
| | GTTGGACTGGGCGTCGGGGACACTCTACCAAGACAGGTTTGACTATCTGTTCTATAT |
| | CCACTGTCGAGAGGTGAGCCTTGTGACACAGAGGAGCCTGGGGGACCTGATCATGA |
| | GCTGCTGCCCCGACCCAAACCCACCCATCCACAAGATCGTGAGAAAACCCTCCAGA |
| | ATCCTCTTCCTCATGGACGGCTTCGATGAGCTGCAAGGTGCCTTTGACGAGCACATA |
| | GGACCGCTCTGCACTGACTGGCAGAAGGCCGAGCGGGGAGACATTCTCCTGAGCAG |
| | CCTCATCAGAAAGAAGCTGCTTCCCGAGGCCTCTCTGCTCATCACCACGAGACCTGT |
| | GGCCCTGGAGAAACTGCAGCACTTGCTGGACCATCCTCGGCATGTGGAGATCCTGG |
| | GTTTCTCCGAGGCCAAAAGGAAAGAGTACTTCTTCAAGTACTTCTCTGATGAGGCCC |
| | AAGCCAGGGCAGCCTTCAGTCTGATTCAGGAGAACGAGGTCCTCTTCACCATGTGCT |
| | TCATCCCCCTGGTCTGCTGGATCGTGTGCACTGGACTGAAACAGCAGATGGAGAGT |
| | GGCAAGAGCCTTGCCCAGACATCCAAGACCACCACCGCGGTGTACGTCTTCTTCCTT |
| | TCCAGTTTGCTGCAGCCCCGGGGAGGGAGCCAGGAGCACGGCCTCTGCGCCCACCT |
| | CTGGGGGCTCTGCTCTTTGGCTGCAGATGGAATCTGGAACCAGAAAATCCTGTTTGA |
| | GGAGTCCGACCTCAGGAATCATGGACTGCAGAAGGCGGATGTGTCTGCTTTCCTGA |
| | GGATGAACCTGTTCCAAAAGGAAGTGGACTGCGAGAAGTTCTACAGCTTCATCCAC |
| | ATGACTTTCCAGGAGTTCTTTGCCGCCATGTACTACCTGCTGGAAGAGGAAAAGGA |
| | AGGAAGGACGAACGTTCCAGGGAGTCGTTTGAAGCTTCCCAGCCGAGACGTGACAG |
| | TCCTTCTGGAAAACTATGGCAAATTCGAAAAGGGGTATTTGATTTTTGTTGTACGTT |
| | TCCTCTTTGGCCTGGTAAACCAGGAGAGGACCTCCTACTTGGAGAAGAAATTAAGTT |
| | GCAAGATCTCTCAGCAAATCAGGCTGGAGCTGCTGAAATGGATTGAAGTGAAAGCC |
| | AAAGCTAAAAAGCTGCAGATCCAGCCCAGCCAGCTGGAATTGTTCTACTGTTTGTAC |
| | GAGATGCAGGAGGAGGACTTCGTGCAAAGGGCCATGGACTATTTCCCCAAGATTGA |
| | GATCAATCTCTCCACCAGAATGGACCACATGGTTTCTTCCTTTTGCATTGAGAACTG |
| | TCATCGGGTGGAGTCACTGTCCCTGGGGTTTCTCCATAACATGCCCAAGGAGGAAG |
| | AGGAGGAGGAAAAGGAAGGCCGACACCTTGATATGGTGCAGTGTGTCCTCCCAAGC |
| | TCCTCTCATGCTGCCTGTTCTCATGGATTGGTGAACAGCCACCTCACTTCCAGTTTTT |
| | GCCGGGGCCTCTTTTCAGTTCTGAGCACCAGCCAGAGTCTAACTGAATTGGACCTCA |
| | GTGACAATTCTCTGGGGGACCCAGGGATGAGAGTGTTGTGTGAAACGCTCCAGCAT |
| | CCTGGCTGTAACATTCGGAGATTGTGGTTGGGGCGCTGTGGCCTCTCGCATGAGTGC |
| | TGCTTCGACATCTCCTTGGTCCTCAGCAGCAACCAGAAGCTGGTGGAGCTGGACCTG |
| | AGTGACAACGCCCTCGGTGACTTCGGAATCAGACTTCTGTGTGTGGGACTGAAGCA |
| | CCTGTTGTGCAATCTGAAGAAGCTCTGGTTGGTCAGCTGCTGCCTCACATCAGCATG |
| | TTGTCAGGATCTTGCATCAGTATTGAGCACCAGCCATTCCCTGACCAGACTCTATGT |
| | GGGGGAGAATGCCTTGGGAGACTCAGGAGTCGCAATTTTATGTGAAAAAGCCAAGA |
| | ATCCACAGTGTAACCTGCAGAAACTGGGGTTGGTGAATTCTGGCCTTACGTCAGTCT |
| | GTTGTTCAGCTTTGTCCTCGGTACTCAGCACTAATCAGAATCTCACGCACCTTTACCT |
| | GCGAGGCAACACTCTCGGAGACAAGGGGATCAAACTACTCTGTGAGGGACTCTTGC |
| | ACCCCGACTGCAAGCTTCAGGTGTTGGAATTAGACAACTGCAACCTCACGTCACACT |
| | GCTGCTGGGATCTTTCCACACTTCTGACCTCCAGCCAGAGCCTGCGAAAGCTGAGCC |
| | TGGGCAACAATGACCTGGGCGACCTGGGGGTCATGATGTTCTGTGAAGTGCTGAAA |
| | CAGCAGAGCTGCCTCCTGCAGAACCTGGGGTTGTCTGAAATGTATTTCAATTATGAG |
| | ACAAAAAGTGCGTTAGAAACACTTCAAGAAGAAAAGCCTGAGCTGACCGTCGTCTT |
| | TGAGCCTTCTTGGTAGGAGTGGAAACGGGGCTGCCAGACGCCAGTGTTCTCCGGTCC |
| | CTCCAGCTGGGGGCCCTCAGGTGGAGAGAGCTGCGATCCATCCAGGCCAAGACCAC |
| | AGCTCTGTGATCCTTCCGGTGGAGTGTCGGAGAAGAGAGCTTGCCGACGATGCCTTC |
| | CTGTGCAGAGCTTGGGCATCTCCTTTACGCCAGGGTGAGGAAGACACCAGGACAAT |
| | GACAGCATCGGGTGTTGTTGTCATCACAGCGCCTCAGTTAGAGGATGTTCCTCTTGG |
| | TGACCTCATGTAATTAGCTCATTCAATAAAGCACTTTCTTTATTTT |
| 57 | GTTCCTGAGGCTGGCATCTGGATGAGGAAACTGAAGTTGAGGAATAGTGAAGAGTT |
| | TGTCCAATGTCATAGCCCCGTAATCAACGGGACAAAAATTTTCTTGCTGATGGGTCA |
| | AGATGGCATCGTGAAGTGGTTGTTCACCGTAAACTGTAATACAATCCTGTTTATGGA |
| | TTTGTTTGCATATTTTTCCCTCCATAGGGAAACCTTTCTTCCATGGCTCAGGACACAC |
| | TCCTGGATCGAGCCAACAGGAGAACTTTCTGGTAAGCATTTGGCTAACTTTTTTTTTT |
| | TTGAGATGGAGTCTTGCTGTGTCGCCTAGGCTGGAGTGCAGTGGCGTGATCTTGGCT |
| | CACTGCAGCCTCCACTTCCCGGGTTCAATCAATTCTCCTACCTCAACTTCCTGAGTAG |
| | CTGGGATTACAGGCGCCCGCCACCACACCCGGCTCATTTTTGTACTTTTAGTAGAGA |
| | CACAGTTTTGCCATGTTGGCCAGGCTGGTCTTGAATTCCTCAGCTCAGGTGATCTGC |
| | CTGCCTTGGCCTCTCAAAGTGCTGGGATTACAGGCGTGAGCCACTGTGCCCGGCCTT |
| | GGCTAACTTTTCAAAATTAAAGATTTTGACTTGTTACAGTCATGTGACATTTTTTTCT |
| | TTCTGTTTGCTGAGTTTTTGATAATTTATATCTCTCAAAGTGGAGACTTTAAAAAAGA |
| | CTCATCCGTGTGCCGTGTTCACTGCCTGGTATCTTAGTGTGGACCGAAGCCTAAGGA |
| | CCCTGAAAACAGCTGCAGATGAAGATGGCAAGCACCCGCTGCAAGCTGGCCAGGTA |
| | CCTGGAGGACCTGGAGGATGTGGACTTGAAGAAATTTAAGATGCACTTAGAGGACT |
| | ATCCTCCCCAGAAGGGCTGCATCCCCCTCCCGAGGGGTCAGACAGAGAAGGCAGAC |
| | CATGTGGATCTAGCCACGCTAATGATCGACTTCAATGGGGAGGAGAAGGCGTGGGC |
| | CATGGCCGTGTGGATCTTCGCTGCGATCAACAGGAGAGACCTTTATGAGAAAGCAA |
| | AAAGAGATGAGCCGAAGTGGGGTTCAGATAATGCACGTGTTTCGAATCCCACTGTG |
| | ATATGCCAGGAAGACAGCATTGAAGAGGAGTGGATGGGTTTACTGGAGTACCTTTC |
| | GAGAATCTCTATTTGTAAAATGAAGAAAGATTACCGTAAGAAGTACAGAAAGTACG |
| | TGAGAAGCAGATTCCAGTGCATTGAAGACAGGAATGCCCGTCTGGGTGAGAGTGTG |
| | AGCCTCAACAAACGCTACACACGACTGCGTCTCATCAAGGAGCACCGGAGCCAGCA |
| | GGAGAGGGAGCAGGAGCTTCTGGCCATCGGCAAGACCAAGACGTGTGAGAGCCCC |
| | GTGAGTCCCATTAAGATGGAGTTGCTGTTTGACCCCGATGATGAGCATTCTGAGCCT |
| | GTGCACACCGTGGTGTTCCAGGGGGCGGCAGGGATTGGGAAAACAATCCTGGCCAG |
| | GAAGATGATGTTGGACTGGGCGTCGGGGACACTCTACCAAGACAGGTTTGACTATC |
| | TGTTCTATATCCACTGTCGAGAGGTGAGCCTTGTGACACAGAGGAGCCTGGGGGAC |
| | CTGATCATGAGCTGCTGCCCCGACCCAAACCCACCCATCCACAAGATCGTGAGAAA |
| | ACCCTCCAGAATCCTCTTCCTCATGGACGGCTTCGATGAGCTGCAAGGTGCCTTTGA |
| | CGAGCACATAGGACCGCTCTGCACTGACTGGCAGAAGGCCGAGCGGGGAGACATTC |
| | TCCTGAGCAGCCTCATCAGAAAGAAGCTGCTTCCCGAGGCCTCTCTGCTCATCACCA |
| | CGAGACCTGTGGCCCTGGAGAAACTGCAGCACTTGCTGGACCATCCTCGGCATGTG |
| | GAGATCCTGGGTTTCTCCGAGGCCAAAAGGAAAGAGTACTTCTTCAAGTACTTCTCT |
| | GATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCA |
| | GATGGAGAGTGGCAAGAGCCTTGCCCAGACATCCAAGACCACCACCGCGGTGTACG |
| | TCTTCTTCCTTTCCAGTTTGCTGCAGCCCCGGGGAGGGAGCCAGGAGCACGGCCTCT |
| | GCGCCCACCTCTGGGGGCTCTGCTCTTTGGCTGCAGATGGAATCTGGAACCAGAAA |
| | ATCCTGTTTGAGGAGTCCGACCTCAGGAATCATGGACTGCAGAAGGCGGATGTGTC |
| | TGCTTTCCTGAGGATGAACCTGTTCCAAAAGGAAGTGGACTGCGAGAAGTTCTACA |
| | GCTTCATCCACATGACTTTCCAGGAGTTCTTTGCCGCCATGTACTACCTGCTGGAAG |
| | AGGAAAAGGAAGGAAGGACGAACGTTCCAGGGAGTCGTTTGAAGCTTCCCAGCCG |
| | AGACGTGACAGTCCTTCTGGAAAACTATGGCAAATTCGAAAAGGGGTATTTGATTTT |
| | TGTTGTACGTTTCCTCTTTGGCCTGGTAAACCAGGAGAGGACCTCCTACTTGGAGAA |
| | GAAATTAAGTTGCAAGATCTCTCAGCAAATCAGGCTGGAGCTGCTGAAATGGATTG |
| | AAGTGAAAGCCAAAGCTAAAAAGCTGCAGATCCAGCCCAGCCAGCTGGAATTGTTC |
| | TACTGTTTGTACGAGATGCAGGAGGAGGACTTCGTGCAAAGGGCCATGGACTATTT |
| | CCCCAAGATTGAGATCAATCTCTCCACCAGAATGGACCACATGGTTTCTTCCTTTTG |
| | CATTGAGAACTGTCATCGGGTGGAGTCACTGTCCCTGGGGTTTCTCCATAACATGCC |
| | CAAGGAGGAAGAGGAGGAGGAAAAGGAAGGCCGACACCTTGATATGGTGCAGTGT |
| | GTCCTCCCAAGCTCCTCTCATGCTGCCTGTTCTCATGGATTGGTGAACAGCCACCTC |
| | ACTTCCAGTTTTTGCCGGGGCCTCTTTTCAGTTCTGAGCACCAGCCAGAGTCTAACT |
| | GAATTGGACCTCAGTGACAATTCTCTGGGGGACCCAGGGATGAGAGTGTTGTGTGA |
| | AACGCTCCAGCATCCTGGCTGTAACATTCGGAGATTGTGGTTGGGGCGCTGTGGCCT |
| | CTCGCATGAGTGCTGCTTCGACATCTCCTTGGTCCTCAGCAGCAACCAGAAGCTGGT |
| | GGAGCTGGACCTGAGTGACAACGCCCTCGGTGACTTCGGAATCAGACTTCTGTGTGT |
| | GGGACTGAAGCACCTGTTGTGCAATCTGAAGAAGCTCTGGTTGGTGAATTCTGGCCT |
| | TACGTCAGTCTGTTGTTCAGCTTTGTCCTCGGTACTCAGCACTAATCAGAATCTCACG |
| | CACCTTTACCTGCGAGGCAACACTCTCGGAGACAAGGGGATCAAACTACTCTGTGA |
| | GGGACTCTTGCACCCCGACTGCAAGCTTCAGGTGTTGGAATTAGACAACTGCAACCT |
| | CACGTCACACTGCTGCTGGGATCTTTCCACACTTCTGACCTCCAGCCAGAGCCTGCG |
| | AAAGCTGAGCCTGGGCAACAATGACCTGGGCGACCTGGGGGTCATGATGTTCTGTG |
| | AAGTGCTGAAACAGCAGAGCTGCCTCCTGCAGAACCTGGGGTTGTCTGAAATGTAT |
| | TTCAATTATGAGACAAAAAGTGCGTTAGAAACACTTCAAGAAGAAAAGCCTGAGCT |
| | GACCGTCGTCTTTGAGCCTTCTTGGTAGGAGTGGAAACGGGGCTGCCAGACGCCAG |
| | TGTTCTCCGGTCCCTCCAGCTGGGGGCCCTCAGGTGGAGAGAGCTGCGATCCATCCA |
| | GGCCAAGACCACAGCTCTGTGATCCTTCCGGTGGAGTGTCGGAGAAGAGAGCTTGC |
| | CGACGATGCCTTCCTGTGCAGAGCTTGGGCATCTCCTTTACGCCAGGGTGAGGAAGA |
| | CACCAGGACAATGACAGCATCGGGTGTTGTTGTCATCACAGCGCCTCAGTTAGAGG |
| | ATGTTCCTCTTGGTGACCTCATGTAATTAGCTCATTCAATAAAGCACTTTCTTTATTT |
| | T |
| 58 | GTTCCTGAGGCTGGCATCTGGATGAGGAAACTGAAGTTGAGGAATAGTGAAGAGTT |
| | TGTCCAATGTCATAGCCCCGTAATCAACGGGACAAAAATTTTCTTGCTGATGGGTCA |
| | AGATGGCATCGTGAAGTGGTTGTTCACCGTAAACTGTAATACAATCCTGTTTATGGA |
| | TTTGTTTGCATATTTTTCCCTCCATAGGGAAACCTTTCTTCCATGGCTCAGGACACAC |
| | TCCTGGATCGAGCCAACAGGAGAACTTTCTGGTAAGCATTTGGCTAACTTTTTTTTTT |
| | TTGAGATGGAGTCTTGCTGTGTCGCCTAGGCTGGAGTGCAGTGGCGTGATCTTGGCT |
| | CACTGCAGCCTCCACTTCCCGGGTTCAATCAATTCTCCTACCTCAACTTCCTGAGTAG |
| | CTGGGATTACAGGCGCCCGCCACCACACCCGGCTCATTTTTGTACTTTTAGTAGAGA |
| | CACAGTTTTGCCATGTTGGCCAGGCTGGTCTTGAATTCCTCAGCTCAGGTGATCTGC |
| | CTGCCTTGGCCTCTCAAAGTGCTGGGATTACAGGCGTGAGCCACTGTGCCCGGCCTT |
| | GGCTAACTTTTCAAAATTAAAGATTTTGACTTGTTACAGTCATGTGACATTTTTTTCT |
| | TTCTGTTTGCTGAGTTTTTGATAATTTATATCTCTCAAAGTGGAGACTTTAAAAAAGA |
| | CTCATCCGTGTGCCGTGTTCACTGCCTGGTATCTTAGTGTGGACCGAAGCCTAAGGA |
| | CCCTGAAAACAGCTGCAGATGAAGATGGCAAGCACCCGCTGCAAGCTGGCCAGGTA |
| | CCTGGAGGACCTGGAGGATGTGGACTTGAAGAAATTTAAGATGCACTTAGAGGACT |
| | ATCCTCCCCAGAAGGGCTGCATCCCCCTCCCGAGGGGTCAGACAGAGAAGGCAGAC |
| | CATGTGGATCTAGCCACGCTAATGATCGACTTCAATGGGGAGGAGAAGGCGTGGGC |
| | CATGGCCGTGTGGATCTTCGCTGCGATCAACAGGAGAGACCTTTATGAGAAAGCAA |
| | AAAGAGATGAGCCGAAGTGGGGTTCAGATAATGCACGTGTTTCGAATCCCACTGTG |
| | ATATGCCAGGAAGACAGCATTGAAGAGGAGTGGATGGGTTTACTGGAGTACCTTTC |
| | GAGAATCTCTATTTGTAAAATGAAGAAAGATTACCGTAAGAAGTACAGAAAGTACG |
| | TGAGAAGCAGATTCCAGTGCATTGAAGACAGGAATGCCCGTCTGGGTGAGAGTGTG |
| | AGCCTCAACAAACGCTACACACGACTGCGTCTCATCAAGGAGCACCGGAGCCAGCA |
| | GGAGAGGGAGCAGGAGCTTCTGGCCATCGGCAAGACCAAGACGTGTGAGAGCCCC |
| | GTGAGTCCCATTAAGATGGAGTTGCTGTTTGACCCCGATGATGAGCATTCTGAGCCT |
| | GTGCACACCGTGGTGTTCCAGGGGGCGGCAGGGATTGGGAAAACAATCCTGGCCAG |
| | GAAGATGATGTTGGACTGGGCGTCGGGGACACTCTACCAAGACAGGTTTGACTATC |
| | TGTTCTATATCCACTGTCGAGAGGTGAGCCTTGTGACACAGAGGAGCCTGGGGGAC |
| | CTGATCATGAGCTGCTGCCCCGACCCAAACCCACCCATCCACAAGATCGTGAGAAA |
| | ACCCTCCAGAATCCTCTTCCTCATGGACGGCTTCGATGAGCTGCAAGGTGCCTTTGA |
| | CGAGCACATAGGACCGCTCTGCACTGACTGGCAGAAGGCCGAGCGGGGAGACATTC |
| | TCCTGAGCAGCCTCATCAGAAAGAAGCTGCTTCCCGAGGCCTCTCTGCTCATCACCA |
| | CGAGACCTGTGGCCCTGGAGAAACTGCAGCACTTGCTGGACCATCCTCGGCATGTG |
| | GAGATCCTGGGTTTCTCCGAGGCCAAAAGGAAAGAGTACTTCTTCAAGTACTTCTCT |
| | GATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCA |
| | GATGGAGAGTGGCAAGAGCCTTGCCCAGACATCCAAGACCACCACCGCGGTGTACG |
| | TCTTCTTCCTTTCCAGTTTGCTGCAGCCCCGGGGAGGGAGCCAGGAGCACGGCCTCT |
| | GCGCCCACCTCTGGGGGCTCTGCTCTTTGGCTGCAGATGGAATCTGGAACCAGAAA |
| | ATCCTGTTTGAGGAGTCCGACCTCAGGAATCATGGACTGCAGAAGGCGGATGTGTC |
| | TGCTTTCCTGAGGATGAACCTGTTCCAAAAGGAAGTGGACTGCGAGAAGTTCTACA |
| | GCTTCATCCACATGACTTTCCAGGAGTTCTTTGCCGCCATGTACTACCTGCTGGAAG |
| | AGGAAAAGGAAGGAAGGACGAACGTTCCAGGGAGTCGTTTGAAGCTTCCCAGCCG |
| | AGACGTGACAGTCCTTCTGGAAAACTATGGCAAATTCGAAAAGGGGTATTTGATTTT |
| | TGTTGTACGTTTCCTCTTTGGCCTGGTAAACCAGGAGAGGACCTCCTACTTGGAGAA |
| | GAAATTAAGTTGCAAGATCTCTCAGCAAATCAGGCTGGAGCTGCTGAAATGGATTG |
| | AAGTGAAAGCCAAAGCTAAAAAGCTGCAGATCCAGCCCAGCCAGCTGGAATTGTTC |
| | TACTGTTTGTACGAGATGCAGGAGGAGGACTTCGTGCAAAGGGCCATGGACTATTT |
| | CCCCAAGATTGAGATCAATCTCTCCACCAGAATGGACCACATGGTTTCTTCCTTTTG |
| | CATTGAGAACTGTCATCGGGTGGAGTCACTGTCCCTGGGGTTTCTCCATAACATGCC |
| | CAAGGAGGAAGAGGAGGAGGAAAAGGAAGGCCGACACCTTGATATGGTGCAGTGT |
| | GTCCTCCCAAGCTCCTCTCATGCTGCCTGTTCTCATGGGTTGGGGCGCTGTGGCCTCT |
| | CGCATGAGTGCTGCTTCGACATCTCCTTGGTCCTCAGCAGCAACCAGAAGCTGGTGG |
| | AGCTGGACCTGAGTGACAACGCCCTCGGTGACTTCGGAATCAGACTTCTGTGTGTGG |
| | GACTGAAGCACCTGTTGTGCAATCTGAAGAAGCTCTGGTTGGTCAGCTGCTGCCTCA |
| | CATCAGCATGTTGTCAGGATCTTGCATCAGTATTGAGCACCAGCCATTCCCTGACCA |
| | GACTCTATGTGGGGGAGAATGCCTTGGGAGACTCAGGAGTCGCAATTTTATGTGAA |
| | AAAGCCAAGAATCCACAGTGTAACCTGCAGAAACTGGGGTTGGTGAATTCTGGCCT |
| | TACGTCAGTCTGTTGTTCAGCTTTGTCCTCGGTACTCAGCACTAATCAGAATCTCACG |
| | CACCTTTACCTGCGAGGCAACACTCTCGGAGACAAGGGGATCAAACTACTCTGTGA |
| | GGGACTCTTGCACCCCGACTGCAAGCTTCAGGTGTTGGAATTAGACAACTGCAACCT |
| | CACGTCACACTGCTGCTGGGATCTTTCCACACTTCTGACCTCCAGCCAGAGCCTGCG |
| | AAAGCTGAGCCTGGGCAACAATGACCTGGGCGACCTGGGGGTCATGATGTTCTGTG |
| | AAGTGCTGAAACAGCAGAGCTGCCTCCTGCAGAACCTGGGGTTGTCTGAAATGTAT |
| | TTCAATTATGAGACAAAAAGTGCGTTAGAAACACTTCAAGAAGAAAAGCCTGAGCT |
| | GACCGTCGTCTTTGAGCCTTCTTGGTAGGAGTGGAAACGGGGCTGCCAGACGCCAG |
| | TGTTCTCCGGTCCCTCCAGCTGGGGGCCCTCAGGTGGAGAGAGCTGCGATCCATCCA |
| | GGCCAAGACCACAGCTCTGTGATCCTTCCGGTGGAGTGTCGGAGAAGAGAGCTTGC |
| | CGACGATGCCTTCCTGTGCAGAGCTTGGGCATCTCCTTTACGCCAGGGTGAGGAAGA |
| | CACCAGGACAATGACAGCATCGGGTGTTGTTGTCATCACAGCGCCTCAGTTAGAGG |
| | ATGTTCCTCTTGGTGACCTCATGTAATTAGCTCATTCAATAAAGCACTTTCTTTATTT |
| | T |
| 59 | GTTCCTGAGGCTGGCATCTGGATGAGGAAACTGAAGTTGAGGAATAGTGAAGAGTT |
| | TGTCCAATGTCATAGCCCCGTAATCAACGGGACAAAAATTTTCTTGCTGATGGGTCA |
| | AGATGGCATCGTGAAGTGGTTGTTCACCGTAAACTGTAATACAATCCTGTTTATGGA |
| | TTTGTTTGCATATTTTTCCCTCCATAGGGAAACCTTTCTTCCATGGCTCAGGACACAC |
| | TCCTGGATCGAGCCAACAGGAGAACTTTCTGGTAAGCATTTGGCTAACTTTTTTTTTT |
| | TTGAGATGGAGTCTTGCTGTGTCGCCTAGGCTGGAGTGCAGTGGCGTGATCTTGGCT |
| | CACTGCAGCCTCCACTTCCCGGGTTCAATCAATTCTCCTACCTCAACTTCCTGAGTAG |
| | CTGGGATTACAGGCGCCCGCCACCACACCCGGCTCATTTTTGTACTTTTAGTAGAGA |
| | CACAGTTTTGCCATGTTGGCCAGGCTGGTCTTGAATTCCTCAGCTCAGGTGATCTGC |
| | CTGCCTTGGCCTCTCAAAGTGCTGGGATTACAGGCGTGAGCCACTGTGCCCGGCCTT |
| | GGCTAACTTTTCAAAATTAAAGATTTTGACTTGTTACAGTCATGTGACATTTTTTTCT |
| | TTCTGTTTGCTGAGTTTTTGATAATTTATATCTCTCAAAGTGGAGACTTTAAAAAAGA |
| | CTCATCCGTGTGCCGTGTTCACTGCCTGGTATCTTAGTGTGGACCGAAGCCTAAGGA |
| | CCCTGAAAACAGCTGCAGATGAAGATGGCAAGCACCCGCTGCAAGCTGGCCAGGTA |
| | CCTGGAGGACCTGGAGGATGTGGACTTGAAGAAATTTAAGATGCACTTAGAGGACT |
| | ATCCTCCCCAGAAGGGCTGCATCCCCCTCCCGAGGGGTCAGACAGAGAAGGCAGAC |
| | CATGTGGATCTAGCCACGCTAATGATCGACTTCAATGGGGAGGAGAAGGCGTGGGC |
| | CATGGCCGTGTGGATCTTCGCTGCGATCAACAGGAGAGACCTTTATGAGAAAGCAA |
| | AAAGAGATGAGCCGAAGTGGGGTTCAGATAATGCACGTGTTTCGAATCCCACTGTG |
| | ATATGCCAGGAAGACAGCATTGAAGAGGAGTGGATGGGTTTACTGGAGTACCTTTC |
| | GAGAATCTCTATTTGTAAAATGAAGAAAGATTACCGTAAGAAGTACAGAAAGTACG |
| | TGAGAAGCAGATTCCAGTGCATTGAAGACAGGAATGCCCGTCTGGGTGAGAGTGTG |
| | AGCCTCAACAAACGCTACACACGACTGCGTCTCATCAAGGAGCACCGGAGCCAGCA |
| | GGAGAGGGAGCAGGAGCTTCTGGCCATCGGCAAGACCAAGACGTGTGAGAGCCCC |
| | GTGAGTCCCATTAAGATGGAGTTGCTGTTTGACCCCGATGATGAGCATTCTGAGCCT |
| | GTGCACACCGTGGTGTTCCAGGGGGCGGCAGGGATTGGGAAAACAATCCTGGCCAG |
| | GAAGATGATGTTGGACTGGGCGTCGGGGACACTCTACCAAGACAGGTTTGACTATC |
| | TGTTCTATATCCACTGTCGAGAGGTGAGCCTTGTGACACAGAGGAGCCTGGGGGAC |
| | CTGATCATGAGCTGCTGCCCCGACCCAAACCCACCCATCCACAAGATCGTGAGAAA |
| | ACCCTCCAGAATCCTCTTCCTCATGGACGGCTTCGATGAGCTGCAAGGTGCCTTTGA |
| | CGAGCACATAGGACCGCTCTGCACTGACTGGCAGAAGGCCGAGCGGGGAGACATTC |
| | TCCTGAGCAGCCTCATCAGAAAGAAGCTGCTTCCCGAGGCCTCTCTGCTCATCACCA |
| | CGAGACCTGTGGCCCTGGAGAAACTGCAGCACTTGCTGGACCATCCTCGGCATGTG |
| | GAGATCCTGGGTTTCTCCGAGGCCAAAAGGAAAGAGTACTTCTTCAAGTACTTCTCT |
| | GATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCA |
| | GATGGAGAGTGGCAAGAGCCTTGCCCAGACATCCAAGACCACCACCGCGGTGTACG |
| | TCTTCTTCCTTTCCAGTTTGCTGCAGCCCCGGGGAGGGAGCCAGGAGCACGGCCTCT |
| | GCGCCCACCTCTGGGGGCTCTGCTCTTTGGCTGCAGATGGAATCTGGAACCAGAAA |
| | ATCCTGTTTGAGGAGTCCGACCTCAGGAATCATGGACTGCAGAAGGCGGATGTGTC |
| | TGCTTTCCTGAGGATGAACCTGTTCCAAAAGGAAGTGGACTGCGAGAAGTTCTACA |
| | GCTTCATCCACATGACTTTCCAGGAGTTCTTTGCCGCCATGTACTACCTGCTGGAAG |
| | AGGAAAAGGAAGGAAGGACGAACGTTCCAGGGAGTCGTTTGAAGCTTCCCAGCCG |
| | AGACGTGACAGTCCTTCTGGAAAACTATGGCAAATTCGAAAAGGGGTATTTGATTTT |
| | TGTTGTACGTTTCCTCTTTGGCCTGGTAAACCAGGAGAGGACCTCCTACTTGGAGAA |
| | GAAATTAAGTTGCAAGATCTCTCAGCAAATCAGGCTGGAGCTGCTGAAATGGATTG |
| | AAGTGAAAGCCAAAGCTAAAAAGCTGCAGATCCAGCCCAGCCAGCTGGAATTGTTC |
| | TACTGTTTGTACGAGATGCAGGAGGAGGACTTCGTGCAAAGGGCCATGGACTATTT |
| | CCCCAAGATTGAGATCAATCTCTCCACCAGAATGGACCACATGGTTTCTTCCTTTTG |
| | CATTGAGAACTGTCATCGGGTGGAGTCACTGTCCCTGGGGTTTCTCCATAACATGCC |
| | CAAGGAGGAAGAGGAGGAGGAAAAGGAAGGCCGACACCTTGATATGGTGCAGTGT |
| | GTCCTCCCAAGCTCCTCTCATGCTGCCTGTTCTCATGGATTGGTGAACAGCCACCTC |
| | ACTTCCAGTTTTTGCCGGGGCCTCTTTTCAGTTCTGAGCACCAGCCAGAGTCTAACT |
| | GAATTGGACCTCAGTGACAATTCTCTGGGGGACCCAGGGATGAGAGTGTTGTGTGA |
| | AACGCTCCAGCATCCTGGCTGTAACATTCGGAGATTGTGGTTGGGGCGCTGTGGCCT |
| | CTCGCATGAGTGCTGCTTCGACATCTCCTTGGTCCTCAGCAGCAACCAGAAGCTGGT |
| | GGAGCTGGACCTGAGTGACAACGCCCTCGGTGACTTCGGAATCAGACTTCTGTGTGT |
| | GGGACTGAAGCACCTGTTGTGCAATCTGAAGAAGCTCTGGTTGGTCAGCTGCTGCCT |
| | CACATCAGCATGTTGTCAGGATCTTGCATCAGTATTGAGCACCAGCCATTCCCTGAC |
| | CAGACTCTATGTGGGGGAGAATGCCTTGGGAGACTCAGGAGTCGCAATTTTATGTG |
| | AAAAAGCCAAGAATCCACAGTGTAACCTGCAGAAACTGGGGTTGGTGAATTCTGGC |
| | CTTACGTCAGTCTGTTGTTCAGCTTTGTCCTCGGTACTCAGCACTAATCAGAATCTCA |
| | CGCACCTTTACCTGCGAGGCAACACTCTCGGAGACAAGGGGATCAAACTACTCTGT |
| | GAGGGACTCTTGCACCCCGACTGCAAGCTTCAGGTGTTGGAATTAGACAACTGCAA |
| | CCTCACGTCACACTGCTGCTGGGATCTTTCCACACTTCTGACCTCCAGCCAGAGCCT |
| | GCGAAAGCTGAGCCTGGGCAACAATGACCTGGGCGACCTGGGGGTCATGATGTTCT |
| | GTGAAGTGCTGAAACAGCAGAGCTGCCTCCTGCAGAACCTGGGGTTGTCTGAAATG |
| | TATTTCAATTATGAGACAAAAAGTGCGTTAGAAACACTTCAAGAAGAAAAGCCTGA |
| | GCTGACCGTCGTCTTTGAGCCTTCTTGGTAGGAGTGGAAACGGGGCTGCCAGACGCC |
| | AGTGTTCTCCGGTCCCTCCAGCTGGGGGCCCTCAGGTGGAGAGAGCTGCGATCCATC |
| | CAGGCCAAGACCACAGCTCTGTGATCCTTCCGGTGGAGTGTCGGAGAAGAGAGCTT |
| | GCCGACGATGCCTTCCTGTGCAGAGCTTGGGCATCTCCTTTACGCCAGGGTGAGGAA |
| | GACACCAGGACAATGACAGCATCGGGTGTTGTTGTCATCACAGCGCCTCAGTTAGA |
| | GGATGTTCCTCTTGGTGACCTCATGTAATTAGCTCATTCAATAAAGCACTTTCTTTAT |
| | TTT |
| 60 | GTTGACTAGGCGCTGTTCTTGCTGGCTGGTGCCCCAGGGCCTGGAGAGGTCTGAAGA |
| | AACCTGGGAGCCAGCAGCCCGGGGCTCCACTCTGGGTTCTGAAAGCCCATTCCCTGC |
| | TCTGCGGCTCCTCCCACCCCACCTCTTCTCAGCCTTGCAGCTCAAGGGTTGATCTCAG |
| | GAGTCCAGGACCCAGGAGAGGGAAGAATCTGAGGAACACAGAACAGTGAGCGTTG |
| | CCCACACCCCATCTCCCGTCACCACATCTCCCCTCACCCTCACCCTCCCTGCCTGGCC |
| | CTGGACCCCATCCCAGGACCTCCCTATCAGCTGACTTCTTCCAGTGTCTTGCAGGCC |
| | CCTCTGGGCTCCTCCCTCCCCTGGCTTTTCCTACCACTCCCCCTCTATCGGCGTCTAT |
| | CTGTAGGTGCCCTGGGATTTATAAAACTGGGTTCCGAATGCTGAATAAGAGACGGT |
| | AAGAGCCAAGGCAAAGGACAGCACTGTTCTCTGCCTGCCTGATACCCTCACCACCT |
| | GGGAACATCCCCCAGACACCCTCTTAACTCCGGGACAGAGATGGCTGGCGGAGCCT |
| | GGGGCCGCCTGGCCTGTTACTTGGAGTTCCTGAAGAAGGAGGAGCTGAAGGAGTTC |
| | CAGCTTCTGCTCGCCAATAAAGCGCACTCCAGGAGCTCTTCGGGTGAGACACCCGCT |
| | CAGCCAGAGAAGACGAGTGGCATGGAGGTGGCCTCGTACCTGGTGGCTCAGTATGG |
| | GGAGCAGCGGGCCTGGGACCTAGCCCTCCATACCTGGGAGCAGATGGGGCTGAGGT |
| | CACTGTGCGCCCAAGCCCAGGAAGGGGCAGGCCACTCTCCCTCATTCCCCTACAGCC |
| | CAAGTGAACCCCACCTGGGGTCTCCCAGCCAACCCACCTCCACCGCAGTGCTAATGC |
| | CCTGGATCCATGAATTGCCGGCGGGGTGCACCCAGGGCTCAGAGAGAAGGGTTTTG |
| | AGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAAC |
| | GCCCCCACATCCACAGCAGTGCTGGGGAGCTGGGGATCCCCACCTCAGCCCAGCCT |
| | AGCACCCAGAGAGCAGGAGGCTCCTGGGACCCAATGGCCTCTGGATGAAACGTCAG |
| | GAATTTACTACACAGAAATCAGAGAAAGAGAGAGAGAGAAATCAGAGAAAGGCAG |
| | GCCCCCATGGGCAGCGGTGGTAGGAACGCCCCCACAGGCGCACACCAGCCTACAGC |
| | CCCACCACCACCCATGGGAGCCTTCTGTGAGAGAGAGCCTCTGTTCCACATGGCCCT |
| | GGAAAAATGAGGATTTTAACCAAAAATTCACACAGCTGCTACTTCTACAAAGACCT |
| | CACCCCAGAAGCCAAGATCCCCTGGTCAAGAGAAGCTGGCCTGATTATGTGGAGGA |
| | GAATCGAGGACATTTAATTGAGATCAGAGACTTATTTGGCCCAGGCCTGGATACCC |
| | AAGAACCTCGCATAGTCATACTGCAGGGGGCTGCTGGAATTGGGAAGTCAACACTG |
| | GCCAGGCAGGTGAAGGAAGCCTGGGGGAGAGGCCAGCTGTATGGGGACCGCTTCC |
| | AGCATGTCTTCTACTTCAGCTGCAGAGAGCTGGCCCAGTCCAAGGTGGTGAGTCTCG |
| | CTGAGCTCATCGGAAAAGATGGGACAGCCACTCCGGCTCCCATTAGACAGATCCTG |
| | TCTAGGCCAGAGCGGCTGCTCTTCATCCTCGATGGTGTAGATGAGCCAGGATGGGTC |
| | TTGCAGGAGCCGAGTTCTGAGCTCTGTCTGCACTGGAGCCAGCCACAGCCGGCGGA |
| | TGCACTGCTGGGCAGTTTGCTGGGGAAAACTATACTTCCCGAGGCATCCTTCCTGAT |
| | CACGGCTCGGACCACAGCTCTGCAGAACCTCATTCCTTCTTTGGAGCAGGCACGTTG |
| | GGTAGAGGTCCTGGGGTTCTCTGAGTCCAGCAGGAAGGAATATTTCTACAGATATTT |
| | CACAGATGAAAGGCAAGCAATTAGAGCCTTTAGGTTGGTCAAATCAAACAAAGAGC |
| | TCTGGGCCCTGTGTCTTGTGCCCTGGGTGTCCTGGCTGGCCTGCACTTGCCTGATGCA |
| | GCAGATGAAGCGGAAGGAAAAACTCACACTGACTTCCAAGACCACCACAACCCTCT |
| | GTCTACATTACCTTGCCCAGGCTCTCCAAGCTCAGCCATTGGGACCCCAGCTCAGAG |
| | ACCTCTGCTCTCTGGCTGCTGAGGGCATCTGGCAAAAAAAGACCCTTTTCAGTCCAG |
| | ATGACCTCAGGAAGCATGGGTTAGATGGGGCCATCATCTCCACCTTCTTGAAGATGG |
| | GTATTCTTCAAGAGCACCCCATCCCTCTGAGCTACAGCTTCATTCACCTCTGTTTCCA |
| | AGAGTTCTTTGCAGCAATGTCCTATGTCTTGGAGGATGAGAAGGGGAGAGGTAAAC |
| | ATTCTAATTGCATCATAGATTTGGAAAAGACGCTAGAAGCATATGGAATACATGGC |
| | CTGTTTGGGGCATCAACCACACGTTTCCTATTGGGCCTGTTAAGTGATGAGGGGGAG |
| | AGAGAGATGGAGAACATCTTTCACTGCCGGCTGTCTCAGGGGAGGAACCTGATGCA |
| | GTGGGTCCCGTCCCTGCAGCTGCTGCTGCAGCCACACTCTCTGGAGTCCCTCCACTG |
| | CTTGTACGAGACTCGGAACAAAACGTTCCTGACACAAGTGATGGCCCATTTCGAAG |
| | AAATGGGCATGTGTGTAGAAACAGACATGGAGCTCTTAGTGTGCACTTTCTGCATTA |
| | AATTCAGCCGCCACGTGAAGAAGCTTCAGCTGATTGAGGGCAGGCAGCACAGATCA |
| | ACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTA |
| | AGTGGAAACTCGCTGAGCCACTCTGCAGTGAAGAGTCTTTGTAAGACCCTGAGACG |
| | CCCTCGCTGCCTCCTGGAGACCCTGCGGTTGGCTGGCTGTGGCCTCACAGCTGAGGA |
| | CTGCAAGGACCTTGCCTTTGGGCTGAGAGCCAACCAGACCCTGACCGAGCTGGACC |
| | TGAGCTTCAATGTGCTCACGGATGCTGGAGCCAAACACCTTTGCCAGAGACTGAGA |
| | CAGCCGAGCTGCAAGCTACAGCGACTGCAGCTGGTCAGCTGTGGCCTCACGTCTGA |
| | CTGCTGCCAGGACCTGGCCTCTGTGCTTAGTGCCAGCCCCAGCCTGAAGGAGCTAGA |
| | CCTGCAGCAGAACAACCTGGATGACGTTGGCGTGCGACTGCTCTGTGAGGGGCTCA |
| | GGCATCCTGCCTGCAAACTCATACGCCTGGGGCTGGACCAGACAACTCTGAGTGAT |
| | GAGATGAGGCAGGAACTGAGGGCCCTGGAGCAGGAGAAACCTCAGCTGCTCATCTT |
| | CAGCAGACGGAAACCAAGTGTGATGACCCCTACTGAGGGCCTGGATACGGGAGAG |
| | ATGAGTAATAGCACATCCTCACTCAAGCGGCAGAGACTCGGATCAGAGAGGGCGGC |
| | TTCCCATGTTGCTCAGGCTAATCTCAAACTCCTGGACGTGAGCAAGATCTTCCCAAT |
| | TGCTGAGATTGCAGAGGAAAGCTCCCCAGAGGTAGTACCGGTGGAACTCTTGTGCG |
| | TGCCTTCTCCTGCCTCTCAAGGGGACCTGCATACGAAGCCTTTGGGGACTGACGATG |
| | ACTTCTGGGGCCCCACGGGGCCTGTGGCTACTGAGGTAGTTGACAAAGAAAAGAAC |
| | TTGTACCGAGTTCACTTCCCTGTAGCTGGCTCCTACCGCTGGCCCAACACGGGTCTC |
| | TGCTTTGTGATGAGAGAAGCGGTGACCGTTGAGATTGAATTCTGTGTGTGGGACCAG |
| | TTCCTGGGTGAGATCAACCCACAGCACAGCTGGATGGTGGCAGGGCCTCTGCTGGA |
| | CATCAAGGCTGAGCCTGGAGCTGTGGAAGCTGTGCACCTCCCTCACTTTGTGGCTCT |
| | CCAAGGGGGCCATGTGGACACATCCCTGTTCCAAATGGCCCACTTTAAAGAGGAGG |
| | GGATGCTCCTGGAGAAGCCAGCCAGGGTGGAGCTGCATCACATAGTTCTGGAAAAC |
| | CCCAGCTTCTCCCCCTTGGGAGTCCTCCTGAAAATGATCCATAATGCCCTGCGCTTC |
| | ATTCCCGTCACCTCTGTGGTGTTGCTTTACCACCGCGTCCATCCTGAGGAAGTCACCT |
| | TCCACCTCTACCTGATCCCAAGTGACTGCTCCATTCGGAAGGCCATAGATGATCTAG |
| | AAATGAAATTCCAGTTTGTGCGAATCCACAAGCCACCCCCGCTGACCCCACTTTATA |
| | TGGGCTGTCGTTACACTGTGTCTGGGTCTGGTTCAGGGATGCTGGAAATACTCCCCA |
| | AGGAACTGGAGCTCTGCTATCGAAGCCCTGGAGAAGACCAGCTGTTCTCGGAGTTC |
| | TACGTTGGCCACTTGGGATCAGGGATCAGGCTGCAAGTGAAAGACAAGAAAGATGA |
| | GACTCTGGTGTGGGAGGCCTTGGTGAAACCAGGAGATCTCATGCCTGCAACTACTCT |
| | GATCCCTCCAGCCCGCATAGCCGTACCTTCACCTCTGGATGCCCCGCAGTTGCTGCA |
| | CTTTGTGGACCAGTATCGAGAGCAGCTGATAGCCCGAGTGACATCGGTGGAGGTTG |
| | TCTTGGACAAACTGCATGGACAGGTGCTGAGCCAGGAGCAGTACGAGAGGGTGCTG |
| | GCTGAGAACACGAGGCCCAGCCAGATGCGGAAGCTGTTCAGCTTGAGCCAGTCCTG |
| | GGACCGGAAGTGCAAAGATGGACTCTACCAAGCCCTGAAGGAGACCCATCCTCACC |
| | TCATTATGGAACTCTGGGAGAAGGGCAGCAAAAAGGGACTCCTGCCACTCAGCAGC |
| | TGAAGTATCAACACCAGCCCTTGACCCTTGAGTCCTGGCTTTGGCTGACCCTTCTTTG |
| | GGTCTCAGTTTCTTTCTCTGCAAACAAGTTGCCATCTGGTTTGCCTTCCAGCACTAAA |
| | GTAATGGAACTTTGATGATGCCTTTGCTGGGCATTATGTGTCCATGCCAGGGATGCC |
| | ACAGGGGGCCCCAGTCCAGGTGGCCTAACAGCATCTCAGGGAATGTCCATCTGGAG |
| | CTGGCAAGACCCCTGCAGACCTCATAGAGCCTCATCTGGTGGCCACAGCAGCCAAG |
| | CCTAGAGCCCTCCGGATCCCATCCAGGCGCAAAGAGGAATAGGAGGGACATGGAAC |
| | CATTTGCCTCTGGCTGTGTCACAGGGTGAGCCCCAAAATTGGGGTTCAGCGTGGGAG |
| | GCCACGTGGATTCTTGGCTTTGTACAGGAAGATCTACAAGAGCAAGCCAACAGAGT |
| | AAAGTGGAAGGAAGTTTATTCAGAAAATAAAGGAGTATCACAGCTCTTTTAGAATT |
| | TGTCTAGCAGGCTTTCCAGTTTTTACCAGAAAACCCCTATAAATTAAAAATTTTTTAC |
| | TTAAATTTAAGAATTAAAAAAATACAAAAAAGAAAAAATGAAAATAAAGGAATAA |
| | GAAGTTACCTACTCCA |
| 61 | GTTGACTAGGCGCTGTTCTTGCTGGCTGGTGCCCCAGGGCCTGGAGAGGTCTGAAGA |
| | AACCTGGGAGCCAGCAGCCCGGGGCTCCACTCTGGGTTCTGAAAGCCCATTCCCTGC |
| | TCTGCGGCTCCTCCCACCCCACCTCTTCTCAGCCTTGCAGCTCAAGGGTTGATCTCAG |
| | GAGTCCAGGACCCAGGAGAGGGAAGAATCTGAGGAACACAGAACAGTGAGCGTTG |
| | CCCACACCCCATCTCCCGTCACCACATCTCCCCTCACCCTCACCCTCCCTGCCTGGCC |
| | CTGGACCCCATCCCAGGACCTCCCTATCAGCTGACTTCTTCCAGTGTCTTGCAGGCC |
| | CCTCTGGGCTCCTCCCTCCCCTGGCTTTTCCTACCACTCCCCCTCTATCGGCGTCTAT |
| | CTGTAGGTGCCCTGGGATTTATAAAACTGGGTTCCGAATGCTGAATAAGAGACGGT |
| | AAGAGCCAAGGCAAAGGACAGCACTGTTCTCTGCCTGCCTGATACCCTCACCACCT |
| | GGGAACATCCCCCAGACACCCTCTTAACTCCGGGACAGAGATGGCTGGCGGAGCCT |
| | GGGGCCGCCTGGCCTGTTACTTGGAGTTCCTGAAGAAGGAGGAGCTGAAGGAGTTC |
| | CAGCTTCTGCTCGCCAATAAAGCGCACTCCAGGAGCTCTTCGGGTGAGACACCCGCT |
| | CAGCCAGAGAAGACGAGTGGCATGGAGGTGGCCTCGTACCTGGTGGCTCAGTATGG |
| | GGAGCAGCGGGCCTGGGACCTAGCCCTCCATACCTGGGAGCAGATGGGGCTGAGGT |
| | CACTGTGCGCCCAAGCCCAGGAAGGGGCAGGCCACTCTCCCTCATTCCCCTACAGCC |
| | CAAGTGAACCCCACCTGGGGTCTCCCAGCCAACCCACCTCCACCGCAGTGCTAATGC |
| | CCTGGATCCATGAATTGCCGGCGGGGTGCACCCAGGGCTCAGAGAGAAGGGTTTTG |
| | AGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAAC |
| | GCCCCCACATCCACAGCAGTGCTGGGGAGCTGGGGATCCCCACCTCAGCCCAGCCT |
| | AGCACCCAGAGAGCAGGAGGCTCCTGGGACCCAATGGCCTCTGGATGAAACGTCAG |
| | GAATTTACTACACAGAAATCAGAGAAAGAGAGAGAGAGAAATCAGAGAAAGGCAG |
| | GCCCCCATGGGCAGCGGTGGTAGGAACGCCCCCACAGGCGCACACCAGCCTACAGC |
| | CCCACCACCACCCATGGGAGCCTTCTGTGAGAGAGAGCCTCTGTTCCACATGGCCCT |
| | GGAAAAATGAGGATTTTAACCAAAAATTCACACAGCTGCTACTTCTACAAAGACCT |
| | CACCCCAGAAGCCAAGATCCCCTGGTCAAGAGAAGCTGGCCTGATTATGTGGAGGA |
| | GAATCGAGGACATTTAATTGAGATCAGAGACTTATTTGGCCCAGGCCTGGATACCC |
| | AAGAACCTCGCATAGTCATACTGCAGGGGGCTGCTGGAATTGGGAAGTCAACACTG |
| | GCCAGGCAGGTGAAGGAAGCCTGGGGGAGAGGCCAGCTGTATGGGGACCGCTTCC |
| | AGCATGTCTTCTACTTCAGCTGCAGAGAGCTGGCCCAGTCCAAGGTGGTGAGTCTCG |
| | CTGAGCTCATCGGAAAAGATGGGACAGCCACTCCGGCTCCCATTAGACAGATCCTG |
| | TCTAGGCCAGAGCGGCTGCTCTTCATCCTCGATGGTGTAGATGAGCCAGGATGGGTC |
| | TTGCAGGAGCCGAGTTCTGAGCTCTGTCTGCACTGGAGCCAGCCACAGCCGGCGGA |
| | TGCACTGCTGGGCAGTTTGCTGGGGAAAACTATACTTCCCGAGGCATCCTTCCTGAT |
| | CACGGCTCGGACCACAGCTCTGCAGAACCTCATTCCTTCTTTGGAGCAGGCACGTTG |
| | GGTAGAGGTCCTGGGGTTCTCTGAGTCCAGCAGGAAGGAATATTTCTACAGATATTT |
| | CACAGATGAAAGGCAAGCAATTAGAGCCTTTAGGTTGGTCAAATCAAACAAAGAGC |
| | TCTGGGCCCTGTGTCTTGTGCCCTGGGTGTCCTGGCTGGCCTGCACTTGCCTGATGCA |
| | GCAGATGAAGCGGAAGGAAAAACTCACACTGACTTCCAAGACCACCACAACCCTCT |
| | GTCTACATTACCTTGCCCAGGCTCTCCAAGCTCAGCCATTGGGACCCCAGCTCAGAG |
| | ACCTCTGCTCTCTGGCTGCTGAGGGCATCTGGCAAAAAAAGACCCTTTTCAGTCCAG |
| | ATGACCTCAGGAAGCATGGGTTAGATGGGGCCATCATCTCCACCTTCTTGAAGATGG |
| | GTATTCTTCAAGAGCACCCCATCCCTCTGAGCTACAGCTTCATTCACCTCTGTTTCCA |
| | AGAGTTCTTTGCAGCAATGTCCTATGTCTTGGAGGATGAGAAGGGGAGAGGTAAAC |
| | ATTCTAATTGCATCATAGATTTGGAAAAGACGCTAGAAGCATATGGAATACATGGC |
| | CTGTTTGGGGCATCAACCACACGTTTCCTATTGGGCCTGTTAAGTGATGAGGGGGAG |
| | AGAGAGATGGAGAACATCTTTCACTGCCGGCTGTCTCAGGGGAGGAACCTGATGCA |
| | GTGGGTCCCGTCCCTGCAGCTGCTGCTGCAGCCACACTCTCTGGAGTCCCTCCACTG |
| | CTTGTACGAGACTCGGAACAAAACGTTCCTGACACAAGTGATGGCCCATTTCGAAG |
| | AAATGGGCATGTGTGTAGAAACAGACATGGAGCTCTTAGTGTGCACTTTCTGCATTA |
| | AATTCAGCCGCCACGTGAAGAAGCTTCAGCTGATTGAGGGCAGGCAGCACAGATCA |
| | ACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTA |
| | AGTGGAAACTCGCTGAGCCACTCTGCAGTGAAGAGTCTTTGTAAGACCCTGAGACG |
| | CCCTCGCTGCCTCCTGGAGACCCTGCGGTTGGCTGGCTGTGGCCTCACAGCTGAGGA |
| | CTGCAAGGACCTTGCCTTTGGGCTGAGAGCCAACCAGACCCTGACCGAGCTGGACC |
| | TGAGCTTCAATGTGCTCACGGATGCTGGAGCCAAACACCTTTGCCAGAGACTGAGA |
| | CAGCCGAGCTGCAAGCTACAGCGACTGCAGCTGGTCAGCTGTGGCCTCACGTCTGA |
| | CTGCTGCCAGGACCTGGCCTCTGTGCTTAGTGCCAGCCCCAGCCTGAAGGAGCTAGA |
| | CCTGCAGCAGAACAACCTGGATGACGTTGGCGTGCGACTGCTCTGTGAGGGGCTCA |
| | GGCATCCTGCCTGCAAACTCATACGCCTGGGGCTGGACCAGACAACTCTGAGTGAT |
| | GAGATGAGGCAGGAACTGAGGGCCCTGGAGCAGGAGAAACCTCAGCTGCTCATCTT |
| | CAGCAGACGGAAACCAAGTGTGATGACCCCTACTGAGGGCCTGGATACGGGAGAG |
| | ATGAGTAATAGCACATCCTCACTCAAGCGGCAGAGACTCGGATCAGAGAGGGCGGC |
| | TTCCCATGTTGCTCAGGCTAATCTCAAACTCCTGGACGTGAGCAAGATCTTCCCAAT |
| | TGCTGAGATTGCAGAGGAAAGCTCCCCAGAGGTAGTACCGGTGGAACTCTTGTGCG |
| | TGCCTTCTCCTGCCTCTCAAGGGGACCTGCATACGAAGCCTTTGGGGACTGACGATG |
| | ACTTCTGGGGCCCCACGGGGCCTGTGGCTACTGAGGTAGTTGACAAAGAAAAGAAC |
| | TTGTACCGAGTTCACTTCCCTGTAGCTGGCTCCTACCGCTGGCCCAACACGGGTCTC |
| | TGCTTTGTGATGAGAGAAGCGGTGACCGTTGAGATTGAATTCTGTGTGTGGGACCAG |
| | TTCCTGGGTGAGATCAACCCACAGCACAGCTGGATGGTGGCAGGGCCTCTGCTGGA |
| | CATCAAGGCTGAGCCTGGAGCTGTGGAAGCTGTGCACCTCCCTCACTTTGTGGCTCT |
| | CCAAGGGGGCCATGTGGACACATCCCTGTTCCAAATGGCCCACTTTAAAGAGGAGG |
| | GGATGCTCCTGGAGAAGCCAGCCAGGGTGGAGCTGCATCACATAGTTCTGGAAAAC |
| | CCCAGCTTCTCCCCCTTGGGAGTCCTCCTGAAAATGATCCATAATGCCCTGCGCTTC |
| | ATTCCCGTCACCTCTGTGGTGTTGCTTTACCACCGCGTCCATCCTGAGGAAGTCACCT |
| | TCCACCTCTACCTGATCCCAAGTGACTGCTCCATTCGGAAGGCCATAGATGATCTAG |
| | AAATGAAATTCCAGTTTGTGCGAATCCACAAGCCACCCCCGCTGACCCCACTTTATA |
| | TGGGCTGTCGTTACACTGTGTCTGGGTCTGGTTCAGGGATGCTGGAAATACTCCCCA |
| | AGGAACTGGAGCTCTGCTATCGAAGCCCTGGAGAAGACCAGCTGTTCTCGGAGTTC |
| | TACGTTGGCCACTTGGGATCAGGGATCAGGCTGCAAGTGAAAGACAAGAAAGATGA |
| | GACTCTGGTGTGGGAGGCCTTGGTGAAACCAGGAGATCTCATGCCTGCAACTACTCT |
| | GATCCCTCCAGCCCGCATAGCCGTACCTTCACCTCTGGATGCCCCGCAGTTGCTGCA |
| | CTTTGTGGACCAGTATCGAGAGCAGCTGATAGCCCGAGTGACATCGGTGGAGGTTG |
| | TCTTGGACAAACTGCATGGACAGGTGCTGAGCCAGGAGCAGTACGAGAGGGTGCTG |
| | GCTGAGAACACGAGGCCCAGCCAGATGCGGAAGCTGTTCAGCTTGAGCCAGTCCTG |
| | GGACCGGAAGTGCAAAGATGGACTCTACCAAGCCCTGAAGGAGACCCATCCTCACC |
| | TCATTATGGAACTCTGGGAGAAGGGCAGCAAAAAGGGACTCCTGCCACTCAGCAGC |
| | TGAAGTATCAACACCAGCCCTTGACCCTTGAGTCCTGGCTTTGGCTGACCCTTCTTTG |
| | GGTCTCAGTTTCTTTCTCTGCAAACAAGTTGCCATCTGGTTTGCCTTCCAGCACTAAA |
| | GTAATGGAACTTTGATGATGCCTTTGCTGGGCATTATGTGTCCATGCCAGGGATGCC |
| | ACAGGGGGCCCCAGTCCAGGTGGCCTAACAGCATCTCAGGGAATGTCCATCTGGAG |
| | CTGGCAAGACCCCTGCAGACCTCATAGAGCCTCATCTGGTGGCCACAGCAGCCAAG |
| | CCTAGAGCCCTCCGGATCCCATCCAGGCGCAAAGAGGAATAGGAGGGACATGGAAC |
| | CATTTGCCTCTGGCTGTGTCACAGGGTGAGCCCCAAAATTGGGGTTCAGCGTGGGAG |
| | GCCACGTGGATTCTTGGCTTTGTACAGGAAGATCTACAAGAGCAAGCCAACAGAGT |
| | AAAGTGGAAGGAAGTTTATTCAGAAAATAAAGGAGTATCACAGCTCTTTTAGAATT |
| | TGTCTAGCAGGCTTTCCAGTTTTTACCAGAAAACCCCTATAAATTAAAAATTTTTTAC |
| | TTAAATTTAAGAATTAAAAAAATACAAAAAAGAAAAAATGAAAATAAAGGAATAA |
| | GAAGTTACCTACTCCA |
| 62 | GTTGACTAGGCGCTGTTCTTGCTGGCTGGTGCCCCAGGGCCTGGAGAGGTCTGAAGA |
| | AACCTGGGAGCCAGCAGCCCGGGGCTCCACTCTGGGTTCTGAAAGCCCATTCCCTGC |
| | TCTGCGGCTCCTCCCACCCCACCTCTTCTCAGCCTTGCAGCTCAAGGGTTGATCTCAG |
| | GAGTCCAGGACCCAGGAGAGGGAAGAATCTGAGGAACACAGAACAGTGAGCGTTG |
| | CCCACACCCCATCTCCCGTCACCACATCTCCCCTCACCCTCACCCTCCCTGCCTGGCC |
| | CTGGACCCCATCCCAGGACCTCCCTATCAGCTGACTTCTTCCAGTGTCTTGCAGGCC |
| | CCTCTGGGCTCCTCCCTCCCCTGGCTTTTCCTACCACTCCCCCTCTATCGGCGTCTAT |
| | CTGTAGGTGCCCTGGGATTTATAAAACTGGGTTCCGAATGCTGAATAAGAGACGGT |
| | AAGAGCCAAGGCAAAGGACAGCACTGTTCTCTGCCTGCCTGATACCCTCACCACCT |
| | GGGAACATCCCCCAGACACCCTCTTAACTCCGGGACAGAGATGGCTGGCGGAGCCT |
| | GGGGCCGCCTGGCCTGTTACTTGGAGTTCCTGAAGAAGGAGGAGCTGAAGGAGTTC |
| | CAGCTTCTGCTCGCCAATAAAGCGCACTCCAGGAGCTCTTCGGGTGAGACACCCGCT |
| | CAGCCAGAGAAGACGAGTGGCATGGAGGTGGCCTCGTACCTGGTGGCTCAGTATGG |
| | GGAGCAGCGGGCCTGGGACCTAGCCCTCCATACCTGGGAGCAGATGGGGCTGAGGT |
| | CACTGTGCGCCCAAGCCCAGGAAGGGGCAGGCCACTCTCCCTCATTCCCCTACAGCC |
| | CAAGTGAACCCCACCTGGGGTCTCCCAGCCAACCCACCTCCACCGCAGTGCTAATGC |
| | CCTGGATCCATGAATTGCCGGCGGGGTGCACCCAGGGCTCAGAGAGAAGGGTTTTG |
| | AGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAAC |
| | GCCCCCACATCCACAGCAGTGCTGGGGAGCTGGGGATCCCCACCTCAGCCCAGCCT |
| | AGCACCCAGAGAGCAGGAGGCTCCTGGGACCCAATGGCCTCTGGATGAAACGTCAG |
| | GAATTTACTACACAGAAATCAGAGAAAGAGAGAGAGAGAAATCAGAGAAAGGCAG |
| | GCCCCCATGGGCAGCGGTGGTAGGAACGCCCCCACAGGCGCACACCAGCCTACAGC |
| | CCCACCACCACCCATGGGAGCCTTCTGTGAGAGAGAGCCTCTGTTCCACATGGCCCT |
| | GGAAAAATGAGGATTTTAACCAAAAATTCACACAGCTGCTACTTCTACAAAGACCT |
| | CACCCCAGAAGCCAAGATCCCCTGGTCAAGAGAAGCTGGCCTGATTATGTGGAGGA |
| | GAATCGAGGACATTTAATTGAGATCAGAGACTTATTTGGCCCAGGCCTGGATACCC |
| | AAGAACCTCGCATAGTCATACTGCAGGGGGCTGCTGGAATTGGGAAGTCAACACTG |
| | GCCAGGCAGGTGAAGGAAGCCTGGGGGAGAGGCCAGCTGTATGGGGACCGCTTCC |
| | AGCATGTCTTCTACTTCAGCTGCAGAGAGCTGGCCCAGTCCAAGGTGGTGAGTCTCG |
| | CTGAGCTCATCGGAAAAGATGGGACAGCCACTCCGGCTCCCATTAGACAGATCCTG |
| | TCTAGGCCAGAGCGGCTGCTCTTCATCCTCGATGGTGTAGATGAGCCAGGATGGGTC |
| | TTGCAGGAGCCGAGTTCTGAGCTCTGTCTGCACTGGAGCCAGCCACAGCCGGCGGA |
| | TGCACTGCTGGGCAGTTTGCTGGGGAAAACTATACTTCCCGAGGCATCCTTCCTGAT |
| | CACGGCTCGGACCACAGCTCTGCAGAACCTCATTCCTTCTTTGGAGCAGGCACGTTG |
| | GGTAGAGGTCCTGGGGTTCTCTGAGTCCAGCAGGAAGGAATATTTCTACAGATATTT |
| | CACAGATGAAAGGCAAGCAATTAGAGCCTTTAGGTTGGTCAAATCAAACAAAGAGC |
| | TCTGGGCCCTGTGTCTTGTGCCCTGGGTGTCCTGGCTGGCCTGCACTTGCCTGATGCA |
| | GCAGATGAAGCGGAAGGAAAAACTCACACTGACTTCCAAGACCACCACAACCCTCT |
| | GTCTACATTACCTTGCCCAGGCTCTCCAAGCTCAGCCATTGGGACCCCAGCTCAGAG |
| | ACCTCTGCTCTCTGGCTGCTGAGGGCATCTGGCAAAAAAAGACCCTTTTCAGTCCAG |
| | ATGACCTCAGGAAGCATGGGTTAGATGGGGCCATCATCTCCACCTTCTTGAAGATGG |
| | GTATTCTTCAAGAGCACCCCATCCCTCTGAGCTACAGCTTCATTCACCTCTGTTTCCA |
| | AGAGTTCTTTGCAGCAATGTCCTATGTCTTGGAGGATGAGAAGGGGAGAGGTAAAC |
| | ATTCTAATTGCATCATAGATTTGGAAAAGACGCTAGAAGCATATGGAATACATGGC |
| | CTGTTTGGGGCATCAACCACACGTTTCCTATTGGGCCTGTTAAGTGATGAGGGGGAG |
| | AGAGAGATGGAGAACATCTTTCACTGCCGGCTGTCTCAGGGGAGGAACCTGATGCA |
| | GTGGGTCCCGTCCCTGCAGCTGCTGCTGCAGCCACACTCTCTGGAGTCCCTCCACTG |
| | CTTGTACGAGACTCGGAACAAAACGTTCCTGACACAAGTGATGGCCCATTTCGAAG |
| | AAATGGGCATGTGTGTAGAAACAGACATGGAGCTCTTAGTGTGCACTTTCTGCATTA |
| | AATTCAGCCGCCACGTGAAGAAGCTTCAGCTGATTGAGGGCAGGCAGCACAGATCA |
| | ACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTA |
| | AGTGGAAACTCGCTGAGCCACTCTGCAGTGAAGAGTCTTTGTAAGACCCTGAGACG |
| | CCCTCGCTGCCTCCTGGAGACCCTGCGGTTGGCTGGCTGTGGCCTCACAGCTGAGGA |
| | CTGCAAGGACCTTGCCTTTGGGCTGAGAGCCAACCAGACCCTGACCGAGCTGGACC |
| | TGAGCTTCAATGTGCTCACGGATGCTGGAGCCAAACACCTTTGCCAGAGACTGAGA |
| | CAGCCGAGCTGCAAGCTACAGCGACTGCAGCTGGTCAGCTGTGGCCTCACGTCTGA |
| | CTGCTGCCAGGACCTGGCCTCTGTGCTTAGTGCCAGCCCCAGCCTGAAGGAGCTAGA |
| | CCTGCAGCAGAACAACCTGGATGACGTTGGCGTGCGACTGCTCTGTGAGGGGCTCA |
| | GGCATCCTGCCTGCAAACTCATACGCCTGGGGCTGGACCAGACAACTCTGAGTGAT |
| | GAGATGAGGCAGGAACTGAGGGCCCTGGAGCAGGAGAAACCTCAGCTGCTCATCTT |
| | CAGCAGACGGAAACCAAGTGTGATGACCCCTACTGAGGGCCTGGATACGGGAGAG |
| | ATGAGTAATAGCACATCCTCACTCAAGCGGCAGAGACTCGGATCAGAGAGGGCGGC |
| | TTCCCATGTTGCTCAGGCTAATCTCAAACTCCTGGACGTGAGCAAGATCTTCCCAAT |
| | TGCTGAGATTGCAGAGGAAAGCTCCCCAGAGGTAGTACCGGTGGAACTCTTGTGCG |
| | TGCCTTCTCCTGCCTCTCAAGGGGACCTGCATACGAAGCCTTTGGGGACTGACGATG |
| | ACTTCTGGGGCCCCACGGGGCCTGTGGCTACTGAGGTAGTTGACAAAGAAAAGAAC |
| | TTGTACCGAGTTCACTTCCCTGTAGCTGGCTCCTACCGCTGGCCCAACACGGGTCTC |
| | TGCTTTGTGATGAGAGAAGCGGTGACCGTTGAGATTGAATTCTGTGTGTGGGACCAG |
| | TTCCTGGGTGAGATCAACCCACAGCACAGCTGGATGGTGGCAGGGCCTCTGCTGGA |
| | CATCAAGGCTGAGCCTGGAGCTGTGGAAGCTGTGCACCTCCCTCACTTTGTGGCTCT |
| | CCAAGGGGGCCATGTGGACACATCCCTGTTCCAAATGGCCCACTTTAAAGAGGAGG |
| | GGATGCTCCTGGAGAAGCCAGCCAGGGTGGAGCTGCATCACATAGTTCTGGAAAAC |
| | CCCAGCTTCTCCCCCTTGGGAGTCCTCCTGAAAATGATCCATAATGCCCTGCGCTTC |
| | ATTCCCGTCACCTCTGTGGTGTTGCTTTACCACCGCGTCCATCCTGAGGAAGTCACCT |
| | TCCACCTCTACCTGATCCCAAGTGACTGCTCCATTCGGAAGGAACTGGAGCTCTGCT |
| | ATCGAAGCCCTGGAGAAGACCAGCTGTTCTCGGAGTTCTACGTTGGCCACTTGGGAT |
| | CAGGGATCAGGCTGCAAGTGAAAGACAAGAAAGATGAGACTCTGGTGTGGGAGGC |
| | CTTGGTGAAACCAGGAGATCTCATGCCTGCAACTACTCTGATCCCTCCAGCCCGCAT |
| | AGCCGTACCTTCACCTCTGGATGCCCCGCAGTTGCTGCACTTTGTGGACCAGTATCG |
| | AGAGCAGCTGATAGCCCGAGTGACATCGGTGGAGGTTGTCTTGGACAAACTGCATG |
| | GACAGGTGCTGAGCCAGGAGCAGTACGAGAGGGTGCTGGCTGAGAACACGAGGCC |
| | CAGCCAGATGCGGAAGCTGTTCAGCTTGAGCCAGTCCTGGGACCGGAAGTGCAAAG |
| | ATGGACTCTACCAAGCCCTGAAGGAGACCCATCCTCACCTCATTATGGAACTCTGGG |
| | AGAAGGGCAGCAAAAAGGGACTCCTGCCACTCAGCAGCTGAAGTATCAACACCAGC |
| | CCTTGACCCTTGAGTCCTGGCTTTGGCTGACCCTTCTTTGGGTCTCAGTTTCTTTCTCT |
| | GCAAACAAGTTGCCATCTGGTTTGCCTTCCAGCACTAAAGTAATGGAACTTTGATGA |
| | TGCCTTTGCTGGGCATTATGTGTCCATGCCAGGGATGCCACAGGGGGCCCCAGTCCA |
| | GGTGGCCTAACAGCATCTCAGGGAATGTCCATCTGGAGCTGGCAAGACCCCTGCAG |
| | ACCTCATAGAGCCTCATCTGGTGGCCACAGCAGCCAAGCCTAGAGCCCTCCGGATC |
| | CCATCCAGGCGCAAAGAGGAATAGGAGGGACATGGAACCATTTGCCTCTGGCTGTG |
| | TCACAGGGTGAGCCCCAAAATTGGGGTTCAGCGTGGGAGGCCACGTGGATTCTTGG |
| | CTTTGTACAGGAAGATCTACAAGAGCAAGCCAACAGAGTAAAGTGGAAGGAAGTTT |
| | ATTCAGAAAATAAAGGAGTATCACAGCTCTTTTAGAATTTGTCTAGCAGGCTTTCCA |
| | GTTTTTACCAGAAAACCCCTATAAATTAAAAATTTTTTACTTAAATTTAAGAATTAA |
| | AAAAATACAAAAAAGAAAAAATGAAAATAAAGGAATAAGAAGTTACCTACTCCA |
| 63 | GCAGCACAGATCAACATGGAGCCCCACCATGGTAGTCCTGTTCAGGTGGGTCCCAG |
| | TCACAGATGCCTATTGGCAGATTCTCTTCTCCGTCCTCAAGGTCACCAGAAACCTGA |
| | AGGAGCTGGACCTAAGTGGAAACTCGCTGAGCCACTCTGCAGTGAAGAGTCTTTGT |
| | AAGACCCTGAGACGCCCTCGCTGCCTCCTGGAGACCCTGCGGTTGGCTGGCTGTGGC |
| | CTCACAGCTGAGGACTGCAAGGACCTTGCCTTTGGGCTGAGAGCCAACCAGACCCT |
| | GACCGAGCTGGACCTGAGCTTCAATGTGCTCACGGATGCTGGAGCCAAACACCTTT |
| | GCCAGAGACTGAGACAGCCGAGCTGCAAGCTACAGCGACTGCAGCTGGTCAGCTGT |
| | GGCCTCACGTCTGACTGCTGCCAGGACCTGGCCTCTGTGCTTAGTGCCAGCCCCAGC |
| | CTGAAGGAGCTAGACCTGCAGCAGAACAACCTGGATGACGTTGGCGTGCGACTGCT |
| | CTGTGAGGGGCTCAGGCATCCTGCCTGCAAACTCATACGCCTGGGGAAACCAAGTG |
| | TGATGACCCCTACTGAGGGCCTGGATACGGGAGAGATGAGTAATAGCACATCCTCA |
| | CTCAAGCGGCAGAGACTCGGATCAGAGAGGGCGGCTTCCCATGTTGCTCAGGCTAA |
| | TCTCAAACTCCTGGACGTGAGCAAGATCTTCCCAATTGCTGAGATTGCAGAGGAAA |
| | GCTCCCCAGAGGTAGTACCGGTGGAACTCTTGTGCGTGCCTTCTCCTGCCTCTCAAG |
| | GGGACCTGCATACGAAGCCTTTGGGGACTGACGATGACTTCTGGGGCCCCACGGGG |
| | CCTGTGGCTACTGAGGTAGTTGACAAAGAAAAGAACTTGTACCGAGTTCACTTCCCT |
| | GTAGCTGGCTCCTACCGCTGGCCCAACACGGGTCTCTGCTTTGTGATGAGAGAAGCG |
| | GTGACCGTTGAGATTGAATTCTGTGTGTGGGACCAGTTCCTGGGTGAGATCAACCCA |
| | CAGCACAGCTGGATGGTGGCAGGGCCTCTGCTGGACATCAAGGCTGAGCCTGGAGC |
| | TGTGGAAGCTGTGCACCTCCCTCACTTTGTGGCTCTCCAAGGGGGCCATGTGGACAC |
| | ATCCCTGTTCCAAATGGCCCACTTTAAAGAGGAGGGGATGCTCCTGGAGAAGCCAG |
| | CCAGGGTGGAGCTGCATCACATAGTTCTGGAAAACCCCAGCTTCTCCCCCTTGGGAG |
| | TCCTCCTGAAAATGATCCATAATGCCCTGCGCTTCATTCCCGTCACCTCTGTGGTGTT |
| | GCTTTACCACCGCGTCCATCCTGAGGAAGTCACCTTCCACCTCTACCTGATCCCAAG |
| | TGACTGCTCCATTCGGAAGGCCATAGATGATCTAGAAATGAAATTCCAGTTTGTGCG |
| | AATCCACAAGCCACCCCCGCTGACCCCACTTTATATGGGCTGTCGTTACACTGTGTC |
| | TGGGTCTGGTTCAGGGATGCTGGAAATACTCCCCAAGGAACTGGAGCTCTGCTATCG |
| | AAGCCCTGGAGAAGACCAGCTGTTCTCGGAGTTCTACGTTGGCCACTTGGGATCAG |
| | GGATCAGGCTGCAAGTGAAAGACAAGAAAGATGAGACTCTGGTGTGGGAGGCCTTG |
| | GTGAAACCAGGAGATCTCATGCCTGCAACTACTCTGATCCCTCCAGCCCGCATAGCC |
| | GTACCTTCACCTCTGGATGCCCCGCAGTTGCTGCACTTTGTGGACCAGTATCGAGAG |
| | CAGCTGATAGCCCGAGTGACATCGGTGGAGGTTGTCTTGGACAAACTGCATGGACA |
| | GGTGCTGAGCCAGGAGCAGTACGAGAGGGTGCTGGCTGAGAACACGAGGCCCAGC |
| | CAGATGCGGAAGCTGTTCAGCTTGAGCCAGTCCTGGGACCGGAAGTGCAAAGATGG |
| | ACTCTACCAAGCCCTGAAGGAGACCCATCCTCACCTCATTATGGAACTCTGGGAGA |
| | AGGGCAGCAAAAAGGGACTCCTGCCACTCAGCAGCTGAAGTATCAACACCAGCCCT |
| | TGACCCTTGAGTCCTGGCTTTGGCTGACCCTTCTTTGGGTCTCAGTTTCTTTCTCTGC |
| | AAACAAGTTGCCATCTGGTTTGCCTTCCAGCACTAAAGTAATGGAACTTTGATGATG |
| | CCTTTGCTGGGCATTATGTGTCCATGCCAGGGATGCCACAGGGGGCCCCAGTCCAGG |
| | TGGCCTAACAGCATCTCAGGGAATGTCCATCTGGAGCTGGCAAGACCCCTGCAGAC |
| | CTCATAGAGCCTCATCTGGTGGCCACAGCAGCCAAGCCTAGAGCCCTCCGGATCCC |
| | ATCCAGGCGCAAAGAGGAATAGGAGGGACATGGAACCATTTGCCTCTGGCTGTGTC |
| | ACAGGGTGAGCCCCAAAATTGGGGTTCAGCGTGGGAGGCCACGTGGATTCTTGGCT |
| | TTGTACAGGAAGATCTACAAGAGCAAGCCAACAGAGTAAAGTGGAAGGAAGTTTAT |
| | TCAGAAAATAAAGGAGTATCACAGCTCTTTTAGAATTTGTCTAGCAGGCTTTCCAGT |
| | TTTTACCAGAAAACCCCTATAAATTAAAAATTTTTTACTTAAATTTAAGAATTAAAA |
| | AAATACAAAAAAGAAAAAATGAAAATAAAGGAATAAGAAGTTACCTACTCCA |
| 64 | GTTGACTAGGCGCTGTTCTTGCTGGCTGGTGCCCCAGGGCCTGGAGAGGTCTGAAGA |
| | AACCTGGGAGCCAGCAGCCCGGGGCTCCACTCTGGGTTCTGAAAGCCCATTCCCTGC |
| | TCTGCGGCTCCTCCCACCCCACCTCTTCTCAGCCTTGCAGCTCAAGGGTTGATCTCAG |
| | GAGTCCAGGACCCAGGAGAGGGAAGAATCTGAGGAACACAGAACAGTGAGCGTTG |
| | CCCACACCCCATCTCCCGTCACCACATCTCCCCTCACCCTCACCCTCCCTGCCTGGCC |
| | CTGGACCCCATCCCAGGACCTCCCTATCAGCTGACTTCTTCCAGTGTCTTGCAGGCC |
| | CCTCTGGGCTCCTCCCTCCCCTGGCTTTTCCTACCACTCCCCCTCTATCGGCGTCTAT |
| | CTGTAGGTGCCCTGGGATTTATAAAACTGGGTTCCGAATGCTGAATAAGAGACGGT |
| | AAGAGCCAAGGCAAAGGACAGCACTGTTCTCTGCCTGCCTGATACCCTCACCACCT |
| | GGGAACATCCCCCAGACACCCTCTTAACTCCGGGACAGAGATGGCTGGCGGAGCCT |
| | GGGGCCGCCTGGCCTGTTACTTGGAGTTCCTGAAGAAGGAGGAGCTGAAGGAGTTC |
| | CAGCTTCTGCTCGCCAATAAAGCGCACTCCAGGAGCTCTTCGGGTGAGACACCCGCT |
| | CAGCCAGAGAAGACGAGTGGCATGGAGGTGGCCTCGTACCTGGTGGCTCAGTATGG |
| | GGAGCAGCGGGCCTGGGACCTAGCCCTCCATACCTGGGAGCAGATGGGGCTGAGGT |
| | CACTGTGCGCCCAAGCCCAGGAAGGGGCAGGCCACTCTCCCTCATTCCCCTACAGCC |
| | CAAGTGAACCCCACCTGGGGTCTCCCAGCCAACCCACCTCCACCGCAGTGCTAATGC |
| | CCTGGATCCATGAATTGCCGGCGGGGTGCACCCAGGGCTCAGAGAGAAGGGTTTTG |
| | AGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAAC |
| | GCCCCCACATCCACAGCAGTGCTGGGGAGCTGGGGATCCCCACCTCAGCCCAGCCT |
| | AGCACCCAGAGAGCAGGAGGCTCCTGGGACCCAATGGCCTCTGGATGAAACGTCAG |
| | GAATTTACTACACAGAAATCAGAGAAAGAGAGAGAGAGAAATCAGAGAAAGGCAG |
| | GCCCCCATGGGCAGCGGTGGTAGGAACGCCCCCACAGGCGCACACCAGCCTACAGC |
| | CCCACCACCACCCATGGGAGCCTTCTGTGAGAGAGAGCCTCTGTTCCACATGGCCCT |
| | GGAAAAATGAGGATTTTAACCAAAAATTCACACAGCTGCTACTTCTACAAAGACCT |
| | CACCCCAGAAGCCAAGATCCCCTGGTCAAGAGAAGCTGGCCTGATTATGTGGAGGA |
| | GAATCGAGGACATTTAATTGAGATCAGAGACTTATTTGGCCCAGGCCTGGATACCC |
| | AAGAACCTCGCATAGTCATACTGCAGGGGGCTGCTGGAATTGGGAAGTCAACACTG |
| | GCCAGGCAGGTGAAGGAAGCCTGGGGGAGAGGCCAGCTGTATGGGGACCGCTTCC |
| | AGCATGTCTTCTACTTCAGCTGCAGAGAGCTGGCCCAGTCCAAGGTGGTGAGTCTCG |
| | CTGAGCTCATCGGAAAAGATGGGACAGCCACTCCGGCTCCCATTAGACAGATCCTG |
| | TCTAGGCCAGAGCGGCTGCTCTTCATCCTCGATGGTGTAGATGAGCCAGGATGGGTC |
| | TTGCAGGAGCCGAGTTCTGAGCTCTGTCTGCACTGGAGCCAGCCACAGCCGGCGGA |
| | TGCACTGCTGGGCAGTTTGCTGGGGAAAACTATACTTCCCGAGGCATCCTTCCTGAT |
| | CACGGCTCGGACCACAGCTCTGCAGAACCTCATTCCTTCTTTGGAGCAGGCACGTTG |
| | GGTAGAGGTCCTGGGGTTCTCTGAGTCCAGCAGGAAGGAATATTTCTACAGATATTT |
| | CACAGATGAAAGGCAAGCAATTAGAGCCTTTAGGTTGGTCAAATCAAACAAAGAGC |
| | TCTGGGCCCTGTGTCTTGTGCCCTGGGTGTCCTGGCTGGCCTGCACTTGCCTGATGCA |
| | GCAGATGAAGCGGAAGGAAAAACTCACACTGACTTCCAAGACCACCACAACCCTCT |
| | GTCTACATTACCTTGCCCAGGCTCTCCAAGCTCAGCCATTGGGACCCCAGCTCAGAG |
| | ACCTCTGCTCTCTGGCTGCTGAGGGCATCTGGCAAAAAAAGACCCTTTTCAGTCCAG |
| | ATGACCTCAGGAAGCATGGGTTAGATGGGGCCATCATCTCCACCTTCTTGAAGATGG |
| | GTATTCTTCAAGAGCACCCCATCCCTCTGAGCTACAGCTTCATTCACCTCTGTTTCCA |
| | AGAGTTCTTTGCAGCAATGTCCTATGTCTTGGAGGATGAGAAGGGGAGAGGTAAAC |
| | ATTCTAATTGCATCATAGATTTGGAAAAGACGCTAGAAGCATATGGAATACATGGC |
| | CTGTTTGGGGCATCAACCACACGTTTCCTATTGGGCCTGTTAAGTGATGAGGGGGAG |
| | AGAGAGATGGAGAACATCTTTCACTGCCGGCTGTCTCAGGGGAGGAACCTGATGCA |
| | GTGGGTCCCGTCCCTGCAGCTGCTGCTGCAGCCACACTCTCTGGAGTCCCTCCACTG |
| | CTTGTACGAGACTCGGAACAAAACGTTCCTGACACAAGTGATGGCCCATTTCGAAG |
| | AAATGGGCATGTGTGTAGAAACAGACATGGAGCTCTTAGTGTGCACTTTCTGCATTA |
| | AATTCAGCCGCCACGTGAAGAAGCTTCAGCTGATTGAGGGCAGGCAGCACAGATCA |
| | ACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTA |
| | AGTGGAAACTCGCTGAGCCACTCTGCAGTGAAGAGTCTTTGTAAGACCCTGAGACG |
| | CCCTCGCTGCCTCCTGGAGACCCTGCGGTTGGCTGGCTGTGGCCTCACAGCTGAGGA |
| | CTGCAAGGACCTTGCCTTTGGGCTGAGAGCCAACCAGACCCTGACCGAGCTGGACC |
| | TGAGCTTCAATGTGCTCACGGATGCTGGAGCCAAACACCTTTGCCAGAGACTGAGA |
| | CAGCCGAGCTGCAAGCTACAGCGACTGCAGCTGGTCAGCTGTGGCCTCACGTCTGA |
| | CTGCTGCCAGGACCTGGCCTCTGTGCTTAGTGCCAGCCCCAGCCTGAAGGAGCTAGA |
| | CCTGCAGCAGAACAACCTGGATGACGTTGGCGTGCGACTGCTCTGTGAGGGGCTCA |
| | GGCATCCTGCCTGCAAACTCATACGCCTGGGGAAACCAAGTGTGATGACCCCTACT |
| | GAGGGCCTGGATACGGGAGAGATGAGTAATAGCACATCCTCACTCAAGCGGCAGAG |
| | ACTCGGATCAGAGAGGGCGGCTTCCCATGTTGCTCAGGCTAATCTCAAACTCCTGGA |
| | CGTGAGCAAGATCTTCCCAATTGCTGAGATTGCAGAGGAAAGCTCCCCAGAGGTAG |
| | TACCGGTGGAACTCTTGTGCGTGCCTTCTCCTGCCTCTCAAGGGGACCTGCATACGA |
| | AGCCTTTGGGGACTGACGATGACTTCTGGGGCCCCACGGGGCCTGTGGCTACTGAG |
| | GTAGTTGACAAAGAAAAGAACTTGTACCGAGTTCACTTCCCTGTAGCTGGCTCCTAC |
| | CGCTGGCCCAACACGGGTCTCTGCTTTGTGATGAGAGAAGCGGTGACCGTTGAGATT |
| | GAATTCTGTGTGTGGGACCAGTTCCTGGGTGAGATCAACCCACAGCACAGCTGGAT |
| | GGTGGCAGGGCCTCTGCTGGACATCAAGGCTGAGCCTGGAGCTGTGGAAGCTGTGC |
| | ACCTCCCTCACTTTGTGGCTCTCCAAGGGGGCCATGTGGACACATCCCTGTTCCAAA |
| | TGGCCCACTTTAAAGAGGAGGGGATGCTCCTGGAGAAGCCAGCCAGGGTGGAGCTG |
| | CATCACATAGTTCTGGAAAACCCCAGCTTCTCCCCCTTGGGAGTCCTCCTGAAAATG |
| | ATCCATAATGCCCTGCGCTTCATTCCCGTCACCTCTGTGGTGTTGCTTTACCACCGCG |
| | TCCATCCTGAGGAAGTCACCTTCCACCTCTACCTGATCCCAAGTGACTGCTCCATTC |
| | GGAAGGAACTGGAGCTCTGCTATCGAAGCCCTGGAGAAGACCAGCTGTTCTCGGAG |
| | TTCTACGTTGGCCACTTGGGATCAGGGATCAGGCTGCAAGTGAAAGACAAGAAAGA |
| | TGAGACTCTGGTGTGGGAGGCCTTGGTGAAACCAGGAGATCTCATGCCTGCAACTA |
| | CTCTGATCCCTCCAGCCCGCATAGCCGTACCTTCACCTCTGGATGCCCCGCAGTTGC |
| | TGCACTTTGTGGACCAGTATCGAGAGCAGCTGATAGCCCGAGTGACATCGGTGGAG |
| | GTTGTCTTGGACAAACTGCATGGACAGGTGCTGAGCCAGGAGCAGTACGAGAGGGT |
| | GCTGGCTGAGAACACGAGGCCCAGCCAGATGCGGAAGCTGTTCAGCTTGAGCCAGT |
| | CCTGGGACCGGAAGTGCAAAGATGGACTCTACCAAGCCCTGAAGGAGACCCATCCT |
| | CACCTCATTATGGAACTCTGGGAGAAGGGCAGCAAAAAGGGACTCCTGCCACTCAG |
| | CAGCTGAAGTATCAACACCAGCCCTTGACCCTTGAGTCCTGGCTTTGGCTGACCCTT |
| | CTTTGGGTCTCAGTTTCTTTCTCTGCAAACAAGTTGCCATCTGGTTTGCCTTCCAGCA |
| | CTAAAGTAATGGAACTTTGATGATGCCTTTGCTGGGCATTATGTGTCCATGCCAGGG |
| | ATGCCACAGGGGGCCCCAGTCCAGGTGGCCTAACAGCATCTCAGGGAATGTCCATC |
| | TGGAGCTGGCAAGACCCCTGCAGACCTCATAGAGCCTCATCTGGTGGCCACAGCAG |
| | CCAAGCCTAGAGCCCTCCGGATCCCATCCAGGCGCAAAGAGGAATAGGAGGGACAT |
| | GGAACCATTTGCCTCTGGCTGTGTCACAGGGTGAGCCCCAAAATTGGGGTTCAGCGT |
| | GGGAGGCCACGTGGATTCTTGGCTTTGTACAGGAAGATCTACAAGAGCAAGCCAAC |
| | AGAGTAAAGTGGAAGGAAGTTTATTCAGAAAATAAAGGAGTATCACAGCTCTTTTA |
| | GAATTTGTCTAGCAGGCTTTCCAGTTTTTACCAGAAAACCCCTATAAATTAAAAATT |
| | TTTTACTTAAATTTAAGAATTAAAAAAATACAAAAAAGAAAAAATGAAAATAAAGG |
| | AATAAGAAGTTACCTACTCCA |
| 65 | GTTGACTAGGCGCTGTTCTTGCTGGCTGGTGCCCCAGGGCCTGGAGAGGTCTGAAGA |
| | AACCTGGGAGCCAGCAGCCCGGGGCTCCACTCTGGGTTCTGAAAGCCCATTCCCTGC |
| | TCTGCGGCTCCTCCCACCCCACCTCTTCTCAGCCTTGCAGCTCAAGGGTTGATCTCAG |
| | GAGTCCAGGACCCAGGAGAGGGAAGAATCTGAGGAACACAGAACAGTGAGCGTTG |
| | CCCACACCCCATCTCCCGTCACCACATCTCCCCTCACCCTCACCCTCCCTGCCTGGCC |
| | CTGGACCCCATCCCAGGACCTCCCTATCAGCTGACTTCTTCCAGTGTCTTGCAGGCC |
| | CCTCTGGGCTCCTCCCTCCCCTGGCTTTTCCTACCACTCCCCCTCTATCGGCGTCTAT |
| | CTGTAGGTGCCCTGGGATTTATAAAACTGGGTTCCGAATGCTGAATAAGAGACGGT |
| | AAGAGCCAAGGCAAAGGACAGCACTGTTCTCTGCCTGCCTGATACCCTCACCACCT |
| | GGGAACATCCCCCAGACACCCTCTTAACTCCGGGACAGAGATGGCTGGCGGAGCCT |
| | GGGGCCGCCTGGCCTGTTACTTGGAGTTCCTGAAGAAGGAGGAGCTGAAGGAGTTC |
| | CAGCTTCTGCTCGCCAATAAAGCGCACTCCAGGAGCTCTTCGGGTGAGACACCCGCT |
| | CAGCCAGAGAAGACGAGTGGCATGGAGGTGGCCTCGTACCTGGTGGCTCAGTATGG |
| | GGAGCAGCGGGCCTGGGACCTAGCCCTCCATACCTGGGAGCAGATGGGGCTGAGGT |
| | CACTGTGCGCCCAAGCCCAGGAAGGGGCAGGCCACTCTCCCTCATTCCCCTACAGCC |
| | CAAGTGAACCCCACCTGGGGTCTCCCAGCCAACCCACCTCCACCGCAGTGCTAATGC |
| | CCTGGATCCATGAATTGCCGGCGGGGTGCACCCAGGGCTCAGAGAGAAGGGTTTTG |
| | AGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAAC |
| | GCCCCCACATCCACAGCAGTGCTGGGGAGCTGGGGATCCCCACCTCAGCCCAGCCT |
| | AGCACCCAGAGAGCAGGAGGCTCCTGGGACCCAATGGCCTCTGGATGAAACGTCAG |
| | GAATTTACTACACAGAAATCAGAGAAAGAGAGAGAGAGAAATCAGAGAAAGGCAG |
| | GCCCCCATGGGCAGCGGTGGTAGGAACGCCCCCACAGGCGCACACCAGCCTACAGC |
| | CCCACCACCACCCATGGGAGCCTTCTGTGAGAGAGAGCCTCTGTTCCACATGGCCCT |
| | GGAAAAATGAGGATTTTAACCAAAAATTCACACAGCTGCTACTTCTACAAAGACCT |
| | CACCCCAGAAGCCAAGATCCCCTGGTCAAGAGAAGCTGGCCTGATTATGTGGAGGA |
| | GAATCGAGGACATTTAATTGAGATCAGAGACTTATTTGGCCCAGGCCTGGATACCC |
| | AAGAACCTCGCATAGTCATACTGCAGGGGGCTGCTGGAATTGGGAAGTCAACACTG |
| | GCCAGGCAGGTGAAGGAAGCCTGGGGGAGAGGCCAGCTGTATGGGGACCGCTTCC |
| | AGCATGTCTTCTACTTCAGCTGCAGAGAGCTGGCCCAGTCCAAGGTGGTGAGTCTCG |
| | CTGAGCTCATCGGAAAAGATGGGACAGCCACTCCGGCTCCCATTAGACAGATCCTG |
| | TCTAGGCCAGAGCGGCTGCTCTTCATCCTCGATGGTGTAGATGAGCCAGGATGGGTC |
| | TTGCAGGAGCCGAGTTCTGAGCTCTGTCTGCACTGGAGCCAGCCACAGCCGGCGGA |
| | TGCACTGCTGGGCAGTTTGCTGGGGAAAACTATACTTCCCGAGGCATCCTTCCTGAT |
| | CACGGCTCGGACCACAGCTCTGCAGAACCTCATTCCTTCTTTGGAGCAGGCACGTTG |
| | GGTAGAGGTCCTGGGGTTCTCTGAGTCCAGCAGGAAGGAATATTTCTACAGATATTT |
| | CACAGATGAAAGGCAAGCAATTAGAGCCTTTAGGTTGGTCAAATCAAACAAAGAGC |
| | TCTGGGCCCTGTGTCTTGTGCCCTGGGTGTCCTGGCTGGCCTGCACTTGCCTGATGCA |
| | GCAGATGAAGCGGAAGGAAAAACTCACACTGACTTCCAAGACCACCACAACCCTCT |
| | GTCTACATTACCTTGCCCAGGCTCTCCAAGCTCAGCCATTGGGACCCCAGCTCAGAG |
| | ACCTCTGCTCTCTGGCTGCTGAGGGCATCTGGCAAAAAAAGACCCTTTTCAGTCCAG |
| | ATGACCTCAGGAAGCATGGGTTAGATGGGGCCATCATCTCCACCTTCTTGAAGATGG |
| | GTATTCTTCAAGAGCACCCCATCCCTCTGAGCTACAGCTTCATTCACCTCTGTTTCCA |
| | AGAGTTCTTTGCAGCAATGTCCTATGTCTTGGAGGATGAGAAGGGGAGAGGTAAAC |
| | ATTCTAATTGCATCATAGATTTGGAAAAGACGCTAGAAGCATATGGAATACATGGC |
| | CTGTTTGGGGCATCAACCACACGTTTCCTATTGGGCCTGTTAAGTGATGAGGGGGAG |
| | AGAGAGATGGAGAACATCTTTCACTGCCGGCTGTCTCAGGGGAGGAACCTGATGCA |
| | GTGGGTCCCGTCCCTGCAGCTGCTGCTGCAGCCACACTCTCTGGAGTCCCTCCACTG |
| | CTTGTACGAGACTCGGAACAAAACGTTCCTGACACAAGTGATGGCCCATTTCGAAG |
| | AAATGGGCATGTGTGTAGAAACAGACATGGAGCTCTTAGTGTGCACTTTCTGCATTA |
| | AATTCAGCCGCCACGTGAAGAAGCTTCAGCTGATTGAGGGCAGGCAGCACAGATCA |
| | ACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTA |
| | AGTGGAAACTCGCTGAGCCACTCTGCAGTGAAGAGTCTTTGTAAGACCCTGAGACG |
| | CCCTCGCTGCCTCCTGGAGACCCTGCGGTTGGCTGGCTGTGGCCTCACAGCTGAGGA |
| | CTGCAAGGACCTTGCCTTTGGGCTGAGAGCCAACCAGACCCTGACCGAGCTGGACC |
| | TGAGCTTCAATGTGCTCACGGATGCTGGAGCCAAACACCTTTGCCAGAGACTGAGA |
| | CAGCCGAGCTGCAAGCTACAGCGACTGCAGCTGGTCAGCTGTGGCCTCACGTCTGA |
| | CTGCTGCCAGGACCTGGCCTCTGTGCTTAGTGCCAGCCCCAGCCTGAAGGAGCTAGA |
| | CCTGCAGCAGAACAACCTGGATGACGTTGGCGTGCGACTGCTCTGTGAGGGGCTCA |
| | GGCATCCTGCCTGCAAACTCATACGCCTGGGGCTGGACCAGACAACTCTGAGTGAT |
| | GAGATGAGGCAGGAACTGAGGGCCCTGGAGCAGGAGAAACCTCAGCTGCTCATCTT |
| | CAGCAGACGGAAACCAAGTGTGATGACCCCTACTGAGGGCCTGGATACGGGAGAG |
| | ATGAGTAATAGCACATCCTCACTCAAGCGGCAGAGACTCGGATCAGAGAGGGCGGC |
| | TTCCCATGTTGCTCAGGCTAATCTCAAACTCCTGGACGTGAGCAAGATCTTCCCAAT |
| | TGCTGAGATTGCAGGCAAGAGCCACGAGGAAAGCTCCCCAGAGGTAGTACCGGTGG |
| | AACTCTTGTGCGTGCCTTCTCCTGCCTCTCAAGGGGACCTGCATACGAAGCCTTTGG |
| | GGACTGACGATGACTTCTGGGGCCCCACGGGGCCTGTGGCTACTGAGGTAGTTGAC |
| | AAAGAAAAGAACTTGTACCGAGTTCACTTCCCTGTAGCTGGCTCCTACCGCTGGCCC |
| | AACACGGGTCTCTGCTTTGTGATGAGAGAAGCGGTGACCGTTGAGATTGAATTCTGT |
| | GTGTGGGACCAGTTCCTGGGTGAGATCAACCCACAGCACAGCTGGATGGTGGCAGG |
| | GCCTCTGCTGGACATCAAGGCTGAGCCTGGAGCTGTGGAAGCTGTGCACCTCCCTCA |
| | CTTTGTGGCTCTCCAAGGGGGCCATGTGGACACATCCCTGTTCCAAATGGCCCACTT |
| | TAAAGAGGAGGGGATGCTCCTGGAGAAGCCAGCCAGGGTGGAGCTGCATCACATA |
| | GTTCTGGAAAACCCCAGCTTCTCCCCCTTGGGAGTCCTCCTGAAAATGATCCATAAT |
| | GCCCTGCGCTTCATTCCCGTCACCTCTGTGGTGTTGCTTTACCACCGCGTCCATCCTG |
| | AGGAAGTCACCTTCCACCTCTACCTGATCCCAAGTGACTGCTCCATTCGGAAGGCCA |
| | TAGATGATCTAGAAATGAAATTCCAGTTTGTGCGAATCCACAAGCCACCCCCGCTGA |
| | CCCCACTTTATATGGGCTGTCGTTACACTGTGTCTGGGTCTGGTTCAGGGATGCTGG |
| | AAATACTCCCCAAGGAACTGGAGCTCTGCTATCGAAGCCCTGGAGAAGACCAGCTG |
| | TTCTCGGAGTTCTACGTTGGCCACTTGGGATCAGGGATCAGGCTGCAAGTGAAAGA |
| | CAAGAAAGATGAGACTCTGGTGTGGGAGGCCTTGGTGAAACCAGGAAGGAACACC |
| | AGCCAGCCGTGGAACCTCAGGTGCAACAGAGACGCCAGGAGATACTAGTGCCCAGC |
| | AGCCTGCGGCAGTACCAATGAAGCCAGAGAGGGCTTGGTGGATGACAAGGAGGCCT |
| | GAGTAGACCGCAGGTGGGTCTGAGAAATGGGCTTAGGTGAGGCAGGTCTTTGAAGG |
| | ATTTGTTCTTAATCATATGCGAGATGCTCAAAAGGCTGGATGCCTGCTTTTGTGGGT |
| | GAAGAGCAAGAAGAGAAAACAGGTTGTACACATACAGATGCAGATGGAGAGACAG |
| | AGAAAAAAAAGGAAGAAGGCAGAGAAATGCACCAATTCTTGAGCTGTATTATCTCT |
| | GGACCTTGGGATTGTGGGAGGCTTTATTTTACTACTGATTTTGCCTACACTGTTTTCT |
| | CAATTTCTAGTTTTCTACAAAGATGATGTGTTAGCTTTTTCACGCATTAAGATTAAAA |
| | TTTAAAACAGACCA |

1. A compound comprising an antisense oligonucleotide of 10 to 30 nucleotides in length and comprising at least 8 contiguous nucleotides of any one of SEQ ID NOs:1 to SEQ ID NO:52, and which inhibits the expression of NLRP3 and/or NLRP1 mRNA.

2. The compound of claim 1, wherein the antisense oligonucleotide is complementary to an equal length segment of human NLRP3 and/or NLRP1 mRNA, with the proviso that the antisense oligonucleotide may contain from one to four inosine bases.

3. The compound of claim 1 or 2, wherein the oligonucleotide is at least 12 nucleotides in length.

4. The compound of claim 3, wherein the oligonucleotide is at least 14 nucleotides in length.

5. The compound of claim 2, wherein the oligonucleotide is from 10 to 24 nucleotides in length.

6. The compound of claim 5, wherein the oligonucleotide is 12, 13, 14, 15, 16, 17, 18, 19, or 20 nucleotides in length.

7. The compound of claim 6, wherein the oligonucleotide is 14 nucleotides in length.

8. The compound of any one of claims 1 to 7, wherein the oligonucleotide comprises at least 10 or at least 12 or at least 14 contiguous nucleobases of any one of SEQ ID NOs: 1 to 52.

9. The compound of claim 8, wherein the oligonucleotide has a nucleobase sequence of any one of SEQ ID NOS: 1 to 52.

10. The compound of claim 8 or 9, wherein the oligonucleotide recruits an endogenous nuclease when hybridized with RNA.

11. The compound of claim 10, wherein the antisense oligonucleotide has a stretch of at least 6 DNA nucleotides sufficient to recruit RNaseH, and optionally a stretch of at least 8 DNA nucleotides.

12. The compound of claim 11, wherein one or more DNA nucleotides comprise a 2′ chemical modification independently selected from 2′-Fluoro, 2′-Methyl, and 2′-Ethyl.

13. The compound of claim 11 or 12, wherein the antisense oligonucleotide is a gapmer having a 5′ and a 3′ segment, each of the 5′ and 3′ segments being from 2 to 6 nucleotides or from 2 to 4 nucleotides, and where the 5′ and 3′ segments do not contain DNA nucleotides.

14. The compound of claim 13, wherein the 5′ and 3′ segments are each 3 nucleotides in length, and the 5′ and 3′ segments flank an internal sequence of 8 DNA nucleotides.

15. The compound of claim 14, wherein one or more nucleotides of the 5′ segment and the 3′ segment comprise 2′-O substituents, optionally where all of the nucleotides of the 5′ segment and the 3′ segment comprise 2′-O substituents.

16. The compound of claim 15, wherein the 2′-O substituents are independently selected from 2′-0 methyl, 2′-0 ethyl, 2′-0 methoxyethyl (MOE), and a bridged nucleotide having a 2′ to 4′ bridge.

17. The compound of claim 16, wherein the bridged nucleotide has a methylene bridge (LNA) or a constrained ethyl bridge (cEt).

18. The compound of any one of claims 1 to 17, wherein at least one nucleotide is unlocked nucleic acid (UNA).

19. The compound of any one of claims 1 to 10, wherein the compound is a FANA antisense oligonucleotide.

20. The compound of any one of claims 1 to 19, wherein the antisense oligonucleotide has a modified backbone.

21. The compound of claim 20, wherein the antisense oligonucleotide a comprises one or more phosphorothioate or phosphorodithioate nucleotides.

22. The compound of claim 21, wherein the oligonucleotide is fully phosphorothioate or phosphorodithioate linked.

23. The compound of any one of claims 1 to 9, wherein the antisense oligonucleotide has a morpholino or thiomorpholino backbone.

24. The compound of any one of claims 1 to 23, wherein cytidine nucleobases in the antisense oligonucleotide are 5-methyl cytidine.

25. The compound of any one of claims 1 to 24, wherein the antisense oligonucleotide has from one to three Inosine nucleobases, and which are optionally deoxyInosine.

26. The compound of claim 1, wherein the oligonucleotide is shown in Table 2 or Table 5.

27. The compound of any one of claims 1 to 26, further comprising a cell targeting or cell penetrating moiety.

28. The compound of claim 27, wherein the cell targeting or cell penetrating moiety is conjugated directly or indirectly to the 3′ end of the oligonucleotide, optionally though a linker.

29. A pharmaceutical composition comprising a compound of any one of claims 1 to 28, further comprising a pharmaceutically acceptable vehicle.

30. The pharmaceutical composition of claim 29, wherein the antisense oligonucleotide is encapsulated in a particle.

31. The pharmaceutical composition of claim 30, wherein the particle is a liposome, polymeric nanoparticle, lipid nanoparticle, or exosome.

32. The pharmaceutical composition of claim 31, wherein the particle is polymeric, and is optionally a di- or tri-block co-polymer.

33. The pharmaceutical composition of claim 31, wherein the particle is a lipid nanoparticle.

34. The pharmaceutical composition of claim 31, wherein the particle is an exosome.

35. The pharmaceutical composition of any one of claims 29 to 34, wherein the composition is formulated for parenteral administration.

36. The pharmaceutical composition of claim 35, wherein the composition is formulated for intravenous, subcutaneous, intradermal, intramuscular, intratumoral, or intrathecal administration.

37. A method for treating a subject having a disease or condition associated with NLRP3 and/or NLRP1 expression or activation of the NLRP3 and/or NLRP1 inflammasome, comprising: administering a compound of any one of claims 1 to 28 or a composition of any one of claims 29 to 36 to the subject.

38. The method of claim 37, wherein the disease or condition is an inflammatory disease, an autoimmune disease, a respiratory disease, or a neurodegenerative disease.

39. The method of claim 37, wherein the disease or condition is a neurodegenerative disease, and which is optionally amyotrophic lateral sclerosis (ALS), Parkinson's disease, Alzheimer's disease, multiple sclerosis, or Huntington's disease.

40. The method of claim 37, wherein the disease or condition comprises a nerve injury.

41. The method of claim 40, wherein the nerve injury comprises a peripheral nerve injury or a spinal cord injury.

42. The method of claim 37, wherein the disease or condition is migraine, traumatic brain injury, myocardial infarction, or stroke.

43. The method of claim 37, wherein the disease or condition is diabetes mellitus or inflammatory bowel syndrome (IBD).

44. The method of claim 37, wherein the disease or condition is neutrophilic asthma.


SEQUENCE LISTING (65 sequences)

SEQ ID NO: 1 - DNA, 14 bp, synthetic construct
  source 1..14: mol_type = other DNA
tggaatatatcgag

SEQ ID NO: 2 - DNA, 14 bp, synthetic construct
  source 1..14: mol_type = other DNA
ggaatatatcgagc

SEQ ID NO: 3 - DNA, 14 bp, synthetic construct
  source 1..14: mol_type = other DNA
gaatatatcgagca

SEQ ID NO: 4 - DNA, 14 bp, synthetic construct
  source 1..14: mol_type = other DNA
aactattaagcaac

SEQ ID NO: 5 - DNA, 14 bp, synthetic construct
  source 1..14: mol_type = other DNA
actattaagcaacg

SEQ ID NO: 6 - DNA, 14 bp, synthetic construct
  source 1..14: mol_type = other DNA
ctattaagcaacgg

SEQ ID NO: 7 - DNA, 14 bp, synthetic construct
  source 1..14: mol_type = other DNA
gtattacagtttac

SEQ ID NO: 8 - DNA, 14 bp, synthetic construct
  source 1..14: mol_type = other DNA
tattacagtttacg

SEQ ID NO: 9 - DNA, 14 bp, synthetic construct
  source 1..14: mol_type = other DNA
attacagtttacgg

SEQ ID NO: 10 - DNA, 14 bp, synthetic construct
  source 1..14: mol_type = other DNA
ccttttcgaatttg

SEQ ID NO: 11 - DNA, 14 bp, synthetic construct
  source 1..14: mol_type = other DNA
cttttcgaatttgc

SEQ ID NO: 12 - DNA, 14 bp, synthetic construct
  source 1..14: mol_type = other DNA
ttttcgaatttgcc

SEQ ID NO: 13 - DNA, 14 bp, synthetic construct
  source 1..14: mol_type = other DNA
cttaatttcttctc

SEQ ID NO: 14 - DNA, 14 bp, synthetic construct
  source 1..14: mol_type = other DNA
acttaatttcttct

SEQ ID NO: 15 - DNA, 14 bp, synthetic construct
  source 1..14: mol_type = other DNA
aacttaatttcttc

SEQ ID NO: 16 - DNA, 14 bp, synthetic construct
  source 1..14: mol_type = other DNA
caacttaatttctt

SEQ ID NO: 17 - DNA, 14 bp, synthetic construct
  source 1..14: mol_type = other DNA
gcaacttaatttct

SEQ ID NO: 18 - DNA, 14 bp, synthetic construct
  source 1..14: mol_type = other DNA
tgcaacttaatttc

SEQ ID NO: 19 - DNA, 14 bp, synthetic construct
  source 1..14: mol_type = other DNA
ttgcaacttaattt

SEQ ID NO: 20 - DNA, 14 bp, synthetic construct
  source 1..14: mol_type = other DNA
cttgcaacttaatt

SEQ ID NO: 21 - DNA, 14 bp, synthetic construct
  source 1..14: mol_type = other DNA
tcttgcaacttaat

SEQ ID NO: 22 - DNA, 14 bp, synthetic construct
  source 1..14: mol_type = other DNA
atcttgcaacttaa

SEQ ID NO: 23 - DNA, 14 bp, synthetic construct
  source 1..14: mol_type = other DNA
agatcttgcaactt

SEQ ID NO: 24 - DNA, 14 bp, synthetic construct
  source 1..14: mol_type = other DNA
agagatcttgcaac

SEQ ID NO: 25 - DNA, 14 bp, synthetic construct
  source 1..14: mol_type = other DNA
tgagagatcttgca

SEQ ID NO: 26 - DNA, 14 bp, synthetic construct
  source 1..14: mol_type = other DNA
gctgagagatcttg

SEQ ID NO: 27 - DNA, 14 bp, synthetic construct
  source 1..14: mol_type = other DNA
ttgctgagagatct

SEQ ID NO: 28 - DNA, 14 bp, synthetic construct
  source 1..14: mol_type = other DNA
tttgctgagagatc

SEQ ID NO: 29 - DNA, 14 bp, synthetic construct
  source 1..14: mol_type = other DNA
atttgctgagagat

SEQ ID NO: 30 - DNA, 14 bp, synthetic construct
  source 1..14: mol_type = other DNA
gatttgctgagaga

SEQ ID NO: 31 - DNA, 14 bp, synthetic construct
  source 1..14: mol_type = other DNA
tgatttgctgagag

SEQ ID NO: 32 - DNA, 14 bp, synthetic construct
  source 1..14: mol_type = other DNA
ctgatttgctgaga

SEQ ID NO: 33 - DNA, 14 bp, synthetic construct
  source 1..14: mol_type = other DNA
cctgatttgctgag

SEQ ID NO: 34 - DNA, 14 bp, synthetic construct
  source 1..14: mol_type = other DNA
gcctgatttgctga

SEQ ID NO: 35 - DNA, 14 bp, synthetic construct
  source 1..14: mol_type = other DNA
agcctgatttgctg

SEQ ID NO: 36 - DNA, 14 bp, synthetic construct
  source 1..14: mol_type = other DNA
cagcctgatttgct

SEQ ID NO: 37 - DNA, 15 bp, synthetic construct
  source 1..15: mol_type = other DNA
  modified_base 12: mod_base = OTHER; note = deoxyinosine
  modified_base 13: mod_base = OTHER; note = deoxyinosine
aggcaggcagannac

SEQ ID NO: 38 - DNA, 17 bp, synthetic construct
  source 1..17: mol_type = other DNA
  modified_base 3: mod_base = OTHER; note = deoxyinosine
  modified_base 13: mod_base = OTHER; note = deoxyinosine
  modified_base 14: mod_base = OTHER; note = deoxyinosine
ctncctggccagnnttg

SEQ ID NO: 39 - DNA, 14 bp, synthetic construct
  source 1..14: mol_type = other DNA
  modified_base 3: mod_base = OTHER; note = deoxyinosine
gangcctcgggaag

SEQ ID NO: 40 - DNA, 14 bp, synthetic construct
  source 1..14: mol_type = other DNA
  modified_base 6: mod_base = OTHER; note = deoxyinosine
  modified_base 8: mod_base = OTHER; note = deoxyinosine
  modified_base 12: mod_base = OTHER; note = deoxyinosine
tcatcngngaanta

SEQ ID NO: 41 - DNA, 14 bp, synthetic construct
  source 1..14: mol_type = other DNA
gtggtggtcttgga

SEQ ID NO: 42 - DNA, 20 bp, synthetic construct
  source 1..20: mol_type = other DNA
  modified_base 3: mod_base = OTHER; note = deoxyinosine
  modified_base 11: mod_base = OTHER; note = deoxyinosine
tcngcagccanagagcagag

SEQ ID NO: 43 - DNA, 17 bp, synthetic construct
  source 1..17: mol_type = other DNA
  modified_base 8: mod_base = OTHER; note = deoxyinosine
gcagccanagagcagag

SEQ ID NO: 44 - DNA, 17 bp, synthetic construct
  source 1..17: mol_type = other DNA
  modified_base 6: mod_base = OTHER; note = deoxyinosine
ccatgnttcctgaggtc

SEQ ID NO: 45 - DNA, 16 bp, synthetic construct
  source 1..16: mol_type = other DNA
  modified_base 4: mod_base = OTHER; note = deoxyinosine
gtgnatgaagctgtag

SEQ ID NO: 46 - DNA, 19 bp, synthetic construct
  source 1..19: mol_type = other DNA
  modified_base 5: mod_base = OTHER; note = deoxyinosine
  modified_base 8: mod_base = OTHER; note = deoxyinosine
acatngcngcaaagaactc

SEQ ID NO: 47 - DNA, 18 bp, synthetic construct
  source 1..18: mol_type = other DNA
  modified_base 12: mod_base = OTHER; note = deoxyinosine
gcaaagaactcntggaaa

SEQ ID NO: 48 - DNA, 19 bp, synthetic construct
  source 1..19: mol_type = other DNA
  modified_base 7: mod_base = OTHER; note = deoxyinosine
  modified_base 10: mod_base = OTHER; note = deoxyinosine
caggccnaanaggaaacgt

SEQ ID NO: 49 - DNA, 18 bp, synthetic construct
  source 1..18: mol_type = other DNA
  modified_base 11: mod_base = OTHER; note = deoxyinosine
  modified_base 16: mod_base = OTHER; note = deoxyinosine
tctcgtacaancagtnga

SEQ ID NO: 50 - DNA, 14 bp, synthetic construct
  source 1..14: mol_type = other DNA
  modified_base 3: mod_base = OTHER; note = deoxyinosine
  modified_base 6: mod_base = OTHER; note = deoxyinosine
  modified_base 9: mod_base = OTHER; note = deoxyinosine
agnacnganaagag

SEQ ID NO: 51 - DNA, 14 bp, synthetic construct
  source 1..14: mol_type = other DNA
ctcaggtccagctc

SEQ ID NO: 52 - DNA, 14 bp, synthetic construct
  source 1..14: mol_type = other DNA
  modified_base 3: mod_base = OTHER; note = deoxyinosine
  modified_base 9: mod_base = OTHER; note = deoxyinosine
  modified_base 12: mod_base = OTHER; note = deoxyinosine
agntcctgncanca

SEQ ID NO: 53 - DNA, 32306 bp, synthetic construct
  source 1..32306: mol_type = other DNA
gcatttcctctctagctgttcctgaggctggcatctgggtaagtccagctccgcggggcagtgtgggcgacaggaccgggagagcagtgggggagattgtgttcagagggcagaacttgtcctggttaatgcactaggaatttcttctggcgtctccaaggttgtgcttcaacaacatttctggaatgttcttttatcattatttgggctctacgtgtgcaagttcgtgtgtgtgtatgtggcggggggaagcggggaggacagcggcagcaaccatagtttacacccacgacttcagcagatttgcccagtatccctcccaggggagaagatggatgggatatacagaaggaaacaaacctccttaaagaccaaagggcagcagatttttgtgctggtgttagggtggaaggagcacagaagaaaggcagaatcctgatgcagatggaaggactgatggggcaggagctgggagggtatgcggagctaatagggctgtctggagagcagtcacctcaggatgggcaggagctgggagggtatgcggagctaatagggctgtctggagagcagtcacctcaggatgcacagggctgggtttgcagggctcaggaggcgaggagtagataggcaggaatggagatgggacgggagagatgagtgagtctgaggcatcttgacattgaactgcagtcacagaaattccaatttggagagctccaccgttttccaggctctctgcctcccttgagtgttatttatggctttaggttgaggtgctttgccaaatagagtataatcaagggatcaaggagggaacaattccaaacaaagccaacttgttcatccatccggactctaggagatatggaagatctaaagatacagcaggtgattgctagtttttgtggaactcacagtctagttgggaagactatgttggtgaatgctgcagtgatacacctgtgataaatgctggggaagtgtgtcttttagtcatctattttgttgtctttgtctttgtctgactgaaacaagtgatggaagacatggtctcatatctctggtcaaaatcctggctctgttccttcctggttctaaacccctcggcaggcttcctagtccccctaagactcagtttatgcatctataaaatggggctaggaataggtgcacctcatagagctctgtgcaggttagatggggaaaaacatcagtgagtgcctagcctgtggaaagctctaaattaatgtgaattataatgataacaattgcgaattttgcaaggagacattaaaggatgtatgtttttatttattttattttattctattttattttttgagatggagttttgctcttattgcccaggctggagtgcagtggtgtgatcttggctcatcgcaacctccgcctcctgggttcaagcgattctcctgcctcagcctcccgagtagctgggattacaggtgcctggtaccatgcccggttaatttttgtatttttagtagagacgggatttctccctgttgtttggtgagcctggtctccaactcctgacctcaggtgatccgcccacctcggcctcccaaagtgctgggattacagggatgtatgtttttattattctccattatgcactcccagcttcatctcacttcaatccactggttgatagaagtgcagtcaaagactgtcccctcctctgcaagcttttatgttctctctcctctctccctagccacgtcctgttctgttatcaacacacttactcacctcctggccttctccttctattgtctctcatccctcttccctcacaaaaacagaagcaaagagccagagccttcagtttggaggaactgaaaacattctcttctgctttctcattttgtagatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggtgagtggaaggaagacttttaaaaaaaattgtggccaagtgcacatagtgtacaattttccatctttatatatatatatatatatatttttttttgagacggagttgctcttgttgcccaggctggagtgcaatggtgagatcttggctcactgcaacctccacctcccgggttcaagcgattttcctgcctcagcctcccaagtagctgggattacaggcattgcacctccacgcccggctaattttgtatttttagtagagatggggtttctccatgttggtcaggttggtctcaaactcccgacctcaggtgatccacccgcctcggcctcgcaaagtgctggaattacaggtgtgagccaccgcgcccggccgtaataatttttaaatgtacagttcagtagtgttaggtacactcacattgtgcaaccagtctccagaactgttttcatcttgcaaaactgaaactcctcacccattgaatgataactcctcattgcctcattctcccagccctggtaaccaccattctatttgccatctctgtgaatcttactactctgggtacctcatataagtggaaacatacagcattttttcctttatgagtagtttatttcactcagcataatgtcctccgggttcaactatgttgtagcatgtgtcggaattcccttcctttaaaaaagactgtataaatactccatggtaccatataccaaattttgtttagttatctgttgatgaacacctggattacttctacctttttgttattgttaatgctgttatgagtgttggtgcacaaatatctcttcaagaccctgctttgaattctctggggtatatactcagaagtgaaatttctgaatcatacggtaattctgttaaatattttgaagaactgctctactgttttagataccatctgcaccatttcccaccaacagcacacaagggttccaatttctccatatcctaagaacacgttatttttatttattttttcttttttatagtagccattctaatgagtgtgaggtggtatctcactgtagttttgatttccgtttccctaatgataagtgatgctgagcacattttcactgcttgttggccatttatagagcttcttcagagaaagttctgttcaagttcttttctcattttcagaccaggtttggcttttgttgttgccttgtaggagttctttatgtattctagatattaaccccatatcagatatatgatttacaaatattttctcctattctgtgggttgtcttttcatttggttaactgtgtcctttgatgcacagaagttagtatgatttttttttttttaaatcatggccgggcatggtggctcatgcctgtaattccagcactttgtgaggctgaggtgggtggatcacctgaggtcaggagttcaacaccagcctggccaacatggcaaaaccccatctctactaaaaatacaaaaaaaattagccgggtgtggtggtgggcacctgtaatcccagctactcaggaggctgaggtaggagaattgcttgaacatggggagtggaggttgcagtgagctgagattgcaccacggcactccagcctgggctaaagagcaagaccctgtttcaaaaaaaaatttttttttcttgtattgtcttggttttttctcttttcccctctcacggtagtgctatgagaaggtggaggcaaactgtttgctttctctaactcctttaaaaagctgatgcagactgtacctaccccctgggcacctgccatgattagtccctctttatctgaatctcagcagtgcctagacctgcctccaattctccatccagcttctcagaccacatgttttgtccagtatctgctgtgtggtcagcaggcttgggcctctgcttcctatggcccagagggcagcctctctccttgaagaaccgtggagtttggggcagaagcctttcctttcaccaccactttgggaattggaatgtaaaagaaattctcttaatccccatgtacacattggggataaggttctttcttttgtcttgtgttctaggcaaaggtagggctcagagaagggcagggtattgcaggatgatgatgtagagctggtggtgatgggaaggaggagtacaaatgagtcagtggatagggagacacatgttaaggcagggcagcagtgtgggtgtaatgacactggggaaacagacagcagtgtgcagccgggggaggtcagaaaccagttgctgcaatggctctcacctgtagtcccagctactcaggaggctaaggcaggaggatcgcttgagcccaggagctcaaaaccagcctgagcaacatagggagacctcatctctaagtaaataaagcaattatcattgtgtcagtacttatatactcacatacccaaatgaaagtgtcataatcacaactgtagaagtactttgctaagcagtttttatggattttatggcacattcaagaaacccatgaaacagcttctggtattatccctcctgcaagtgaatgaggtgagcctggaagagatcctaactggcccatggtttatttctagccataaccagagctggggtaaaaagcagggcatgtctctgatcaaagcctggctcttaaccactacaccgtctttgcttccatatctggattttgaggcagaaaagttataactagggttagaagtttgtttgtttgtttcctggggtggggttaggttttaggtaatagactttggctaatttactgtcatggctcctacctgagtcccagttactcaggtggctgaagtgggtgcatcacttgagctcaggagttccaggctggagtgagctatgaccatgccactgccctccagctttggaaacaaagcaagactctgtctcttaaaaaaaaagaataagattttggttttggcttagattttttggattttattttgtgatatggttaccaagtagacagctgtcagaggtcttttatgggctgggcgcagtggcttatgcctgtaatcccagcacttcaggaggccaagatgggaggattgcttgagcccaggagtttgagaccagcctgggcaacatagtgagaacctgtctctacaaaaaatgcaaacattagttaggtgtggtggcacgtgtcaatagtcccagctgttctggaggctgaggtgggaggatcacttgagcccaggaggttgaggctgcagggagccatgattgtgtcattgcactccagcctgggcaatagagtgaggccctgtctcaaaaaaaaaaaaaaaatcatttatgttacttgtagattcctgcccacctgaagttatctgtcttatatatttcccctagtgactaactcttgttttacctaacctgaatttctcctatagaatgccttcttaagatatgctatctagtgactgtttggatttgtgattctcaacacagatgcatgttggaatcacctgggagcttttagtatgtgatggttcctgggcctcacctcagagggactgaaaaagaacctctgggcgtgcagtcctgaagtccatatttttaaagcttcctgggtgattctaaagtcagccaggattcccaaccactagattaggcacgaaatagactggtcgttctcactcttgaagaaccagacatctagacttgacaatagaagacgtgtgtaaaatggattgcagggaactgcggtcagggaagggtgacctttcttagcccacagccgtgttggggatataggagagcttggagatgccctcacaatcaggctgccaggggcaggcaaaagcaaacgagggtaaaagaaacttagcctgcttcgcatgtttgctgattgatttccaggcctgaggaagagattctgagactggggacagtgggaacacatgcttggcaggtggacagcagaagttgttttgaaactaggagtgcagaaatgctcccaaccagacttttgactggatacctgtcctctgcaactcagagccatgttttgatttgggggatgtttggggtctcctctctcatgccaaatattaggccaggtttcaattgcatcctctgtgataatagttctgggttttgacaccttttttttcccttttaggttcagataatgcacgtgtttcgaatcccactgtgatatgccaggaagacagcattgaagaggagtggatgggtttactggagtacctttcgagaatctctatttgtaaaatgaagaaaggtaagcgactggggtggtgcttctagttgagttttaagacctggttcaggaggctctgggaagctgagcagctgggtaacttggccatactataggttcctgctctttggaatgcaaaggcctgtctcaggaaatccattgtcagttgaagaaacaaattcatgtttgtgtggctcttgcagtcccagagccagcggcggtgattaagcgtttcaagtttgataaggcataggatgcagattacttaaaggaataagattgattcaaatgatctttgggctggtagctacacatagagatgggccttctgctcagctgtgtatcttaaaaacttgacaccagagattctcggcacctttcctacccagtggggaggagctgcaccttccatttctccattccggttaccactcgcttccgatgacaggccccagctgagagcgcatctcaggtggatgtgtgtatactttccccctaacttcctgtctttgccgtagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggtaaggaaactcggcttccaggtgcttcctcctgcttcctcgccagcttcttcttggcgcttgcctcctctcatctcttttcaactatcttccaaatactgttgccacagctacatcataatgccaccactgtctgtttgagactccttcatgagcaaagattgatgtatggtaggtggataaatgggatgaggaaaaaaaaaataaaacaaggaacaaatgtttggggaatgccagtttagcacaaggtattaagtaggatgtaggattgcctacaaatatttgtcaactgaaatttcttgtaagctacttggagcactagtgcctaagagtcagtcaatgtctgtggggcagaacaatgccagagagtctgtgtcccaggagcgaggggacagagggcctcagagctggaggctggggggatgtttcttagaaatgtgcaatctaagcctcctttaaggaatggtgggacctgggcgcatagagaagaaattaaaggaattctgcactgaagactggtatgagccaaatcatgaatgcagtctgagaaaaagtgtgtgcaggtgacagtggagagagacacagtttccttggaaaaaaacatgatgaaggtaaatcaagggaaatagagcagaaatgtggagaccctctttctaggcccccatgttgctctgcactgggcattgccaaaaccccgttttcgggtctgttcacacacttgctgggtggttggaagctagtggccaagtctgtgcaatgaagtggttgagtcacctgacttctgagtcccttttgggtttatcgttcagtgcctcctgcagaaggaccacccacagtggttgtaggaaggtggccaccctgtgatgtgtgagtttagcctggatagaagacacttgacgtggggcaaaattaagggaaacaggaggcttggcccttccacaggcaacaatgagtttcagctttcttgatccgttgacattaccatgaccaggaccaccttcctagtcatggtttggccatgaaggactgactgcccacagggccgcagtggtggtgaagacccaggtcctggtcacagggacttgaggtctggctgtgggacagtccggaaacaagtgcagatgtcggggagggggtcatgggctctgacaaggaaacttgcagagtatccaaagcacaaggggacgaacctgttcaccagcggggcacgggtgggtgtccccgctcagccagcttccggaggatgggcagtgagagagtgagagaaggaaaggggcagccaggctgggggcttacggtgttgagagagagggcaggtgtgggaaagcagggtacggtttctacggcagcaaacagttccgagtgactggaaaaaagacagttgtcttcattcatcaggttgccataacaaaacactgcaggctggttggcttataagcaaaaggaaggttcaccttacggttctggaggctgggaagtccatgatccaggctctggcagatctggtgtttggtgagggcctgttccctggtcgatggtgccttctcactgtgtccttacatggtgaagaggcaaggcagctctctggggtccctttcctaaggacagcaatcccactcctgagggctccaccctcataacttaaccacacctcaaaggccccatcgcctgatacgtcatgttgagggttaggatttcagcgtatggattttggggcacacaaacattcagaccatagaaatgggagaagaagagtaagacagagagaaaagtaggcagaactactcattaagagtgaggtggaaggtggagagggcagggagaccttatgaagatcacacatctgtgaggaaggtttgcacactctcactgaagccccggtaggaggctcagcacccatttctctagatagcaccttccttcctctgaggacagattctgactggagccctggtagggggctcagcacccatccctctagatggcacctgccttactctgaggacagactctcactgaagccccggtaggaggctcagcacccatttctctagatagcaccttccttcctctgaggacagactctgattggagccctggcagggggctcagcacccatttctctagatagcaccttccttcctctgaggacagactctcactgaagccccggtaggaggctcagcacccatttctctagatagcaccttccttcctctgaggacagactctgattggagccctggcagggggctcagcacccatccctctagatggcacctgccttactctgaggacagactctcactgaagccccggtaggaggctcagcaaccatttctctagatagcaccttccttcctctgaggacagactctgattggagccctggcagggggctcagcacccatttctctagatagcaccttccttcctctgaggacagactctcactgaagccccggtaggaggctcagcacccatttctctagatagcaccttccttcctcaggacagactctgactggaaccctggtagggggctcagcacccatccctctagatggcacctgccttactctgaggacagactctgacgggagccctggtagggggctcagcacccatccctctagatggcacctgccttactctgaggacagactctgactggagccctggtagggggctcagcacccatctttctagatggcagctgccttactctgagggcaggagcttgctttgtatgttccagtcccttctgagaaccagaagcctgagcatctattagaaatgcaggattgtggacccaatcttagacctagagagctagactttgcatttgaatgagagctacaggtgattcaaaggtgactgcatgtgtgggtgcagtggctcttgcctgtaatcctaggttttgggaggccaagatggggggattgcttgaagccaggaattcaagtcctacctgagcagcaaagtgagagcacctctctacagaaaaacaaaagcaaaaatgtgactgcatgttggagccacctggctttttttaaataaaaaagatccatatagctggacacagtgactcgtgcctgtaacctcagctactcgggaggctgaggcaagaggattgcttgagcccaggagtttgaggctgcagtgagctatgatcatggcactgcattccagcttgggtttcatctgaaaaaaaatttaaaaaattcatctagttccacctcagagcttctgattttatttccatggatgtggcctgggcttgcgattttctttttcttttttttttttttgagatgaagtcttgctctgttgcccaggctggagtgctgtggcatgatcttggctcactgcaacttccacctcccaggttcaagtgattctcctgcctcagcctccctaatagctgggatgacaggctcgtgccaccacacccaattaatttttgtatttttagtagagagagggtttcaccatgttggccaggctgatcttgaactcctgacctcaggtgatctgcccgcctcggcctcccaaagtgctgggattacaggtgtgagccaccatgcccagcctggcctgaggatttttgtgagcactcctgatgctgttgtgtagccaggagtgaggactgccctctgatcacccagcacagaacgttcagggccatgttgggacagtggactggtaagggcgagaggaggacactggggagcaggctgtagggtgccacagtccgccacagtcacaggctcatgctctccgtccgagaggaggcagaactgcttttaccaaggctcatccagccctcttcatttctctgggaacaatttttggtttcgtgacccatttcttaatccccggaaggcatttctctgaactggtgccaggcaccccggcccccagctccagttagttattattcgaggctgatttcttttctgtctgtcttccttctaattcctagattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggtgagtccccgtgcatgtgatctgtgtgagtgcaagttcatatgagagagagagagagaaacagactggagaaagatcttcaggaccaggttgctggtgtggtttctttcttttctttttctttttttctttttttgaggcagagttttgctcttattgcccaggctggagtgcaatgccgtgacctcggctcactgcaatctctgcctcctgggttcaagtgattctcctgcctcagcctcctgagtagctgggattacaggcacccaccaccatacccagctaattttttgtatttttagtagagatggggtttcatcatgttgtccaggctagtctcgaagtcctgacctcaggtgatccgcccgcctcggcctcccaaaatgctgggattacaggcatgagccaccgcacccggccggtggtgtggtttctgctgtaacaaagtgccacagactgaatggcttcaacaacaaaaattcattttcttgcagttctgaggccgaaagtctaagatcaaggtgtcagcagggttggtttcttctgaggccgccctccttggtttgtagatgcccccttctccctgtgtactgatgccatcttgccttggggggaagagtggcctagtctcctcttcttacagggacagcagtcctgatggattaggcacagtcaggcatgactacacaagacctaatgttaatctagtcactcctttaaagaccccatttcccaaaacagccacattatgaggtcttgaggattagggcttcaacatgtaaatttttgggggggcacagttcagcccagaacaagtgagaaggtagaatgggttggaggccagctggggaagttttgtgtggggaagaatgaatgaggaactaaaaaaaaagtcatttcttgaaacaaccatgatgctcgatatattccatttcctcatgggccatgttcatagatgagagttggagaatttgccatttacaacaatggcaacctcacctaaaaactatgaaaatactttttttttttttctttgagacaggacctctctctgtcacccaggctggagtgtgtgatcacagcttactgcagcctcgacctcccaggctcaagcaatcctcccacctcagctcccccgagtagctgggactacaggcatgcaccaccatgctggctaatttttgtattttttttgtagagatgtggtctcatcacgttgcccagcctggtcagaatgctttgtatgggttgaaatgtcacagggaatctcctgtgactttattaataatgtgcttgctcacacctgtagtcccagctactcgggaggctgaggcaggagaactgtgtgtacccgggaggcggagatggcagtgagccgagatcgtgccattgcactccagcctgggtgacagagcgagattccatttcaaaataataataataataataataataaaaaatgtgcttgctttctcccacaccatgaatcagattcacacgccccaccgcgctggtgcctgggccgcctcactagctctgtttcattccctgctctgtggacgcatgcaggcgtctgcatttttgaggagctgcacagcaagtgttaaagtccgccactctaggacattttgctacctccctctccggctcaacacactcaccttgtcaccagtcttatcaatttttcctcctacccgagctgttttccacttttcctcgtcctcaccccaccagcccaggtccagcaactgtcaccacttgcctggacacgcagtgacctcctagaccatcagctccccggggatggggtttgcgtttgtttgtcttgctgtcatctttgccacgcctggacagacatcttagtacccatcagtggtcaaaagcacgcactgaatgaatgttggctctttggtctccctgtgtctgcctttttccctcacccgaccattctctgcacaaaagtaagatggtcctttagaaaattctgattcgattgtgctgttcggtggcttaaatgctttctgtctgctctgctctttattttttactttatgtatttatttttgtagatgtagggggcgcaagagcagctttgtttcatggatatgttactgcacagtggtagtgtctgggcttttactgtaactacccctttttttttgagatggactctcgccgtgttgcccaggctggagtacagtggagtgatctcggctcactgcaacatccacctcctgggttcaagcaattcttcgagtagttgggattacaggcatgggccaccacgcccagctgatttttgtatttttagtagggacaaggtttcaccgtattggctaggctggtctcaaactcctgacctcgtgatccacccgcctcggcctcccaaagtgctgggattacaggcgtgagccaccgtgcccggccttagtgtaactcttacccaaatagtgaacattgtacccaataaataattttccagccctcacctggctcccaccctcccacccttctgaggctctagtgtctatcattccatgctctatggatgttaccctttgtttagctcccacttgtaagtgagaacgtgtggtatttgactctgtttctgagttgtttcacttaaggtaatggcctccagttccatctgtgtggcttcaaagatgtgacttcattcttttctatggctgagcagtattccattatgtatatagatcacactcttcatctttgctcttttggtaaatttcaaatcgtagtgccatggagactggttgtttgggacatttaaccttatttctccaacttcatgcccaaattctctccccactgccattgcccctgtatgattctggagaggcagacacaaacagggaagggtaggggacagtgagatgcttcgcagagaagtagagatccatttgtccatttgcttgaagttggtgtaaggatagggacagaaaggaaggtggtctaacaggctccaggacctatgaactattgccctggaaagaagctagaatgttgttcttgcccccatgacagggataaagtatagtttttgttgctggtggtggtggtgacggtggtagtgatgtgcgtgtgtgtttgtgtgcatgtgtgtgacggggaaactaatcaagaatcagagctgcagcgggatgtggaatcccagcactttgagaggctgaggtgggcagatcacctgagcccaggagttcaaaaccagcctgagcaagctggtgagaccttgtctttagaagataataataataataaaaataaaatataaataaaataaaatggctaagcatggtagcacatgcctgtatttgcagccacttgggaggctgaggcaggtggatcccttgagcccaggagttcgaggtggcagtgagctatgatctcgccagtgcactccagcctgtgcaacaaagtgagaccctgtctttaaaaaaagaaaagaaaagaaaagaaaaagaatcagagctgcttttcctttcaatatagatggagggctgggatgaaatcgtcaaacacaatggggtgacaatgttggtgaagacaagaaatagctgagtgcagcactggcgagggcagtgccgactgcgggcacgtcaatggttaggaacacgagggaaggaggtgggtgcacaggcccacaccgcccgcttcacttacaaaccaggagcgcaccattcacatgtctggggggtgatctcaatgcacccggcactgtttacggccgggagttcactgacaggtgatccctctgctgtgactggggagactgggggctgacctcccaatgtgccttgtgctgtattccggagctctctggtcaggtgtattctgatgctttctctattccagagctctctggtcaggtgtgtcctgatgctttctctattccagagctctctggtcaggtgtgtcctgatgctttctctattccggagctctctggtcaggtgtgtcctgatgctttctctattccggagctctctggtcaggtgtgtcctgatgcattctctattccagagctctctggtcaggtgtgtcctgatgctttctctattccagagctctcttgtcaggtgtgtcctgatgctttctctattctggagctctctggtcagatgtgttctgatgctttctctattccggagctctctggtcaggtgtgttctgatgctttctgtattccggagctctctgatcagatgtgttctgatgctttctctattccggagcttcctgatcaggtgtgtcctgatgcttcctctgttctggagctctctggtcaggtgtgttctgatgctttctgcctctgttcttggcatgaaggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggtgagtcgagcccgttcccctaaggaagttctgccagcgaggcgtgctgcgcactctggcttcagtgaggcccggtgggctggggtttgagtgagaatggccttggcggctcgggtgactgcgtgtgcttgtcttggggtgtctagcattgcggtcagtgagtgtttgtcgtggggaaagccacacactcaaccctgacaaggagcataaataataacaaaaagaagatgtctgcctttagggtgcctactgcctagctgacaaaaagaaaaaatcagtgtaatgcattgctttagaggagttttagaacttcatgacaagccctgggtcctcatattgtataagaaaaatggtgctaaagcaatgcagaccctcgatgttgtatgcagcccttccctgagtgcttctgggagacttgcattttgaaggagagaaaaaaccacttaaaaatggaagggccatttctcacccattaggatggctattatcaaacaaaagcaagaaaactagaaaacaacaagtattggtgtgggtgcggagaaatggaagccattgtgtgttgctggtgggaatgtaaatgatgcagccactatggaaaacagcacagcaattcctaaaaaataaatacagaattactactggagccagcatttccactgctgggtatatctgcaaaagagttcaaagcaggaactcagccaggagcggtggctcacacctgtaatcccagcattttgggaggctgaggcaggtggatcactggaggtcgggagttcgagactagcctggccatcatagtaaaaccttgtctctactaaaaatacaaaaattagccaggcatggtggtgcatgcctgtaatcccagttactcggggtggaggcaagagaatcttttgaacctgggaggcgggggttgcagtgagtcaagatcccgtcactgtactcccgcctgggtgacagaggaagcattaaaaaaaacagaaggcaggaactcaaacatgttatttgtacatctatgtccatagcaacactattagaaatagccaaaaggtggaaataaccaaacgtccttcaacggatgaagggtgttgaaaagtgctatatatgtacattcagctttaaaaagaattctagtacacactacaacatggatgagcctcggggacattatgctaagtaagataagctagactcgaaagggcaaatactctgattgtccttataggaggtgtctagaacagacagattcatagagacaggaagaaacgtggttgcaggcaatgggagggcggagagtgggaagacattgtttaatggttatggagtttcagttctgcaagatgaaaggagttctggagatggtggtggtgatggttgcacaacaatgtgaagtgcttaaggccattaattgtgcacttaaaaatggttaaaatggtaaattttattttacacgtattttaccacaataaagctgaatgtagggagctgggaagatgtagtattggtgggagcttggagaatgcttacttgttattccaccaaatggaaaaggaaatcagaagtgtacatagagcttgtgtccactccctttccatgtgtaaactggagtagaggcagtggcaggtacgggtgcttccttgtccatggtggagcgtgggtgagagacatgactgacattctgccatctctatggaaggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggtaagtcttcatgggtgtcttaccagaaaagtaacttcttttcagaggtgaattatttggaaaatgtggatgggggttactttggtcaggcagagctgaaggtagagtaaggaggtaaaaacatcagaaatgctttgaaggactggaggcatataatgtatactgctgctaataagataatttatgctaatttatgattatagaactagagtttcttagtgttggaagaaaccttagagatggtgcagcccaattgcctgttgagggaaaaacttgcttccaagacactcacatgaatagtcacccattttatgtgtgcatgcttgcaagcatgcaagttatcgcatagtgcatgcagcctctacactaggaaagagctctcaccctgcagcctctacactaggagaggactcttacactgcagcctctatatcctcaacagtgtcatggctcagcaccttcacctatgtagccatgctctcccaggtacacaggctggtttgttagtgtctgtctttgaaatgtggtgcccagacctgatgatgcatggtggatgttttacctcctgttgcagagaactatggtttcactggtggaatctctgatttccttgttttttttggcagttacataagccttttgattcatgttggaattctgcagtaaacagtagtagggaatacccactaataatcatgcactttccctgtaccactttcctgtatcactttccctgtatcacctgctctttcatgttcttacatggattttctcagttggcttcagaaagatgctatagggtagggtatctgtgtctccaatttaaagaggagaaaataaaattcagggagcctcataactttgtccaaggacactcagagagaaggtagtggaactcagtgtagacaaacgtctgtgacctcccaagccactgccttaattcccagtgcattgccttaggagatgttgctaatgggctctctacagggcttcttcatctggctctcagcttaagggtgggacctccaatttagctttgctaaacctcatcttacctgtcttttgacccattgactaggcagttttgaatcttcattctgacaataatatctacaaatacaataagcaaattggataggtcttcattcattcacattattaatatatataccagccagatcagcattcaagacaaaatttggcaggaaaggtcttcccagaggagtaatccactaatccataattaacaaccaacctctgttcacaaagctctgatacttccttggacctctcccatcagccaatcctcagatgtgaccatatctctttctatgaagtcttcctgcagtgccttcatttcctcgccttagggtattacccttccttagcaaatcgccttagaatattactcttccttggcaatttgacttctgcttctctaagtttgttcaacagacatgattttgtcccttcattttgcaggccctctgggggctctgtggtgggcactgacagcagcagtgagtaaaacagaccaaaatcctgttttcacaaccgttcttttagggaagtgataacagataatagacatgctaaacaatataccctgtgtataagagctataagaaaaactggagtgacggcgaggggtgcttctctgctcacgtttggcgagtgcctctccgagcgggtgacattggagcaggtgctcagtgaagcgtgggatgggctgtgaaggagccggaagagccctccaggcaaagcagatggaaccgaagaggcccaggcgtaggcactcaggtgactctcagtgatcctggttccaaattccacatgacaaacaagccaattacaaatgtgcctgatgcatctgtaatatgctctatgtagttactattgtcaatacctaacctggtagagctgctgtaataaagtatcacaaacagagcggcctaaacaacaagagtttattgcctctcagttcaggaggccaaagtctgagactgaggcatcagcaggctgggtccttataggtcgtacaggtccttcccagtccttactttagttgacatctgctgcctttaattcatttcactcttcctctttggaagccttcttcgctcttatttttaggtcactacttagtctttcctgctaatgttatagcttccccctccctggatcctgcattggcttctcttcctgtggtctggggatttcacgactggtttagttgtgtttttttttttttttttttttttgagttggagtcttactctgtcacccaggctaaaatgcagtggcgcaatcttggctcactgcaacctctgcctcccgggttccaacaattcttctgcctcagccttctgagtagcagggactacaggtgtgcactgccatgcctggctaatttttgtatttttagtagatacggggtttcaccatgtttgccaggctggtctcgaatttctgacctcaggtgatctgcctgccttggcctcccaaagtgttgggattacaggcgtgagccacacacccgcctggtttaccttttttgttgttgttgttactaaatatttctaaactccctgagaattctcatatacctctcaatttcaacaacagtaggtctcaaacccagatatcattcacaaactttttattagaaaagtaacacatgcttggtgtaaacatatcatagtacagacaaagtggaaagtgaaagctccttctcagccctcttctgagagagccatttacaatagtctgatgaacaaccatccagactttttttaatacaatagattgttatataatccattcatctgtccatccatccatccatccatcatccatccctctattttccatccgtcaatccatccatccatccatccatccatccatccatccatccatcaatccatccaagggagtatcagagctttgtgaacatatattggttgctaattatggacaaatggatttcctgtatatgctgatctgggtggtatttatatcagtaatgtgaatgaagacctatgtgattttcatgttgcatttgtaatacattgttatgtagtattttaaatgcctagtctgttagagctactgtaataaagtatcacaaacagagcagcttaaacaacagaaatttattgtccctcagttctggaggccagaagtgtgagattaagatgctagcaggctgggtccttctgagggctgtggggaatctgttcatgcttctctttggcttctggtggaatgtcatcttgctgacacattttgccattccttgacctgtagatgcctctgccacaccattgcctttatctctacgtggtgtcttccctgtgtctgtgtgtgtctttctccatgtcccaagtttccctgtgaaagaacacaagtcatgttagatttgtaacgacctgatcttaactaattacattttcagtaaccgtatcaccaaataaggtcacaatctgaagtactggggcctaggacttccacatataaagtcttagaggaaacaattcaatccataacaccctatctattgaaactagaaccataatataaatatttttctgtgatatccttttttcatttatcatggataaatttccatgctgatttgtgtagatctatcgattggggcataatatcccatagtgtgctttaccataatttatttaatcaaatcatgcattttgggataactgttcttttccactattacaaacaactctgcaaaaagcacatttgtatatatagttttatgcatgtgtatttgctttgagataggtatctataagtggcattaatcagtcgagacttttatgcattttatgtgttaggtacagccatacatgtggctgtgtgttaggtatagcctatgggttaggcatagcctgtgtgttaggtatagcctatgggttaggtatagcctacgtgttaggtatagcctatgtgttaggtatagcctatgtgttaggtatagctacatttttttctctcgaactttgggtcctcatctctggctgcatattgtgtactgcctgtctcaatactgctcaagggtcacacatgctgagcatttgcaacattggtttaattatcataccccaccttccacccccgttctctgacctgctcctgccctcttcctcctgcctcattgaatagtggggctttccaggaggctgctgagtgagtaaacgggggacggtcctaaatgctctgctttcggttccctctaccactcatcactacaccagagggaactttttaagatgcaaatttgaaaatattgaccctttgttcaaaaatcttcaaaagttcttctctgcagtcagagtgacatcttagcttggcacacaaggctgtccctgatactgccctctctcctgccataccttgcactccattttgagtcatattggatgagttttgttttgctgttttagagatggggacttgctatgatgctcaggctgaatttgaattcctgggttcatctacagccataccacccagagtgcctgatctcatctgcactcctgggctcaagcaatcctcccgtctcagccgcccaagtagctgggactataggtggacatcactgtgcccagctatgaattagttttgaaacatcttggtggttcttttaatcttccttattcttgctactcttttccttttgcctggaaggttacccccactttcccactttctactatccatctaagaagctcctacttattttttaattactgtgatcctcattttgtccatttaaaagcagggataaaactaacattcccatcttgaggttgtgatcattcaatgagataaccctgtttaaagtcgtcatccatgtcaagcacacaataaatactcaaaaaatgtttgatcttcttcttcagattccttccttccttccttccttcttcctcctcctcctttctttctctctttttctttttctctttctttctttctttctctctctctctctttctttctttctctttctcccttcccttcccctcccccccccctttccctttcccctttcctttccttattttcctttcttttcttttctttttttaagacagggttttgctctgtggctcagactggagtgcagtggtaggatcatagctcactgtagccttgaactctcctgggctcaagcaatcttcctgcctcagccttctgactagctaggactacaggtgtgtactaccatgcctgggtaatgtttaaaatgtttaaaattttttatgtagagacagggtcttgctatgttgcccaggctggtctcaaactcctggcctcaagtggtcctcccaccttggcctcccaaagcactgggattcagttttcattatgagacagttttgtgacataggcaggtagttaataaataaaaatccccaaccaattaacttaatacagtacatggcaagatatccgtgcttggtaaatgtagagctcagttaggtggattggttgaaatcgagatgtcaatcacattcctctaatccaaggattggttggtgggacatggttgtttgattctttaaacattttattatgtgttcagtgtaaccgttgcttaatagttaccagcattttcacaatttggatatgacgttttcttaacaaaactgcaccgtctgttgcaatttctactactactgtgtagtgctccccaaacatctgttgaataatccattctactcctttttggaaatagtggtaaagcctgcataactttaattacataatttaaaaaatattttctcttttcctctttgaagaccatccccattctactagaatgtccctggcctttgccaggaatgctactaatcccttctgcatgttaattaacaacaaccctctgatagaattcttaccggggatgagagcttagtagaagccactcaaaacttgctttgtttttgtctgtctctcttgggcttcatttgcctattagtcttacttactgtgtcttttcagtttgactcttccttaattgggaagatagaaaccagtggatggataatcactttttcctgctggtcatctgttcatgctttactgtctgaatacactttttctgtatttctatttttcctgcctcgtagacaccaaagagcttaaaaaacatgccattgccaaggactctggcaggaaggaggcaatcacctgcgctattcctggcttcatggaatagaagcatctgtgctcctaggaattacgccaccagtgattttagagctcttatgctcttcctgggtgatagaatgggcttaatttgactcaagaatagtacatcaaaactcttaattccatcgagattgatgcatttttggggttttatgctctaaaaggccttttgctgtgcagaaggaagagagaggtggacagaaagggcagtggggagaagatgagactaaaaggtggaaagtccaggtctggatccctccccaccacacacctgctctgtggccttgagagaaacatgtaatttttataagccttagatttctaatatgtaaaatgtatgtaatagttttagctgcactccttccttgccaggttgatataataatcaaataagactggtctctgggtcaggcacagtggctcatgcctgtaatcccagcactttgggaggccaaggtgggtggattgtctgagcccaggagtttaagactgcagtgagctgtgcacttggcctgggtgactgagcaagagcctggctttttctatttatttatttatttttgagacagagtctcactttgttgcccaggttgaagtgcagtggcaatctcggctcactgcaacctctgcctcccaggttcaagcaattctcctgcctcagcctcccaagtagctgggattacaggtgcccaccaccaccctaactaatttttatatttttagtagagatgggctttcaccatgttggccaggctggttttgaactcctgacctcgtgatccaccctcccaaagtgctggacctcccaaagtgctgggattacaggcataagccactgcacctggccttatttatttatttatttaggcagggtctcactctgtcacccaggctagagtgtggcagcgcaatcatggctcactgcagcctcgacttcctggactcaagcgatcctcccacctctccaacgtggttgagggctgcagcgagaaagatgtgaaaggggcatttgggggcgcaggaaggcgggaggtggcaattgctaagggccccgccgtgttcctgtcacagccctagctacagcttagaaggtcacaatggacgttcacctgttgtctttgatgtttaaatcagcaccgacttcccatgaggaggatttttttttcctgtctctcttaaaaaaaaaaaaaaaggcccctgcagtgtgggtgtctgcagtgttacggggattattagttgaggataaccactgggatgtgtttctggaagaagtggtgctgaggagcactcctggccggaggaaagctgtgtgaggaggaggaggagtatggggtaactggctcagtttgccttggctctttctgtcggactcatacagcaaagcatctgctgagacatcatttgtgtctcctgtgacagccagagacagcaggtcttgctctccccagatcatatctgagatgctggctcctgctctctgagctgaaggctgcagctgctgagagaggacgaggcactgagtcaaagcagctgcacaatgttggggaacagctgggtactgaggactcttctccctgtccttctacaggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggagtaagtcctttggtttattacagcaatgagaacacatggtggccaagggtggagggacgtttaggcagagtggccacaagataatctgtatcttagaagtgaggtgtcttcttagtgtttgccttgttacaggatcatgggactgggaggagtccttccagatgatatagatgtaggaacttgagtccttaccatcacagttttctgtaccacaaaacaaggagggcagtatgacacttacattttatggaaatgtagggaaaggcacagaatttggtaaatcaatgaacaaaccactgttactaatttgatctgtgcaatgcagtggatttgaaaaagagagagaagaataattaccagcatacaaggctgcttgaagctagttagtcctgtgctcctgtgctttttttttttccacctgaaacaagacaggctaagatgctaaaatcttggggagctagggggatggttaaggggacattttctttaaatcaccccctttttgcagattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggtgagtgtgctctgcagagatgcccgtggtgggactctgagttctcaggaaacgttgactgttatcaaaatccaggatggctctcgcttcatttgcagccactcaagattaggttgggcctgggtcagttgtggtggatattttaaaatagagaatatggggttaaactacacattccacttcactgagatggttagaatcaccaaggctaagctggatttcacccacattccccaaagaaacacaagccctcatgcgatcatggccaaggaggattctgttctacacaaggaaccgaacttcgtagaactcgtctatgggtcatccaaattggaagggtctttacatagtggggatcttgttgttttgtttctgaggatttattttttttttccttgagacagagtcttgctctgtcgcccaggctggagtgcagtggcatgatctcggctcactgcaagctccgcctcccgggttcacgccattctcctgcctcggcctcccaagtagctgggaccacaggcgcctgccaccacacccggctaattttttgtatttttagtagagttggggtttcactgtgttagccaggatggtctccatctcctgacctcgtgatctgcctgcctcggcctccccaagtgctgggattacaggtgtgagcctccgtgcctggcggtttctgaggatttctaaatgggaaaagagtgctccaggaatagcaagatgggaaattacacgaaattcgacagtgtgtcaggatattgccagcccacctctgtgatactactgaggtagtcgtctcctctctagcaagagcttcctggcgagtcccacggttctgtcagccgtgatgattgcacttttccctgaagacgttttcactgcttggctttaaggccaacatgctctcctgggttttctcctatttcatcagatccttctactcagtctcctttctggcctcttcttcatcatcctgacccttatgtgttgaatcctcagagcttagtcgctgggctaatttttcttccctacactttctccctcatttaaattatctggtcttctggcttcatgatatgtgtgctgtggcctccagattatatgtccataaattctgtgtctccgtgcttggctgatctttcttcttgaaggtttaataaacctctcaaacgtcacatctagatccacacccttgctactctcttctacccgaaacctgattcttccccatttcagtcgatggtcacttcctccaagttgctcgggccaaaacatttctggttctgtcgtcactgtctctttttccttataatcatgtccaatgtgtcagcacatcctgtgagctctgccttcccagcttggccactccctgaccacttcaggccagcttcacggcttcttccttggctcaagccaccatcacctggtgtctggattgtcatggtggcattctgacaggtcaccctgcatctgttattctgattttagcagacaagggattcagctaaaacgtaactcgcatcatatcactcttgtcaacatcgtcttcttatcactttcataatcagagcaacaaactcatggtggcttccatgccccacataatccagcctctcctgcttccgaagattccgcttctcccaacccttgcttattctgccccagtacagtgatctccttgctgttttgcaagaacactgtcatctctggatttttgcacttgcttttcttctggcctagaactctctccccttcagtatccacctgcattattccctgtaccttcctaaattctctgcttaaaggtcttctctgcgaggctgtctttgactattctactgaaaatagaagcactttttcttctcttctcattctcctctgcagtgtagcacatcttatgtgctatgtcattttcttgtttatttattatctatctctgctcccaaaatataaattccattagtccagggacttctgtccatattaatgatctcaaggctgagaacttgttggaatgcaggtttgaataaatacaagtttgaatcagccttgcatccttgtgccaacataatggagcacatttgctgcaggacaagccaatattcccagagctaagagagatgggggaacaaacaaaggattggtcaggtcaaagctggggacactgctacatgtagcatcactgacctagccctgagggacattctggacccaggctgagggacaggctcaaaatagattgaagaatccatgaatgcaatcaacccgttttctcaggctgcgataacaagctgctttagactgtgtgactcaaacacagatttattatctcacaatactggaggctagaaagtccaaaatcaagtctctgatgaatttggttgctggcgagagtcctcttcctgatttgcagatggctaccttcttgttatgtgattgcatggagggaggggaggcaagggagagaagacaggggagaaagaacaaactctctgatatctctcctgtaagggcactcaccccatactgagggccccaccctcacaacttcatctaactctaactacttcccaagggcctcacctttaagtaccgtacattaggggttcgggcttcatcatgggaattgttaaggaagacaaacattcagtatataacaccaccaaattgagatggttataaatggtgcaggacaaatgggtggatttattagttaagttgcaagggaaactttattaaagaggaggaagaacttacatcgaagatgtgagaggcatcctgggcaaatgcaatgtgtagaattgatcaggttagtgactaaatgaacataaggtaaaaaaatttacaagacagttgaggaagcgtaaatgctgactggacatacagtgatactaagaaattattgttaaacgattttggtgtgatagtgacattgtaattatattttagtgtcacaaagtgaattattgatagatgcattaaatgcagtatttaaaataatccatgggtgggaggagtgggtgggcaatagaagaaacaagttgacatgacctgtttaattgtcgaacttgggtaatggacacagggggttcattatactattttctatacttttatgtatgcttaacattttcccaactagatagttaaaaatataacaggaaccacattgggcacttagagcttttttttttttttccccacatttttattgggagccgtgggaggggcctcctctgtcattggaggtgctcacagtttcttcagccactccaggctggggtcctgggggtctgaggctcccagtgacgaaggacacgacgagcaccggctccttggcccaggcattcttgaagaaggtgcggagtctcgcggccatcttggtctcggcggcggcgacggcggcgaggacgcggagcactctgggagttgtggtccctcttttttttttttttttttttttacattttagaagtgtgtggagtttagggaaatgaagagatgagacttttaaggttaacgactctgacagagttatctgaagagtgcaacccaggctttctatttgcttttacaggttgtctgaaatgtatttcaattatgagacaaaaagtgcgttagaaacacttcaaga

SEQ ID NO: 54 - DNA, 4187 bp, synthetic construct
  source 1..4187: mol_type = other DNA
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 55 - DNA, 3845 bp, synthetic construct
  source 1..3845: mol_type = other DNA
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 56 - DNA, 3545 bp, synthetic construct
  source 1..3545: mol_type = other DNA
gttcctgaggctggcatctggggaaacctttcttccatggctcaggacacactcctggatcgagccaacaggagaactttctgtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 57 - DNA, 4016 bp, synthetic construct
  source 1..4016: mol_type = other DNA
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 58 - DNA, 4016 bp, synthetic construct
  source 1..4016: mol_type = other DNA
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 59 - DNA, 4187 bp, synthetic construct; identical to SEQ ID NO: 54
  source 1..4187: mol_type = other DNA

SEQ ID NO: 60 - DNA, 5610 bp, synthetic construct
  source 1..5610: mol_type = other DNA
gttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactcca

SEQ ID NO: 61 - DNA, 5610 bp, synthetic construct; identical to SEQ ID NO: 60
  source 1..5610: mol_type = other DNA

SEQ ID NO: 62 - DNA, 5478 bp, synthetic construct
  source 1..5478: mol_type = other DNA
gttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactcca

SEQ ID NO: 63 - DNA, 2650 bp, synthetic construct
  source 1..2650: mol_type = other DNA
gcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactcca

SEQ ID NO: 64 - DNA, 5388 bp, synthetic construct
  source 1..5388: mol_type = other DNA
gttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactcca

SEQ ID NO: 65 - DNA, 5098 bp, synthetic construct
  source 1..5098: mol_type = other DNA
gttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcaggcaagagccacgaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggaaggaacaccagccagccgtggaacctcaggtgcaacagagacgccaggagatactagtgcccagcagcctgcggcagtaccaatgaagccagagagggcttggtggatgacaaggaggcctgagtagaccgcaggtgggtctgagaaatgggcttaggtgaggcaggtctttgaaggatttgttcttaatcatatgcgagatgctcaaaaggctggatgcctgcttttgtgggtgaagagcaagaagagaaaacaggttgtacacatacagatgcagatggagagacagagaaaaaaaaggaagaaggcagagaaatgcaccaattcttgagctgtattatctctggaccttgggattgtgggaggctttattttactactgattttgcctacactgttttctcaatttctagttttctacaaagatgatgtgttagctttttcacgcattaagattaaaatttaaaacagacca